(19) United States
(12) Patent Application Publication
(10) Pub. No.: US 2026/0224629 A1
(43) Pub. Date: Aug. 6, 2026

(54) ARMORED IMMUNORESPONSIVE CELLS TARGETING DLL3 AND USES THEREOF

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US)

(72) Inventor: Renier J. BRENTJENS, Short Hills, NJ (US)

(73) Assignees: Memorial Sloan-Kettering Cancer Center, New York, NY (US); Sloan-Kettering Institute for Cancer Research, New Yorik, NY (US); Memorial Hospital for Cancer and Allied Diseases, New York, NY (US)

(21) Appl. No.: 19/651,189

(22) Filed: Apr. 17, 2026

Related U.S. Application Data
(63) Continuation of application No. PCT/US2024/052044, filed on Oct. 18, 2024.
(60) Provisional application No. 63/591,196, filed on Oct. 18, 2023.

(51) Int. Cl. A61K 35/17; A61K 40/11; A61K 40/31; A61K 40/42; A61P 35/00; C07K 14/54; C07K 14/705; C07K 16/22

(52) U.S. Cl. CPC A61K 35/17; A61K 40/11; A61K 40/31; A61K 40/4225; A61P 35/00; C07K 14/54; C07K 14/7051; C07K 16/22; A61K 2239/13; A61K 2239/21; A61K 2239/22; C07K 2317/565

(57) ABSTRACT

The presently disclosed subject matter provides for antigen-recognizing receptors that specifically target DLL3 and cells comprising such DLL3-targeted antigen-recognizing receptors. The presently disclosed subject matter further provides uses of the DLL3-targeted antigen-recognizing receptors for treatment.

### CROSS-REFERENCE TO RELATED APPLICATIONS

This application is a Continuation of International Patent Application No. PCT/US2024/052044, filed Oct. 18, 2024, which claims priority to U.S. Provisional Application Ser. No. 63/591,196, filed Oct. 18, 2023 to which priority is claimed and the contents of which are incorporated herein in their entireties.

### SEQUENCE LISTING

The present specification makes reference to a Sequence Listing (submitted electronically as a xml file named “072734_1700_ST26.xml” on Oct. 18, 2024). The 072734_1700_ST26.xml file was generated on Oct. 16, 2024 and is 196,747 bytes in size. The entire contents of the Sequence Listing are hereby incorporated by reference.

### INTRODUCTION

The presently disclosed subject matter provides methods and compositions for immunotherapies. It relates to immunoresponsive cells bearing antigen-recognizing receptors (e.g., chimeric antigen receptors (CARs)) that specifically target DLL3, that are engineered to express a cytokine (e.g., IL-18), and methods of using such cells for treatments.

### BACKGROUND OF THE INVENTION

Cell-based immunotherapy is a therapy with curative potential for the treatment of cancer. T cells and other immune cells may be modified to target tumor antigens through the introduction of genetic material coding for artificial or synthetic receptors for antigen, termed chimeric antigen receptors (CARs), specific to selected antigens. Targeted T cell therapy using CARs has shown recent clinical success in treating hematologic malignancies and solid tumors.

Small cell lung cancer (SCLC) accounts for about 15% of lung cancers, and two-thirds of cases are metastatic at the time of diagnosis. See Rudin et al., Nat Rev Dis Primers 7(1):3 (2021). Despite initial sensitivity to chemotherapy, metastatic SCLC almost always recurs, leading to a median overall survival of approximately 13 months. See Horn et al., N Engl J Med 379(23):2220-2229 (2018). DLL3 is selectively expressed in high grade pulmonary neuroendocrine tumors of the lung (LU-NETs) and other neuroendocrine cancers. Lu-NETs embrace a heterogeneous family of neoplasms classified into four histological variants, namely typical carcinoid (TC), atypical carcinoid (AC), large cell neuroendocrine carcinoma (LCNEC) and small cell lung carcinoma (SCLC). Increased expression of DLL3 was observed in SCLC and LCNEC patient-derived xenograft tumors and was also confirmed in primary tumors. See Saunders et al., Sci Translationa lMedicine (302): 302ra136 (2015). Both SCLC and pulmonary LCNEC are high-grade and poor-prognosis tumors, with higher incidence in smokers. Pulmonary LCNEC exhibits biologically aggressive behavior, similarly to SCLC. Stage by stage, survival curves of pulmonary LCNEC and SCLC overlap, and in addition, survival is lower than other NSCLCs. Prognosis is poor even in patients with potentially resectable stage I lung cancer with 5-year survival rates ranging from 27% to 67%. See Iyoda A. et al., J Thorac Cardiovasc Surg. 138:446-453 (2009).

Given the significant role for DLL3 in various diseases or disorders, immunotherapies (e.g., CARs) targeting DLL3, are desired.

### SUMMARY OF THE INVENTION

The presently disclosed subject matter provides cells comprising antigen-recognizing receptors that specifically target DLL3 and that secrete cytokines (e.g., IL-18). Furthermore, the presently disclosed subject matter provides uses of the presently disclosed cells for treatment.

In certain non-limiting embodiments, the presently disclosed subject matter provides cell comprising: a) an antigen-recognizing receptor comprises an extracellular antigen-binding domain, a transmembrane domain, and an intracellular signaling domain; and b) an exogenous IL-18 polypeptide; wherein the extracellular antigen-binding domain specifically binds to DLL3, wherein the extracellular antigen-binding domain comprises:
i) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
ii) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
iii) a heavy chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and a light chain variable region comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26;
iv) a heavy chain variable region comprising a CDR1 comprising the amino acid sequence set forth in SEQ ID NO: 11, a CDR2 comprising the amino acid sequence set forth in SEQ ID NO: 31, and a CDR3 comprising the amino acid sequence set forth in SEQ ID NO: 32; and a light chain variable region comprising a CDR1 comprising the amino acid sequence set forth in SEQ ID NO: 25, a CDR2 comprising the amino acid sequence set forth in SEQ ID NO: 33, and a CDR3 comprising the amino acid sequence set forth in SEQ ID NO: 34;
v) a heavy chain variable region comprising a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41, and a light chain variable region comprising a CDR1 comprising the amino acid sequence set forth in SEQ ID NO: 42, a CDR2 comprising the amino acid sequence set forth in SEQ ID NO: 43, and a CDR3 comprising the amino acid sequence set forth in SEQ ID NO: 44;
vi) a heavy chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51; and a light chain variable region comprising a CDR1 comprising the amino acid sequence set forth in SEQ ID NO: 52, a CDR2 comprising the amino acid sequence set forth in SEQ ID NO: 53, and a CDR3 comprising the amino acid sequence set forth in SEQ ID NO: 54;
vii) a heavy chain variable region comprising a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61; and a light chain variable region comprising a CDR1 comprising the amino acid sequence set forth in SEQ ID NO: 62, a CDR2 comprising the amino acid sequence set forth in SEQ ID NO: 63, and a CDR3 comprising the amino acid sequence set forth in SEQ ID NO: 64; or
viii) a heavy chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and a light chain variable region comprising a CDR1 comprising the amino acid sequence set forth in SEQ ID NO: 62, a CDR2 comprising the amino acid sequence set forth in SEQ ID NO: 72, and a CDR3 comprising the amino acid sequence set forth in SEQ ID NO: 73.

In certain embodiments, the extracellular antigen-binding domain comprises:
a) a heavy chain variable region comprising a CDR1 comprising the amino acid sequence set forth in SEQ ID NO: 1, a CDR2 comprising the amino acid sequence set forth in SEQ ID NO: 2 and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
b) a heavy chain variable region comprising a CDR1 comprising the amino acid sequence set forth in SEQ ID NO: 69, a CDR2 comprising an amino acid sequence set forth in SEQ ID NO: 70, and a CDR3 comprising the amino acid sequence set forth in SEQ ID NO: 71; and a light chain variable region comprising a CDR1 comprising the amino acid sequence set forth in SEQ ID NO: 62, a CDR2 comprising the amino acid sequence set forth in SEQ ID NO: 72, and a CDR3 comprising the amino acid sequence set forth in SEQ ID NO: 73.

In certain embodiments, the extracellular antigen-binding domain is a single-chain variable fragment (scFv). In certain embodiments, the extracellular antigen-binding domain is a human scFv. In certain embodiments, the extracellular antigen-binding domain is a Fab, which is optionally crosslinked. In certain embodiments, the extracellular antigen-binding domain is a F(ab)2. In certain embodiments, one or more of the scFv, Fab and F(ab)2 are comprised in a fusion protein with a heterologous sequence to form the extracellular antigen-binding domain.

In certain embodiments, the extracellular antigen-binding domain comprises a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical to the amino acid sequence set forth in SEQ ID NO: 7, SEQ ID NO: 17, SEQ ID NO: 27, SEQ ID NO: 35, SEQ ID NO: 45, SEQ ID NO: 55, SEQ ID NO: 65, or SEQ ID NO: 74. In certain embodiments, the extracellular antigen-binding domain comprises a heavy chain variable region comprising the amino acid sequence set forth in SEQ ID NO: 7, SEQ ID NO: 17, SEQ ID NO: 27, SEQ ID NO: 35, SEQ ID NO: 45, SEQ ID NO: 55, SEQ ID NO: 65, or SEQ ID NO: 74. In certain embodiments, the extracellular antigen-binding domain comprises a heavy chain variable region comprising the amino acid sequence set forth in SEQ ID NO: 7 or SEQ ID NO: 74.

In certain embodiments, the extracellular antigen-binding domain comprises a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% identical to the amino acid sequence set forth in SEQ ID NO: 8, SEQ ID NO: 18, SEQ ID NO: 28, SEQ ID NO: 36, SEQ ID NO: 46, SEQ ID NO: 56, SEQ ID NO: 66, or SEQ ID NO: 75. In certain embodiments, the extracellular antigen-binding domain comprises a light chain variable region comprising the amino acid sequence set forth in SEQ ID NO: 8, SEQ ID NO: 18, SEQ ID NO: 28, SEQ ID NO: 36, SEQ ID NO: 46, SEQ ID NO: 56, SEQ ID NO: 66, or SEQ ID NO: 75. In certain embodiments, the extracellular antigen-binding domain comprises a light chain variable region comprising the amino acid sequence set forth in SEQ ID NO: 8 or SEQ ID NO: 75.

In certain embodiments, the extracellular antigen-binding domain comprises: a) a heavy chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence selected set forth in SEQ ID NO: 7, SEQ ID NO: 17, SEQ ID NO: 27, SEQ ID NO: 35, SEQ ID NO: 45, SEQ ID NO: 55, SEQ ID NO: 65, or SEQ ID NO: 74; and b) a light chain variable region comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 8, SEQ ID NO: 18, SEQ ID NO: 28, SEQ ID NO: 36, SEQ ID NO: 46, SEQ ID NO: 56, SEQ ID NO: 66, or SEQ ID NO: 75.

In certain embodiments, the extracellular antigen-binding domain comprises: a) a heavy chain variable region comprising the amino acid sequence set forth in SEQ ID NO: 7, SEQ ID NO: 17, SEQ ID NO: 27, SEQ ID NO: 35, SEQ ID NO: 45, SEQ ID NO: 55, SEQ ID NO: 65, or SEQ ID NO: 74; and b) a light chain variable region comprising the amino acid sequence set forth in SEQ ID NO: 8, SEQ ID NO: 18, SEQ ID NO: 28, SEQ ID NO: 36, SEQ ID NO: 46, SEQ ID NO: 56, SEQ ID NO: 66, or SEQ ID NO: 75.

In certain embodiments, the extracellular antigen-binding domain comprises: a) a heavy chain variable region comprising the amino acid sequence set forth in SEQ ID NO: 7 or SEQ ID NO: 74; and b) a light chain variable region comprising the amino acid sequence set forth in SEQ ID NO: 8 or SEQ ID NO: 75.

In certain embodiments, the extracellular antigen-binding domain comprises:
a) a heavy chain variable region comprising the amino acid sequence set forth in SEQ ID NO: 7, and a light chain variable region comprising the amino acid sequence set forth in SEQ ID NO: 8;
b) a heavy chain variable region comprising the amino acid sequence set forth in SEQ ID NO: 17, and a light chain variable region comprising the amino acid sequence set forth in SEQ ID NO: 18;
c) a heavy chain variable region comprising the amino acid sequence set forth in SEQ ID NO: 27, and a light chain variable region comprising the amino acid sequence set forth in SEQ ID NO: 28;
d) a heavy chain variable region comprising the amino acid sequence set forth in SEQ ID NO: 35, and a light chain variable region comprising the amino acid sequence set forth in SEQ ID NO: 36;
e) a heavy chain variable region comprising the amino acid sequence set forth in SEQ ID NO: 45, and a light chain variable region comprising the amino acid sequence set forth in SEQ ID NO: 46;
f) a heavy chain variable region comprising the amino acid sequence set forth in SEQ ID NO: 55, and a light chain variable region comprising the amino acid sequence set forth in SEQ ID NO: 56;
g) a heavy chain variable region comprising the amino acid sequence set forth in SEQ ID NO: 65, and a light chain variable region comprising the amino acid sequence set forth in SEQ ID NO: 66; or
h) a heavy chain variable region comprising the amino acid sequence set forth in SEQ ID NO: 74, and a light chain variable region comprising the amino acid sequence set forth in SEQ ID NO: 75.

In certain embodiments, the extracellular antigen-binding domain comprises: a) a heavy chain variable region comprising the amino acid sequence set forth in SEQ ID NO: 7, and a light chain variable region comprising the amino acid sequence set forth in SEQ ID NO: 8; or b) a heavy chain variable region comprising the amino acid sequence set forth in SEQ ID NO: 74, and a light chain variable region comprising the amino acid sequence set forth in SEQ ID NO: 75.

In certain embodiments, the extracellular antigen-binding domain comprises a linker between a heavy chain variable region and a light chain variable region of the extracellular antigen-binding domain. In certain embodiments, the linker comprises or consists of the amino acid sequence set forth in SEQ ID NO: 78, SEQ ID NO: 79, SEQ ID NO: 80, SEQ ID NO: 81, SEQ ID NO: 82, or SEQ ID NO: 83. In certain embodiments, the extracellular antigen-binding domain comprises a signal peptide that is covalently joined to the 5′ terminus of the extracellular antigen-binding domain.

In certain embodiments, the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

In certain embodiments, the intracellular signaling domain comprises a CD3ζ polypeptide. In certain embodiments, the CD3ζ polypeptide comprises or consists of the amino acid sequence set forth in SEQ ID NO: 90.

In certain embodiments, the intracellular signaling domain further comprises at least one co-stimulatory signaling region. In certain embodiments, the at least one co-stimulatory signaling region comprises a CD28 polypeptide, a 4-1BB polypeptide, an OX40 polypeptide, an ICOS polypeptide, a DAP-10 polypeptide, or a combination thereof. In certain embodiments, the at least one co-stimulatory signaling region comprises a CD28 polypeptide.

In certain embodiments, the CD28 polypeptide comprises or consists of amino acids 180 to 220 of SEQ ID NO: 87. In certain embodiments, the CD28 polypeptide comprises a mutated YMNM motif. In certain embodiments, the CD28 polypeptide comprises or consists of the amino acid sequence set forth in SEQ ID NO: 143, SEQ ID NO: 144, SEQ ID NO: 145, SEQ ID NO: 146, or SEQ ID NO: 147.

In certain embodiments, the antigen-recognizing receptor is a chimeric antigen receptor (CAR), or a T-cell like fusion protein. In certain embodiments, the antigen-recognizing receptor is a CAR. In certain embodiments, the antigen-recognizing receptor is recombinantly expressed. In certain embodiments, the cell is transduced with the antigen-recognizing receptor. In certain embodiments, the antigen-recognizing receptor is constitutively expressed on the surface of the cell. In certain embodiments, the exogenous IL-18 polypeptide is a human IL-18 polypeptide.

In certain embodiments, the cell is an immunoresponsive cell. In certain embodiments, the cell is a cell of the lymphoid lineage or a cell of the myeloid lineage. In certain embodiments, the cell is selected from the group consisting of a T cell, a Natural Killer (NK) cell, and a stem cell from which a lymphoid cell may be differentiated. In certain embodiments, the cell is a T cell. In certain embodiments, the T cell is a cytotoxic T lymphocyte (CTL) or a regulatory T cell. In certain embodiments, the stem cell is a pluripotent stem cell. In certain embodiments, the pluripotent stem cell is an embryoid stem cell or an induced pluripotent stem cell.

In certain non-limiting embodiments, the presently disclosed subject matter provides a nucleic acid encoding a) an antigen-recognizing receptor comprises an extracellular antigen-binding domain, a transmembrane domain, and an intracellular signaling domain; and b) an exogenous IL-18 polypeptide; wherein the extracellular antigen-binding domain specifically binds to DLL3, wherein the extracellular antigen-binding domain comprises:
i) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
ii) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
iii) a heavy chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and a light chain variable region comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26;
iv) a heavy chain variable region comprising a CDR1 comprising the amino acid sequence set forth in SEQ ID NO: 11, a CDR2 comprising the amino acid sequence set forth in SEQ ID NO: 31, and a CDR3 comprising the amino acid sequence set forth in SEQ ID NO: 32; and a light chain variable region comprising a CDR1 comprising the amino acid sequence set forth in SEQ ID NO: 25, a CDR2 comprising the amino acid sequence set forth in SEQ ID NO: 33, and a CDR3 comprising the amino acid sequence set forth in SEQ ID NO: 34;
v) a heavy chain variable region comprising a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41, and a light chain variable region comprising a CDR1 comprising the amino acid sequence set forth in SEQ ID NO: 42, a CDR2 comprising the amino acid sequence set forth in SEQ ID NO: 43, and a CDR3 comprising the amino acid sequence set forth in SEQ ID NO: 44;
vi) a heavy chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51; and a light chain variable region comprising a CDR1 comprising the amino acid sequence set forth in SEQ ID NO: 52, a CDR2 comprising the amino acid sequence set forth in SEQ ID NO: 53, and a CDR3 comprising the amino acid sequence set forth in SEQ ID NO: 54;
vii) a heavy chain variable region comprising a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61; and a light chain variable region comprising a CDR1 comprising the amino acid sequence set forth in SEQ ID NO: 62, a CDR2 comprising the amino acid sequence set forth in SEQ ID NO: 63, and a CDR3 comprising the amino acid sequence set forth in SEQ ID NO: 64; or
viii) a heavy chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and a light chain variable region comprising a CDR1 comprising the amino acid sequence set forth in SEQ ID NO: 62, a CDR2 comprising the amino acid sequence set forth in SEQ ID NO: 72, and a CDR3 comprising the amino acid sequence set forth in SEQ ID NO: 73.

In certain non-limiting embodiments, the presently disclosed subject matter provides a vector comprising the nucleic acids disclosed herein. In certain embodiments, the vector is a γ-retroviral vector. In certain non-limiting embodiments, the presently disclosed subject matter provides a vector or a host cell comprising the nucleic acids disclosed herein. In certain embodiments, the host cell is a T cell.

In certain non-limiting embodiments, the presently disclosed subject matter provides a composition comprising the cells disclosed herein. In certain embodiments, the composition is a pharmaceutical composition further comprising a pharmaceutically acceptable carrier.

In certain non-limiting embodiments, the presently disclosed subject matter provides a lipid nanoparticle comprising the nucleic acid disclosed herein. In certain non-limiting embodiments, the presently disclosed subject matter provides a composition comprising the lipid nanoparticle disclosed herein. In certain embodiments, the composition is a pharmaceutical composition further comprising a pharmaceutically acceptable carrier.

In certain non-limiting embodiments, the presently disclosed subject matter provides a method of treating or ameliorating a disease or disorder in a subject, comprising administering to the subject the presently disclosed cell or composition. In certain embodiments, the disease or disorder is a tumor. In certain embodiments, the tumor is cancer. In certain embodiments, the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. In certain embodiments, the neuroendocrine tumors of the lung are selected from the group consisting of pulmonary neuroendocrine cancer, large cell neuroendocrine carcinoma, and small-cell lung cancer. In certain embodiments, the tumor is small-cell lung cancer. In certain embodiments, the subject is a human.

In certain non-limiting embodiments, the presently disclosed subject matter provides a kit for treating or ameliorating a disease or disorder in a subject, comprising the cell, the nucleic acid, the lipid nanoparticle, or the composition disclosed herein. In certain embodiments, the kit further comprises written instructions for using the cell or composition for treating or ameliorating a disease or disorder in a subject.

In certain non-limiting embodiments, the presently disclosed subject matter provides a method for producing a cell disclosed herein, comprising introducing into the cell a nucleic acid that encodes the antigen-recognizing receptor and an exogenous IL-18 polypeptide.

In certain non-limiting embodiments, the presently disclosed subject matter provides a the cell or the composition disclosed herein for use in treating or ameliorating a disease or disorder in a subject. In certain embodiments, the disease or disorder is a tumor. In certain embodiments, the tumor is cancer. In certain embodiments, the disease or disorder is selected from the group consisting of neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. In certain embodiments, the neuroendocrine tumors of the lung are selected from the group consisting of pulmonary neuroendocrine cancer, large cell neuroendocrine carcinoma, and small-cell lung cancer. In certain embodiments, the tumor is small-cell lung cancer. In certain embodiments, the subject is a human.

### BRIEF DESCRIPTION OF THE FIGURES

The following Detailed Description, given by way of example, but not intended to limit the invention to specific embodiments described, may be understood in conjunction with the accompanying drawings.

FIGS. 1A-1F illustrate the development of scFv for effective CAR T cells against DLL3 on small cell lung cancer. FIG. 1A is a schematic of the extracellular DLL3 domains with binding location of anti-DLL3 SC16 antibody clones (top), and schematic of CAR design for initial selection of single-chain variable fragments (bottom). LTR, long terminal repeats; CD8SP, CD8 signal peptide; V_{H}, heavy chain; V_{L}, light chain; TM, transmembrane domain; h, humanized. FIG. 1B illustrates luciferase killing assay with CAR T cells cocultured with DLL3-expressing H82-SCLC or DLL3—Set2 cells. The Set2 cells overexpress MUC16 as positive control for the MUC16-targeting 4H11 CAR (n=3). FIGS. 1C and 1D show DLL3-specific activation of the SC16.8, SC16.125, and SC16.126 CARs in cocultures with DLL3+293 cells, (FIG. 1C) as measured by IFN-γ production (see also FIG. 6 for IL-2, GM-CSF, and TNF-α levels) and (FIG. 1D) 7-day proliferation (n=3). 3T3-MUC16 cells were used as positive control for the 4H11 CAR. FIGS. 1E and F show orthotopic or metastatic H82-SCLC tumor growth following administration of the indicated CAR T cells (n=4-5).

FIGS. 2A-2K illustrate the effects of CAR T cell-secreted IL-12 and IL-18 on survival in a murine SCLC model. FIG. 2A shows luciferase killing assay with murine CAR T cells and mSCLC cells that endogenously express murine DLL3 (n=3). The CAR consists of the SC16.8 single-chain variable fragment (scFv) and murine CD28 and CD3ζ domains. FIG. 2B measures the IFN-γ levels in supernatant of 24-hour coculture of murine CAR T cells with or without mSCLC cells. **P<0.01, ****P<0.0001 (2-way ANOVA, n=2). FIG. 2C depicts the survival of mice with metastatic mSCLC that were treated with the indicated doses of SC16.8m28mz CAR T cells (day 7) with or without 50 mg/kg cyclophosphamide pretreatment (day 6). **P<0.01 (log-rank test, n=4-6). FIG. 2D is a schematic of CAR design with SC16.8 scFv, murine CD28 and CD3ζ signaling domains, and the murine 1112 fusion transgene or murine 1118 transgene. IRES, internal ribosome entry site. FIG. 2E illustrates IFN-γ production by the respective CAR T cells in the absence and presence of mSCLC cells. *P<0.05, **P<0.01, ***P<0.001, ****P<0.0001 (2-way ANOVA and Student's t test, n=2-3). FIG. 2F depicts a luciferase killing assay with SC16.8 CAR T cells that express IL-12, IL-18, or no cytokine and 4H11 CAR T cells as negative control (n=3). FIGS. 2G and 2H depict serum IFN-γ and TNF-α levels (n=12-15) (2G) and circulating CAR T cells (n=5) (2H) in the blood of mice collected 3 days after treatment with the indicated CAR T cells. **P<0.01, ***P<0.001, ****P<0.0001 (1-way ANOVA). FIG. 2I shows tumor growth in mice that were treated with 2×10⁶ CAR T cells intravenously 7 days after systemic mSCLC administration (n=12-15). FIG. 2J illustrates the survival of mice in FIG. 2I. *P<0.05, ****P<0.0001 (log-rank test). FIG. 2K shows DLL3 surface expression on endstage tumors from mice treated with SC16.8m28mz_mIL12 (left) or SC16.8m28mz_mIL18 (right) CAR T cells.

FIGS. 3A-3I Illustrate the effects of IL-18 on the activation of genetically engineered and endogenous T cells. FIG. 3A depicts the experimental setup for FIGS. 3B-3E, FIGS. 4A-4H, and FIG. 7A-7C. Briefly, 2×10⁶ murine mCherry-expressing SC16.8m28mz or SC16.8m28mz_mIL18 CAR T cells were administered 7 days after systemic mSCLC injection. 3, 6, or 10 days later livers and spleens were harvested for flow cytometry analysis (day 3 and day 6, n=6 from 2 independent experiments; day 10, n=3). FIG. 3B shows the levels of CAR T cells detected in the liver over time. FIG. 3C depicts an example (day 3) and quantification of the CD8⁺ CAR T cell population. FIGS. 3D and 3E depict an example (day 3) and quantification of (FIG. 3D) intracellular IFN-γ and TNF-α in CAR⁺ T cells and (FIG. 3E) CAR− endogenous T cells in the liver. FIG. 3F shows CD4⁺ or CD8⁺ mCherry-endogenous T cells were sorted on day 3 and 6 after CAR T cell treatment and cocultured with mSCLC, and IFN-γ release was measured with an ELISpot assay (n=3). For FIGS. 3B-3F, *P<0.05, **P<0.01, ***P<0.001, ****P<0.0001 (2-way ANOVA). FIG. 3G shows systemic mSCLC growth curve (left) and survival curve (right). Tumor-bearing mice were treated with low-dose 50 mg/kg cyclophosphamide (day 6) followed by low-dose 0.5×10⁶ CAR T cells (day 7). **P<0.01(log-rank test, n=5). FIG. 3H shows tumor growth of wild-type (n=3-4) or DLL3KO mSCLC (n=6, see also FIG. 7D) in long-term surviving mice after cyclophosphamide plus SC16.8m28mz_mIL18 treatment with no evidence of tumor on day 42 (see FIG. 3G) compared with age-matched naive control mice. *P<0.05, **P<0.01, ***P<0.001, ****P<0.0001 (Student's t test). FIG. 3I shows survival of mice in FIG. 3H. *P<0.05 (log-rank test, n=6).

FIGS. 4A-4I illustrate the effects of IL-18 on the recruitment and reprograming of myeloid cells in the tumor microenvironment. FIGS. 4A and 4B show the levels of CD11b⁺Gr1⁻ macrophages and MHC-II⁺CD11c⁺ dendritic cells detected (FIG. 4A) in the liver TME and (FIG. 4B) in the spleen 3, 6, and 10 days after CAR T cell treatment. *P<0.05, **P<0.01, ****P<0.0001 (2-way ANOVA). FIGS. 4C-H show representative flow plots and quantification on day 6 after CAR T cell administration. See FIG. 8 for quantification on day 3, 6, and 10. *P<0.05, **P<0.01, ***P<0.001, ****P<0.0001 (Student's t test). FIG. 4C shows the levels of CD206+MHC-IIlo M2-like macrophages. FIGS. 4D and 4E depict the expression of activation marker CD86 on (FIG. 4D) macrophages and (FIG. 4E) dendritic cells in liver TME and spleen. FIGS. 4F and 4G show expression of checkpoint molecule PD-L1 on (FIG. 4F) macrophages and (FIG. 4G) dendritic cells in liver TME and spleen. FIG. 4H measures the expression of PD-L1, MHC-II, and CD86 on F4/80⁺ macrophages in the liver TME. FIG. 4I depicts the survival of mice with metastatic mSCLC that received 2×10⁶ CAR T cells on day 7 and 250 μg anti-PD-L1 antibody on days 14, 17, and 20. **P<0.01,****P<0.0001 (log-rank test, n=5 for 4H11 and SC16.8, n=14 for SC16.8_mIL18).

FIGS. 5A-5L illustrate that human DLL3-targeting CAR T cells that secrete IL-18 are effective against SCLC and can be further potentiated by late-onset anti-PD-1 therapy. FIG. 5A is a schematic overview of the CAR design. EGFRt, truncated EGFR; scFv, single-chain variant fragment. FIG. 5B shows the levels of IL-18 and IFN-γ in cell culture supernatant of cocultures of the indicated CAR T cells and DLL3-expressing target cells. ***P<0.001, ****P<0.0001 (Student's t test, n=4). FIG. 5C depicts luciferase killing assay with H82 (DLL3+) and H69 (DLL3^{lo}) SCLC cells. FIG. 5D shows CAR T cell proliferation in cocultures with H82 or H69 SCLC cells (E/T ratio of 1:5, n=3, representative result from 3 independent experiments). FIG. 5E shows growth curve of metastatic H82-SCLC tumors in mice that received 1×10⁶ SC16.8 CAR T cells that did or did not secrete IL-18 (n=4-5). FIG. 5F-5J depicts the livers of mice were subjected to flow cytometry analysis 9 days after CAR T cell treatment of mice with metastatic H82-SCLC tumors. *P<0.05, **P<0.01, ***P<0.001, ****P<0.0001 (2-way ANOVA, n=3). FIG. 5F shows cell count of GFP⁺ H82 tumor cells (left) and PD-L1 expression on these GFP⁺ cells (right). FIG. 5G shows cell count of EGFRt⁺ CAR T cells in the liver (left) and CD8/CD4 ratio of the CAR T cells (right). FIG. 5H depicts quantification of CD62L⁺ central memory CAR T cells. FIG. 5I shows quantification of CD4⁺ and CD8⁺ CAR T cells that are quadruple negative (left) or quadruple positive (right) for the exhaustion markers PD-1, TIGIT, TIM3, and LAG3. FIG. 5J shows representative flow plots and mean fluorescence intensity (MFI) of T cell activation markers CD71 and HLA-DR on CAR T cells. FIG. 5K illustrates growth curve of metastatic H82-SCLC tumors in mice that received 0.3×10⁶ CAR T cells alone or in combination with twice weekly 250 μg anti-human anti-PD-1 antibody, starting on day 18 (n=4). FIG. 5L shows bioluminescence imaging of same mice as in FIG. 5K, on days 17, 21, and 26 after tumor cell injection.

FIG. 6 shows cytokine levels of IL-2, GM-CSF and TNF-α in co-cultures of CAR T cells with DLL3⁺ 293 cells. 4H11 CAR T cells were co-cultured with 3T3 cells expressing the 4H11 target MUC16.

FIGS. 7A-7F illustrate the effects of IL-18 on the activation of genetically engineered and endogenous T cells. FIG. 7A illustrates a schematic of CAR design with mCherry, SC16.8 scFv, murine CD28 and CD3z signaling domains, and the murine IL-18 transgene. FIG. 7B depicts flow cytometric analysis of murine CAR T cells for mCherry and CAR-Flag expression. FIG. 7C shows the survival of mSCLC tumor-bearing mice after treatment with mCherry-expressing CAR T cells that do or do not secrete mIL-18, compared to the original mCherry-negative CAR T cells. FIGS. 7D and 7E show IFNg ELISPOT results, quantified in FIG. 3F, comparing mCherry-negative endogenous CD4 and CD8 tumor-infiltrating lymphocytes from mice treated with SC16.8 or SC16.8_mIL18 CAR T cells, harvested from mSCLC-bearing livers 3 (FIG. 7D) or 6 (FIG. 7E) days after CAR T cell treatment. mCherry⁺ CAR T cells were isolated and included as positive control. FIG. 7F illustrates DLL3 surface expression on DLL3-negative Jurkat cells, DLL3-positive mSCLC cells and DLL3-knockout mSCLC cells.

FIGS. 8A-8F illustrate complete flow cytometric analysis of myeloid cells on days 3, 6 and 10 post CAR T cell treatment in mSCLC-bearing mice, corresponding to FIG. 4. FIG. 8A depicts levels of CD206⁺MHC-II^{lo} M2-like macrophages in livers and spleens. FIG. 8B shows expression of CD86 on macrophages in livers and spleens. FIG. 8C depicts expression of CD86 on dendritic cells in livers and spleens. FIG. 8D depicts expression of PD-L1 on macrophages in livers and spleens. FIG. 8E depicts expression of PD-L1 on dendritic cells in livers and spleens. FIG. 8F depicts expression of PD-L1, MHC-II and CD86 on F4/80⁺ macrophages in livers.

FIGS. 9A-9C Illustrate the efficacy of low-dose SC16.8_IL18 CAR T cells in SCLC models. FIG. 9A depicts tumor growth and overall survival of mice with metastatic H82 tumors that received 0.1×10⁶ CAR T cells. FIG. 9B shows tumor growth and overall survival of mice with metastatic H69 tumors that received 0.2×10⁶ CAR T cells. FIG. 9C depicts tumor growth and overall survival of mice with orthotopic SHP-77 tumors that received 0.2×10⁶ CAR T cells.

### DETAILED DESCRIPTION OF THE INVENTION

The presently disclosed subject matter provides antigen-recognizing receptors (e.g., chimeric antigen receptors (CARs)) that specifically target DLL3. The presently disclosed subject matter further provides cells comprising such receptors. In certain embodiments, the presently disclosed cells are further engineered to express an additional protein alongside the antigen-recognizing receptor (e.g., a cytokine, e.g., a IL-18 polypeptide). The cells can be immunoresponsive cells, e.g., genetically modified immunoresponsive cells (e.g., T cells or NK cells). The presently disclosed subject matter also provides methods of using such cells for treatments, e.g., for treating and or ameliorating a disease or disorder associated with DLL3.

Non-limiting embodiments of the present disclosure are described by the present specification and Examples. For purposes of clarity of disclosure and not by way of limitation, the detailed description is divided into the following subsections:
1. Definitions;
2. DLL3;
3. Antigen-Recognizing Receptors;
4. Cells;
5. Compositions and Vectors;
6. Polypeptides;
7. Formulations and Administration;
8. Methods of Treatment; and
9. Kits.

### 1. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an exogenous CAR binds to an antigen, a formation of an immunological synapse occurs that includes clustering of many molecules near the bound receptor (e.g. CD4 or CD8, CD3 □/□/□/□, etc.). This clustering of membrane bound signaling molecules allows for ITAM motifs contained within the CD3 chains to become phosphorylated. This phosphorylation in turn initiates a T cell activation pathway ultimately activating transcription factors, such as NF-□B and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell. By “soluble” is meant a polypeptide that is freely diffusible in an aqueous environment (e.g., not membrane bound).

The term “antigen-recognizing receptor” as used herein refers to a receptor that is capable of recognizing a target antigen (e.g., DLL3). In certain embodiments, the antigen-recognizing receptor is capable of activating an immune or immunoresponsive cell (e.g., a T cell) upon its binding to the target antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., Nucl Med (1983); 24:316-325). As used herein, include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the IMGT numbering system. In certain embodiments, the CDR regions are delineated using the IMGT numbering system accessible at http://www.imgt.org/IMGT_vquest/input.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. In certain embodiments, the linker is a G4S linker.

In certain embodiments, the linker comprises or consists of the amino acid sequence set forth in SEQ ID NO: 78, which is provided below:

[SEQ ID NO: 78]
GGGGSGGGGSGGGSGGGGS

In certain embodiments, the linker comprise or consists of the amino acid sequence set forth in SEQ ID NO: 79, which is provided below:

[SEQ ID NO: 79]
GGGGSGGGGSGGGGS

In certain embodiments, the linker comprises or consists of the amino acid sequence set forth in SEQ ID NO: 80, which is provided below:

[SEQ ID NO: 80]
GGGGSGGGGSGGGGSGGGSGGGGS

In certain embodiments, the linker comprises or consists of the amino acid sequence set forth in SEQ ID NO: 81, which is provided below:

[SEQ ID NO: 81]
GGGGSGGGGSGGGGSGGGGSGGGSGGGGS

In certain embodiments, the linker comprises or consists of the amino acid sequence set forth in SEQ ID NO: 82, which is provided below:

[SEQ ID NO: 82]
GGGGS

In certain embodiments, the linker comprises or consists of the amino acid sequence set forth in SEQ ID NO: 83, which is provided below:

[SEQ ID NO: 83]
GGGGSGGGGS

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. Proc. Nat. Acad. Sci. USA, (1988); 85:5879-5883; U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) (2008); 27(6):455-51; Peter et al., J Cachexia Sarcopenia Muscle (2012); August 12; Shieh et al., J Imunol (2009); 183(4):2277-85; Giomarelli et al., Thromb Haemost (2007); 97(6):955-63; Fife eta., J Clin Invst (2006); 116(8):2252-61; Brocks et al., Immunotechnology 1997 3(3):173-84; Moosmayer et al., Ther Immunol 1995 2(10:31-40). Agonistic scFvs having stimulatory activity have been described (Peter et al., J Biol Chem (2003); 25278(38):36740-7; Xie et al., Nat Biotech 1997 15(8):768-71; Ledbetter et al., Crit Rev Immunol (1997); 17(5-6):427-55; Ho et al., BioChim Biophys Acta (2003); 1638(3):257-66).

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. By “substantially identical” or “substantially homologous” is meant a polypeptide or nucleic acid molecule exhibiting at least about 50% homologous or identical to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

An “effective amount” is an amount sufficient to affect a beneficial or desired clinical result upon treatment. An effective amount can be administered to a subject in one or more doses. In certain embodiments, an effective amount can be an amount that is sufficient to palliate, ameliorate, stabilize, reverse or slow the progression of the disease, or otherwise reduce the pathological consequences of the disease. The effective amount can be determined by a physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

As used herein, the term “a conservative sequence modification” refers to an amino acid modification that does not significantly affect or alter the binding characteristics of the presently disclosed DLL3-targeted CAR (e.g., the extracellular antigen-binding domain) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence or a CDR region are altered.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “recognize” is meant selectively binds to a target. A T cell that recognizes a tumor can expresses a receptor (e.g., a CAR) that binds to a tumor antigen.

By “signal sequence” or “leader sequence” is meant a peptide sequence (e.g., 5, 10, 15, 20, 25 or 30 amino acids) present at the N-terminus of newly synthesized proteins that directs their entry to the secretory pathway By “specifically binds” or “specifically binds to” or “specifically target” is meant a polypeptide or a fragment thereof that recognizes and/or binds to a biological molecule of interest (e.g., a polypeptide, e.g., a DLL3 polypeptide), but which does not substantially recognize and/or bind other molecules in a sample, for example, a biological sample, which naturally includes a presently disclosed polypeptide (e.g., a DLL3 polypeptide).

As used herein, the term “derivative” refers to a compound that is derived from some other compound and maintains its general structure. For example, but without any limitation, trichloromethane (chloroform) is a derivative of methane.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system but can affect people with a poorly functioning or suppressed immune system.

Other aspects of the presently disclosed subject matter are described in the following disclosure and are within the ambit of the presently disclosed subject matter.

### 2. DLL3

DLL3 is selectively expressed in high grade pulmonary neuroendocrine tumors of the lung (LU-NETs) and other neuroendocrine cancers. Lu-NETs embrace a heterogeneous family of neoplasms classified into four histological variants, namely typical carcinoid (TC), atypical carcinoid (AC), large cell neuroendocrine carcinoma (LCNEC) and small cell lung carcinoma (SCLC). Increased expression of DLL3 was observed in SCLC and LCNEC patient-derived xenograft tumors and was also confirmed in primary tumors. See Saunders et al., Sci Translational Medicine (302): 302ra136 (2015). Both SCLC and pulmonary LCNEC are high-grade and poor-prognosis tumors, with higher incidence in smokers. Pulmonary LCNEC exhibits biologically aggressive behavior, similarly to SCLC. Stage by stage, survival curves of pulmonary LCNEC and SCLC overlap, and in addition, survival is lower than other NSCLCs. Prognosis is poor even in patients with potentially resectable stage I lung cancer with 5-year survival rates ranging from 27% to 67%. See Iyoda A. et al., J Thorac Cardiovasc Surg. 138:446-453 (2009).

Delta is one of the Drosophila ligands of Notch that activate signaling in adjacent cells. Humans have four known Notch receptors (NOTCH1 to NOTCH4), and three homologs of Delta, termed delta-like ligands: DLL1, DLL3 and DLL4. It has been reported that unlike DLL1 and DLL4, DLL3 inhibits Notch signaling rather than activating it.

DLL3 (also known as Delta-like 3 or SCDO1) is a member of the Delta-like family of Notch DSL ligands. Aberrant DLL3 expression (genotypic and/or phenotypic) is associated with various tumorigenic cell subpopulations such as cancer stem cells and tumor initiating cells.

In certain embodiments, the antigen recognizing receptor binds to human DLL3. In certain embodiments, the human DLL3 comprises or consists of the amino acid sequence with a UniProt Reference No: Q9NYJ7-1 (SEQ ID NO: 84) or a fragment thereof. SEQ ID NO: 84 is provided below. In certain embodiments, the DLL3 comprises an extracellular domain, a transmembrane domain, and a cytoplasmic domain. In certain embodiments, the extracellular domain comprises or consists of amino acids 27 to 492 of SEQ ID NO: 84. In certain embodiments, the transmembrane domain comprises or consists of amino acids 493 to 513 of SEQ ID NO: 84. In certain embodiments, the cytoplasmic domain comprises or consists of amino acids 514 to 618 of SEQ ID NO: 84.

In certain embodiments, the extracellular domain of DLL3 comprises a DSL domain, an EGF-like 1 domain, an EGF-like 2 domain, an EGF-like 3 domain, an EGF-like 4 domain, and EGF-like 5 domain, and an EGF-like 6 domain. In certain embodiments, the DSL domain comprises or consists of amino acids 176 to 215 of SEQ ID NO: 84. In certain embodiments, the EGF-like 1 domain comprises or consists of amino acids 216 to 249 of SEQ ID NO: 84. In certain embodiments, the EGF-like 2 domain comprises or consists of amino acids 274 to 310 of SEQ ID NO: 84. In certain embodiments, the EGF-like 3 domain comprises or consists of amino acids 312 to 351 of SEQ ID NO: 84. In certain embodiments, the EGF-like 4 domain comprises or consists of amino acids 353 to 389 of SEQ ID NO: 84. In certain embodiments, the EGF-like 5 domain comprises or consists of amino acids 391 to 427 of SEQ ID NO: 84. In certain embodiments, the EGF-like 6 domain comprises or consists of amino acids 429 to 465 of SEQ ID NO: 84.

[SEQ ID NO: 84]
MVSPRMSGLL SQTVILALIF LPQTRPAGVF ELQIHSFGPG
PGPGAPRSPC SARLPCRLFF RVCLKPGLSE EAAESPCALG
AALSARGPVY TEQPGAPAPD LPLPDGLLQV PFRDAWPGTF
SFIIETWREE LGDQIGGPAW SLLARVAGRR RLAAGGPWAR
DIQRAGAWEL RFSYRARCEP PAVGTACTRL CRPRSAPSRC
GPGLRPCAPL EDECEAPLVC RAGCSPEHGF CEQPGECRCL
EGWTGPLCTV PVSTSSCLSP RGPSSATTGC LVPGPGPCDG
NPCANGGSCS ETPRSFECTC PRGFYGLRCE VSGVTCADGP
CFNGGLCVGG ADPDSAYICH CPPGFQGSNC EKRVDRCSLQ
PCRNGGLCLD LGHALRCRCR AGFAGPRCEH DLDDCAGRAC
ANGGTCVEGG GAHRCSCALG FGGRDCRERA DPCAARPCAH
GGRCYAHFSG LVCACAPGYM GARCEFPVHP DGASALPAAP
PGLRPGDPQR YLLPPALGLL VAAGVAGAAL LLVHVRRRGH
SQDAGSRLLA GTPEPSVHAL PDALNNLRTQ EGSGDGPSSS
VDWNRPEDVD PQGIYVISAP SIYAREVATP LFPPLHTGRA
GQRQHLLFPY PSSILSVK

In certain embodiments, the DLL3 comprises or consists of an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 84 or a fragment thereof.

In certain embodiments, the antigen recognizing receptor binds to a portion of human DLL3. In certain embodiments, the antigen recognizing receptor binds to the extracellular domain of DLL3. In certain embodiments, the antigen recognizing receptor binds to amino acids 27 to 492 of SEQ ID NO: 84.

In certain embodiments, the antigen recognizing receptor binds to EGF-like 3 domain of DLL3. In certain embodiments, the antigen recognizing receptor binds to amino acids 312 to 351 of SEQ ID NO: 84. In certain embodiments, the antigen recognizing receptor binds to EGF-like 4 domain of DLL3. In certain embodiments, the antigen recognizing receptor binds to amino acids 353 to 389 of SEQ ID NO: 84. In certain embodiments, the antigen recognizing receptor binds to EGF-like 5 domain of DLL3. In certain embodiments, the antigen recognizing receptor binds to amino acids 391 to 427 of SEQ ID NO: 84. In certain embodiments, the antigen recognizing receptor binds to EGF-like 6 domain of DLL3. In certain embodiments, the antigen recognizing receptor binds to amino acids 429 to 465 of SEQ ID NO: 84.

### 3. Antigen-Recognizing Receptors

The presently disclosed antigen-recognizing receptors specifically target or binds to DLL3. In certain embodiments, the antigen-recognizing receptor is a chimeric antigen receptor (CAR). In certain embodiments, the antigen-recognizing receptor is a TCR like fusion molecule.

The presently disclosed subject matter also provides nucleic acid molecules that encode the presently disclosed antigen-recognizing receptors. In certain embodiments, the nucleic acid molecule comprises a nucleotide sequence that encodes a polypeptide of a DLL3-targeted antigen recognizing receptor disclosed herein.

### 3.1. Extracellular Antigen-Binding Domains

In certain embodiments, the extracellular antigen-binding domain of the antigen-recognizing receptor binds to DLL3.

In certain embodiments, the extracellular antigen-binding domain is an scFv. In certain embodiments, the scFv is a human scFv. In certain embodiments, the scFv is a humanized scFv. In certain embodiments, the scFv is a murine scFv. In certain embodiments, the scFv is identified by screening scFv phage library with an antigen-Fc fusion protein. In certain embodiments, the scFv can be derived from fusing the variable heavy and light regions of an antibody. Additional information regarding variable heavy and light regions of antibodies that bind to DLL3 can be found in International Patent Publication No. WO 2013/126746, which is incorporated by reference in its entirety.

In certain embodiments, the extracellular antigen-binding domain is a Fab. In certain embodiments, the Fab is crosslinked. In certain embodiments, the extracellular antigen-binding domain is a F(ab)₂.

Any of the foregoing molecules may be comprised in a fusion protein with a heterologous sequence to form the extracellular antigen-binding domain. In certain embodiments, the extracellular antigen-binding domain (embodied, for example, an scFv) binds to one or more DLL3 isoforms (e.g., human DLL3) with a binding affinity, for example with a dissociation constant (K_{D}) of about 1×10⁻⁷ M or less, about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, about 1×10⁻¹¹ M or less, about 5×10⁻¹² M or less, or about 1×10⁻¹² M or less. In certain embodiments, extracellular antigen-binding domain (embodied, for example, an scFv) binds to DLL3 (e.g., human DLL3) with a binding affinity, for example with a dissociation constant (K_{D}) of about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, or about 1×10⁻¹¹ M or less, about 5×10⁻¹² M or less, or about 1×10⁻¹² M or less. In certain embodiments, extracellular antigen-binding domain (embodied, for example, an scFv) binds to DLL3 (e.g., human DLL3) with a binding affinity, for example with a dissociation constant (K_{D}) of about 5×10⁻⁹ M or less. In certain embodiments, extracellular antigen-binding domain (embodied, for example, an scFv) binds to DLL3 (e.g., human DLL3) with a binding affinity, for example with a dissociation constant (K_{D}) of about 1×10⁻⁹ M or less. In certain embodiments, extracellular antigen-binding domain (embodied, for example, an scFv) binds to DLL3 (e.g., human DLL3) with a binding affinity, for example with a dissociation constant (K_{D}) of about 2×10¹⁰ M.

Binding of the extracellular antigen-binding domain can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the DLL3-targeted extracellular antigen-binding domain is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet). In certain embodiments, the DLL3-targeted human scFv is labeled with GFP.

In certain embodiments, the extracellular antigen-binding domain (e.g., an scFv) comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof. SEQ ID NOs: 1-3 are provided in Table 1. In certain embodiments, the extracellular antigen-binding domain (e.g., an scFv) comprises a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification thereof, and a CDR3 comprising the amino acid sequence set forth in SEQ ID NO: 6 or a conservative modification thereof. SEQ ID NOs: 4-6 are provided in Table 1.

In certain embodiments, the extracellular antigen-binding domain (e.g., an scFv) comprises a V_{H} comprising a CDR1 comprising the amino acid sequence set forth in SEQ ID NO: 1 or a conservative modification thereof, a CDR2 comprising the amino acid sequence set forth in SEQ ID NO: 2 or a conservative modification thereof, and a CDR3 comprising the amino acid sequence set forth in SEQ ID NO: 3 or a conservative modification thereof; and a V_{L} comprising a CDR1 comprising the amino acid sequence set forth in SEQ ID NO: 4 or a conservative modification thereof, a CDR2 comprising the amino acid sequence set forth in SEQ ID NO: 5 or a conservative modification, and a CDR3 comprising the amino acid sequence set forth in SEQ ID NO: 6 or a conservative modification thereof. In certain embodiments, the extracellular antigen-binding domain (e.g., an scFv) comprises a V_{H} comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 7. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 7. In certain embodiments, the extracellular antigen-binding domain comprises a V_{H} comprising the amino sequence set forth in SEQ ID NO: 7. An exemplary nucleotide sequence encoding the amino acid sequence of SEQ ID NO: 7 is set forth in SEQ ID NO: 9. SEQ ID NOs: 7 and 9 are provided in Table 1 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 8. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 8. In certain embodiments, the extracellular antigen-binding domain comprises a V_{L} comprising the amino sequence set forth in SEQ ID NO: 8. An exemplary nucleotide sequence encoding the amino acid sequence of SEQ ID NO: 8 is set forth in SEQ ID NO: 10.

SEQ ID NOs: 8 and 10 are provided in Table 1 below.

In certain embodiments, the extracellular antigen-binding domain (e.g., an scFv) comprises a V_{H} comprising the amino acid sequence set forth in SEQ ID NO: 7, and a V_{L} comprising the amino acid sequence set forth in SEQ ID NO: 8. In certain embodiments, the extracellular antigen-binding domain is an scFv. In certain embodiments, the scFv is designated as “SC16.8”. In certain embodiments, the V_{H} and V_{L} are linked via a linker. In certain embodiments, the linker comprises the amino acid sequence set forth in SEQ ID NO: 78, SEQ ID NO: 79, SEQ ID NO: 80, SEQ ID NO: 81, SEQ ID NO: 82, or SEQ ID NO: 83.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the variable regions within the extracellular antigen-binding domain have to be linked one after another such that at the N-terminus of the extracellular antigen-binding domain, a light chain variable region (V_{L}) is positioned. In certain embodiments, the extracellular antigen-binding domain is an scFv, the variable regions are positioned from the N- to the C-terminus: V_{L}-V_{H}.

TABLE 1
SC16.8

| CDRs | 1 | 2 | 3 |
|---|---|---|---|
| V_{H} | GYAFTNYL[SEQ ID NO: 1] | INPGTGGT [SEQ ID NO: 2] | ARSPYDYHEGAMDY |
| | | | WGQGTSVTVSS |
| | | | [SEQ ID NO: 3] |
| V_{L} | QATQDIVKN [SEQ ID NO: 4] | YAIELAE [SEQ ID NO: 5] | LQFYEFPFTFGAGTK |
| | | | LELK [SEQ ID NO: 6] |

| | |
|---|---|
| Full V_{H} | QAQLQQSGAELVRPGTSVKVSCKASGYAFTNYLIEWVKQRPGQGLEWI |
| | GVINPGTGGTNYNENFKGKATLTADKSSSTAYMQLSSLTSDDSAVYFCA |
| | RSPYDYHEGAMDYWGQGTSVTVSS [SEQ ID NO: 7] |
| Full V_{L} | EIQMTQSPSSMSASLGDRITITCQATQDIVKNLNWYQQKPGKPPSFLIYY |
| | AIELAEGVPSRFSGSGSGSDYSLTISNLESEDFADYYCLQFYEFPFTFGAG |
| | TKLELK [SEQ ID NO: 8] |
| DNA for | caggcccagctgcagcagtctggagctgagctggtaaggcctgggacttcagtgaaggtgtcctgcaaggcttct |
| Full V_{H} | ggatacgccttcactaattacttgatagagtgggtaaagcagaggcctggacagggccttgagtggattggagtgat |
| | taatcctggaactggtggtactaactacaatgagaacttcaagggcaaggcaactctgactgcagacaaatcctcca |
| | gtactgcctacatgcagctcagcagcctgacatctgatgactctgcggtctatttctgtgcaagatccccctatgatta |
| | ccacgagggtgctatggactactggggtcaaggaacctcagtcaccgtctcctca [SEQ ID NO: 9] |
| DNA for | gaaatccagatgacccagtctccatcctctatgtctgcatctctgggagacagaataaccatcacttgccaggcaact |
| Full V_{L} | caagacattgttaagaatttaaactggtatcagcagaaaccagggaaacccccttcattcctgatctattatgcaattg |
| | aactggcagaaggggtcccatcaaggttcagtggcagtgggtctgggtcagactattctctgacaatcagcaacct |
| | ggagtctgaagattttgcagactattactgtctacagttttatgagtttccgttcacgttcggtgctgggaccaagctgg |
| | agctgaaac [SEQ ID NO: 10] |

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof. SEQ ID NOs: 11-13 are provided in Table 2. In certain embodiments, the extracellular antigen-binding domain (e.g., an scFv) comprises a V_{L} comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 16 or a conservative modification thereof. SEQ ID NOs: 14-16 are provided in Table 2.

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof; and a V_{L} comprising a CDR1 comprising the amino acid sequence set forth in SEQ ID NO: 14 or a conservative modification thereof, a CDR2 comprising the amino acid sequence set forth in SEQ ID NO: 15 or a conservative modification thereof, and a CDR3 comprising the amino acid sequence set forth in SEQ ID NO: 146 or a conservative modification thereof. In certain embodiments, the extracellular antigen-binding domain (e.g., a scFv) comprises a V_{H} comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13; and a V_{L} comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 17. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 17. In certain embodiments, the extracellular antigen-binding domain comprises a V_{H} comprising the amino sequence set forth in SEQ ID NO: 17. An exemplary nucleotide sequence encoding the amino acid sequence of SEQ ID NO: 17 is set forth in SEQ ID NO: 19. SEQ ID NO: 17 and 19 are provided in Table 2 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 18. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 18. In certain embodiments, the extracellular antigen-binding domain comprises a V_{L} comprising the amino sequence set forth in SEQ ID NO: 18. An exemplary nucleotide sequence encoding the amino acid sequence of SEQ ID NO: 18 is set forth in SEQ ID NO: 20. SEQ ID NO: 16 and 20 are provided in Table 2 below.

In certain embodiments, the extracellular antigen-binding domain (e.g., an scFv) comprises a V_{H} comprising the amino acid sequence set forth in SEQ ID NO: 17, and a V_{L} comprising the amino acid sequence set forth in SEQ ID NO: 18. In certain embodiments, the extracellular antigen-binding domain is an scFv. In certain embodiments, the scFv is designated as “SC16.13”. In certain embodiments, the V_{H} and V_{L} are linked via a linker. In certain embodiments, the linker comprises the amino acid sequence set forth in SEQ ID NO: 78, SEQ ID NO: 79, SEQ ID NO: 80, SEQ ID NO: 81, SEQ ID NO: 82, or SEQ ID NO: 83.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 2
SC16.13

| CDRs | 1 | 2 | 3 |
|---|---|---|---|
| V_{H} | GFSLSTSGMG [SEQ ID NO: 11] | IWWDDVK [SEQ ID NO: 12] | ARIVSFDNDVVSAMD |
| | | | YWGQGTSVTVSS |
| | | | [SEQ ID NO: 13] |
| V_{L} | SASSSVSY [SEQ ID NO: 14] | LTSNLAS [SEQ ID NO: 15] | QQWRSNPFTFGSGTK |
| | | | LEIK [SEQ ID NO: 16] |

| | |
|---|---|
| Full V_{H} | QVTLKESGPGILQPSQTLSLTCSFSGFSLSTSGMGVGWIRQPSGKGLEWL |
| | AHIWWDDVKRYNPALKSRLTISKDTSSSQVFLKIASVDTADTATYYCARI |
| | VSFDNDVVSAMDYWGQGTSVTVSS [SEQ ID NO: 17] |
| Full V_{L} | QIVLTQSPALVSASPGEKVTMTCSASSSVSYMYWYQQKPRSSPKPWIYL |
| | TSNLASGVPARFSGSGSGTSYSLTISSMEAEDAATYYCQQWRSNPFTFGS |
| | GTKLEIK [SEQ ID NO: 18] |
| DNA for | caggttactctgaaagagtctggccctgggatattgcagccctcccagaccctcagtctgacttgttctttctctgggtt |
| Full V_{H} | ttcactgagcacttctggtatgggtgtaggctggattcggcagccatcagggaagggtctggagtggctggcacac |
| | atttggtgggatgatgtcaagcgctataacccagccctgaagagccgactgactatctccaaggatacctccagca |
| | gccaggtattcctcaagatcgccagtgtggacactgcagatactgccacatactactgtgctcgcatagtttcctttga |
| | taacgacgttgtctctgctatggactactggggtcaaggaacctcagtcaccgtctcctc [SEQ ID NO: 19] |
| DNA for | caaattgttctcacccagtctccagcactcgtgtctgcatctccaggggagaaggtcaccatgacctgcagtgccag |
| Full V_{L} | ctcaagtgtaagttacatgtactggtaccagcagaaaccaagatcctcccccaaaccctggatttatctcacatccaa |
| | cctggcttctggagtccctgctcgcttcagtggcagtgggtctgggacctcttactctctcacaatcagcagcatgga |
| | ggctgaagatgctgccacttattactgccagcagtggcgtagtaacccattcacgttcggctcggggacaaagttgg |
| | aaataaaac [SEQ ID NO: 20] |

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification thereof. SEQ ID NOs: 21-23 are provided in Table 3. In certain embodiments, the extracellular antigen-binding domain (e.g., an scFv) comprises a V_{L} comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set forth in SEQ ID NO: 25 or a conservative modification thereof. SEQ ID NOs: 24-26 are provided in Table 3.

In certain embodiments, the extracellular antigen-binding domain (e.g., an scFv) comprises a V_{H} comprising a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification thereof, and a V_{L} comprising a CDR1 comprising the amino acid sequence set forth in SEQ ID NO: 24 or a conservative modification thereof, a CDR2 comprising the amino acid sequence set forth in SEQ ID NO: 25 or a conservative modification thereof, and a CDR3 comprising the amino acid sequence set forth in SEQ ID NO: 26 or a conservative modification thereof. In certain embodiments, the extracellular antigen-binding domain (e.g., an scFv) comprises a V_{H} comprising a CDR1 comprising the amino acid sequence set forth in SEQ ID NO: 21, a CDR2 comprising the amino acid sequence set forth in SEQ ID NO: 22, a CDR3 comprising the amino acid sequence set forth in SEQ ID NO: 23; and a V_{L} comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 27. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 27. In certain embodiments, the extracellular antigen-binding domain comprises a V_{H} comprising the amino sequence set forth in SEQ ID NO: 27. An exemplary nucleotide sequence encoding the amino acid sequence of SEQ ID NO: 27 is set forth in SEQ ID NO: 29. SEQ ID NO: 27 and 29 are provided in Table 3 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 28. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 28. In certain embodiments, the extracellular antigen-binding domain comprises a V_{L} comprising the amino sequence set forth in SEQ ID NO: 28. An exemplary nucleotide sequence encoding the amino acid sequence of SEQ ID NO: 28 is set forth in SEQ ID NO: 30. SEQ ID NO: 28 and 30 are provided in Table 3 below.

In certain embodiments, the extracellular antigen-binding domain (e.g., an scFv) comprises a V_{H} comprising the amino acid sequence set forth in SEQ ID NO: 27, and a V_{L} comprising the amino acid sequence set forth in SEQ ID NO: 28. In certain embodiments, the extracellular antigen-binding domain is an scFv. In certain embodiments, the scFv is designated as “SC16.15”. In certain embodiments, the V_{H} and V_{L} are linked via a linker. In certain embodiments, the linker comprises the amino acid sequence set forth in SEQ ID NO: 78, SEQ ID NO: 79, SEQ ID NO: 80, SEQ ID NO: 81, SEQ ID NO: 82, or SEQ ID NO: 83.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 3
SC16.15

| CDRs | 1 | 2 | 3 |
|---|---|---|---|
| V_{H} | SGYTFTRYW [SEQ ID NO: 21] | INPTTVYT [SEQ ID NO: 22] | ARGGSNFFDYWGQG |
| | | | TTLTVSS [SEQ ID NO: 23] |
| V_{L} | RASENIYYN [SEQ ID NO: 24] | TANSLED [SEQ ID NO: 25] | KQAYDVPPTFGGGTKLEIK |
| | | | [SEQ ID NO: 26] |

| | |
|---|---|
| Full V_{H} | QVQLQQSGAELAKPGASVKMSCKASGYTFTRYWIHWIKQRPGQGLEWI |
| | GYINPTTVYTEFNQNFKDKATLTADKSSTTASMQLSSLTSEDSAVYYCA |
| | RGGSNFFDYWGQGTTLTVSS [SEQ ID NO: 27] |
| Full V_{L} | DIQMTQSPASLAASVGETVAITCRASENIYYNLAWYQQKQGKSPQLLIY |
| | TANSLEDGVPSRFSGSGSGTQYSLKINSMQPEDSATYFCKQAYDVPPTFG |
| | GGTKLEIK [SEQ ID NO: 28] |
| DNA for | caggtccagcttcagcagtctggggctgaactggcaaaacctggggcctcagtgaagatgtcctgtaaggcttctg |
| Full V_{H} | gctacacctttactcgctactggatacactggataaaacagaggcctggacagggtctggaatggattggatacatt |
| | aatcctacaactgtttatactgagttcaatcagaacttcaaggacaaggccactttgactgcagacaaatcctccacca |
| | cagcctccatgcaactgagcagcctgacatctgaggactctgcagtctattactgtgcaagaggcggtagtaacttct |
| | ttgactactggggccaaggcaccactctcacagtctcctca [SEQ ID NO: 29] |
| DNA for | gacatccagatgactcagtctccagcctccctggctgcatctgtgggagaaactgtcgccatcacatgtcgagcaa |
| Full V_{L} | gtgagaacatttactacaatttagcatggtatcagcagaaacaagggaaatctcctcagctcctgatctatactgcaaa |
| | cagtttggaagatggtgtcccatcgaggttcagtggcagtggatctgggacacagtattctttgaagatcaacagcat |
| | gcagcctgaagattccgcaacttatttctgtaaacaggcttatgacgttcctccgacgttcggtggaggcaccaagct |
| | ggaaatcaaac [SEQ ID NO: 30] |

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 31 or a conservative modification thereof, and a CDR3 comprising the amino acid sequence set forth in SEQ ID NO: 32 or a conservative modification thereof. SEQ ID NOs: 11, 31, and 32 are provided in Table 4. In certain embodiments, the extracellular antigen-binding domain (e.g., an scFv) comprises a V_{L} comprising a CDR1 comprising the amino acid sequence set forth in SEQ ID NO: 25 or a conservative modification thereof, a CDR2 comprising the amino acid sequence set forth in SEQ ID NO: 33 or a conservative modification thereof, and a CDR3 comprising the amino acid sequence set forth in SEQ ID NO: 34 or a conservative modification thereof. SEQ ID NOs: 25, 33, and 34 are provided in Table 4.

In certain embodiments, the extracellular antigen-binding domain (e.g., an scFv) comprises a V_{H} comprising a CDR1 comprising the amino acid sequence set forth in SEQ ID NO: 11 or a conservative modification thereof, a CDR2 comprising the amino acid sequence set forth in SEQ ID NO: 31 or a conservative modification thereof, and a CDR3 comprising the amino acid sequence set forth in SEQ ID NO: 32 or a conservative modification thereof, and a V_{L} comprising a CDR1 comprising the amino acid sequence set forth in SEQ ID NO: 25 or a conservative modification thereof, a CDR2 comprising the amino acid sequence set forth in SEQ ID NO: 33 or a conservative modification thereof, and a CDR3 comprising the amino acid sequence set forth in SEQ ID NO: 34 or a conservative modification thereof. In certain embodiments, the extracellular antigen-binding domain (e.g., an scFv) comprises a V_{H} comprising a CDR1 comprising the amino acid sequence set forth in SEQ ID NO: 11, a CDR2 comprising the amino acid sequence set forth in SEQ ID NO: 31, and a CDR3 comprising the amino acid sequence set forth in SEQ ID NO: 32; and a V_{L} comprising a CDR1 comprising the amino acid sequence set forth in SEQ ID NO: 25, a CDR2 comprising the amino acid sequence set forth in SEQ ID NO: 33, and a CDR3 comprising the amino acid sequence set forth in SEQ ID NO: 34.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 35. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 35. In certain embodiments, the extracellular antigen-binding domain comprises a V_{H} comprising the amino sequence set forth in SEQ ID NO: 35. An exemplary nucleotide sequence encoding the amino acid sequence of SEQ ID NO: 35 is set forth in SEQ ID NO: 37. SEQ ID NO: 35 and 37 are provided in Table 4 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 36. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 36. In certain embodiments, the extracellular antigen-binding domain comprises a V_{L} comprising the amino sequence set forth in SEQ ID NO: 36. An exemplary nucleotide sequence encoding the amino acid sequence of SEQ ID NO: 36 is set forth in SEQ ID NO: 38. SEQ ID NO: 36 and 38 are provided in Table 4 below.

In certain embodiments, the extracellular antigen-binding domain (e.g., an scFv) comprises a V_{H} comprising the amino acid sequence set forth in SEQ ID NO: 35, and a V_{L} comprising the amino acid sequence set forth in SEQ ID NO: 36. In certain embodiments, the extracellular antigen-binding domain is an scFv. In certain embodiments, the scFv is designated as “SC16.25”. In certain embodiments, the V_{H} and V_{L} are linked via a linker. In certain embodiments, the linker comprises the amino acid sequence set forth in SEQ ID NO: 78, SEQ ID NO: 79, SEQ ID NO: 80, SEQ ID NO: 81, SEQ ID NO: 82, or SEQ ID NO: 83.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 4
SC16.25

| CDRs | 1 | 2 | 3 |
|---|---|---|---|
| V_{H} | GFSLSTSGMG [SEQ ID NO: 11] | IWWDDNK [SEQ ID NO: 31] | ARRVNYYYDPYYAM |
| | | | DYWGQGTSVTVSS |
| | | | [SEQ ID NO: 32] |
| V_{L} | SASSSVSY [SEQ ID NO: 14] | DSSKLAS [SEQ ID NO: 33] | QQWSSNPLTFGAGTKLELK |
| | | | [SEQ ID NO: 34] |

| | |
|---|---|
| Full V_{H} | QVTLKESGPGILQPSQTLSLTCSFSGFSLSTSGMGVGWIRQPSGEGLEWL |
| | TDIWWDDNKYYNPSLKSRLTISKDTSSNQVFLNITSVDTADTATYYCAR |
| | RVNYYYDPYYAMDYWGQGTSVTVSS [SEQ ID NO: 35] |
| Full V_{L} | QIVLTQSPAIMSASPGEKVTMTCSASSSVSYMHWYQQKSGTSPKRWIYD |
| | SSKLASGVPARFSGSGSGTSYSLTISSMEAEDAATYYCQQWSSNPLTFGA |
| | GTKLELK [SEQ ID NO: 36] |
| DNA for | caggttactctgaaagagtctggccctgggatattgcagccctcccagaccctcagtctgacttgttctttctctgggtt |
| Full V_{H} | ttcactgagcacttctggtatgggtgtaggctggattcgtcagccttcaggagagggtctagagtggctgacagaca |
| | tttggtgggatgacaataagtactataacccatccctgaagagccggctcacaatctccaaggatacctccagcaac |
| | caggtattcctcaatatcaccagtgtggacactgcagatactgccacttactactgtgctcgaagagttaactattatta |
| | cgacccgtactatgctatggactactggggtcaaggaacctcagtcaccgtctcctca [SEQ ID NO: 37] |
| DNA for | caaattgttctcacccagtctccagcaatcatgtctgcatctccaggggagaaggtcaccatgacctgcagtgccag |
| Full V_{L} | ctcaagtgtaagttacatgcactggtaccagcagaagtcaggcacctcccccaaaagatggatttatgactcatcca |
| | aactggcttctggagtccctgctcgcttcagtggcagtgggtctgggacctcttactctctcacaatcagcagcatgg |
| | aggctgaagatgctgccacttattactgccagcagtggagtagtaacccgctcacgttcggtgctgggaccaagct |
| | ggagctgaaac [SEQ ID NO: 38] |

In certain embodiments, the extracellular antigen-binding domain (e.g., an scFv) comprises a V_{H} comprising a CDR1 comprising the amino acid sequence set forth in SEQ ID NO: 39 or a conservative modification thereof, a CDR2 comprising the amino acid sequence set forth in SEQ ID NO: 40 or a conservative modification thereof, and a CDR3 comprising the amino acid sequence set forth in SEQ ID NO: 41 or a conservative modification thereof. SEQ ID NOs: 39-41 are provided in Table 5. In certain embodiments, the extracellular antigen-binding domain (e.g., an scFv) comprises a V_{L} comprising a CDR1 comprising the amino acid sequence set forth in SEQ ID NO: 42 or a conservative modification thereof, a CDR2 comprising the amino acid sequence set forth in SEQ ID NO: 43 or a conservative modification thereof, and a CDR3 comprising the amino acid sequence set forth in SEQ ID NO: 44 or a conservative modification thereof. SEQ ID NOs: 42-43 are provided in Table 5.

In certain embodiments, the extracellular antigen-binding domain (e.g., an scFv) comprises a V_{H} comprising a CDR1 comprising the amino acid sequence set forth in SEQ ID NO: 39 or a conservative modification thereof, a CDR2 comprising the amino acid sequence set forth in SEQ ID NO: 40 or a conservative modification thereof, and a CDR3 comprising the amino acid sequence set forth in SEQ ID NO: 41 or a conservative modification thereof, and a V_{L} comprising a CDR1 comprising the amino acid sequence set forth in SEQ ID NO: 42 or a conservative modification thereof, a CDR2 comprising the amino acid sequence set forth in SEQ ID NO: 43 or a conservative modification thereof, and a CDR3 comprising the amino acid sequence set forth in SEQ ID NO: 44 or a conservative modification thereof. In certain embodiments, the extracellular antigen-binding domain (e.g., an scFv) comprises a V_{H} comprising a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41; and a V_{L} comprising a CDR1 comprising the amino acid sequence set forth in SEQ ID NO: 42, a CDR2 comprising the amino acid sequence set forth in SEQ ID NO: 43, and a CDR3 comprising the amino acid sequence set forth in SEQ ID NO: 44.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 45. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 45. In certain embodiments, the extracellular antigen-binding domain comprises a V_{H} comprising the amino sequence set forth in SEQ ID NO: 45. An exemplary nucleotide sequence encoding the amino acid sequence of SEQ ID NO: 45 is set forth in SEQ ID NO: 47. SEQ ID NO: 45 and 47 are provided in Table 5 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 46. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 46. In certain embodiments, the extracellular antigen-binding domain comprises a V_{L} comprising the amino sequence set forth in SEQ ID NO: 46. An exemplary nucleotide sequence encoding the amino acid sequence of SEQ ID NO: 46 is set forth in SEQ ID NO: 48. SEQ ID NO: 46 and 48 are provided in Table 5 below.

In certain embodiments, the extracellular antigen-binding domain (e.g., an scFv) comprises a V_{H} comprising the amino acid sequence set forth in SEQ ID NO: 45, and a V_{L} comprising the amino acid sequence set forth in SEQ ID NO: 46. In certain embodiments, the extracellular antigen-binding domain is an scFv. In certain embodiments, the scFv is designated as “SC16.67”. In certain embodiments, the V_{H} and V_{L} are linked via a linker. In certain embodiments, the linker comprises the amino acid sequence set forth in SEQ ID NO: 78, SEQ ID NO: 79, SEQ ID NO: 80, SEQ ID NO: 81, SEQ ID NO: 82, or SEQ ID NO: 83.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 5
SC16.67

| CDRs | 1 | 2 | 3 |
|---|---|---|---|
| V_{H} | AFTFTTYA [SEQ ID NO: 39] | IRNKSNNYAT [SEQ ID NO: 40] | VFYYDYVYWGQGTLVTVSA |
| | | | [SEQ ID NO: 41] |
| V_{L} | RSSTGAVTTSNY [SEQ ID NO: 42] | GTNNRAP [SEQ ID NO: 43] | GLWYSNHLVFGGGTKLTVL |
| | | | [SEQ ID NO: 44] |

| | |
|---|---|
| Full V_{H} | EVQLVETGGGLVQPKGSLKLSCAVSAFTFTTYAMNWVRQAPGKGLEW |
| | VARIRNKSNNYATYYADSVKDRFTISRDDSQSMLYLQMNNLKIEDTAM |
| | YYCVFYYDYVYWGQGTLVTVSA [SEQ ID NO: 45] |
| Full V_{L} | QAVVTQESALTTSPGETVTLTCRSSTGAVTTSNYANWIQEKPDHLFTGLI |
| | GGTNNRAPGVPARFSGSLIGDKAALTITGAQTEDEAIYFCGLWYSNHLV |
| | FGGGTKLTVL [SEQ ID NO: 46] |
| DNA for | gaggtgcagcttgttgagactggtggaggattggtgcagcctaaagggtcattgaaactctcatgtgcagtctctgc |
| Full V_{H} | attcaccttcactacctacgccatgaactgggtccgccaggctccaggaaagggtttggagtgggttgctcgcataa |
| | gaaataaaagtaataattatgcaacatattatgccgattcagtgaaagacaggttcaccatctccagagatgattcaca |
| | aagcatgctctatctgcaaatgaacaacttgaaaattgaggacacagccatgtattactgtgtgttctactatgattacg |
| | tctactggggccaagggactctggtcactgtctctgca [SEQ ID NO: 47] |
| DNA for | caggctgttgtgactcaggaatctgcactcaccacatcacctggtgaaacagtcacactcacttgtcgctcaagtact |
| Full V_{L} | ggggctgttacaactagtaactatgccaactggatccaagaaaaaccagatcatttattcactggtctaataggtggta |
| | ccaacaaccgagctccaggtgttcctgccagattctcaggctccctgattggagacaaggctgccctcaccatcac |
| | aggggcacagactgaggatgaggcaatatatttctgtggtctatggtacagcaaccatttggtgttcggtggaggaa |
| | ccaaactgactgtcctag [SEQ ID NO: 48] |

In certain embodiments, the extracellular antigen-binding domain (e.g., an scFv) comprises a V_{H} comprising a CDR1 comprising the amino acid sequence set forth in SEQ ID NO: 49 or a conservative modification thereof, a CDR2 comprising the amino acid sequence set forth in SEQ ID NO: 50 or a conservative modification thereof, and a CDR3 comprising the amino acid sequence set forth in SEQ ID NO: 51 or a conservative modification thereof. SEQ ID NOs: 49-51 are provided in Table 6. In certain embodiments, the extracellular antigen-binding domain (e.g., an scFv) comprises a V_{L} comprising a CDR1 comprising the amino acid sequence set forth in SEQ ID NO: 52 or a conservative modification thereof, a CDR2 comprising the amino acid sequence set forth in SEQ ID NO: 53 or a conservative modification thereof, and a CDR3 comprising the amino acid sequence set forth in SEQ ID NO: 54 or a conservative modification thereof. SEQ ID NOs: 52-54 are provided in Table 6.

In certain embodiments, the extracellular antigen-binding domain (e.g., an scFv) comprises a V_{H} comprising a CDR1 comprising the amino acid sequence set forth in SEQ ID NO: 52 or a conservative modification thereof, a CDR2 comprising the amino acid sequence set forth in SEQ ID NO: 53 or a conservative modification thereof, and a CDR3 comprising the amino acid sequence set forth in SEQ ID NO: 54 or a conservative modification thereof, and a V_{L} comprising a CDR1 comprising the amino acid sequence set forth in SEQ ID NO: 52 or a conservative modification thereof, a CDR2 comprising the amino acid sequence set forth in SEQ ID NO: 53 or a conservative modification thereof, and a CDR3 comprising the amino acid sequence set forth in SEQ ID NO: 54 or a conservative modification thereof. In certain embodiments, the extracellular antigen-binding domain (e.g., an scFv) comprises a V_{H} comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51; and a V_{L} comprising a CDR1 comprising the amino acid sequence set forth in SEQ ID NO: 52, a CDR2 comprising the amino acid sequence set forth in SEQ ID NO: 53, and a CDR3 comprising the amino acid sequence set forth in SEQ ID NO: 54.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 55. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 55. In certain embodiments, the extracellular antigen-binding domain comprises a V_{H} comprising the amino sequence set forth in SEQ ID NO: 55. An exemplary nucleotide sequence encoding the amino acid sequence of SEQ ID NO: 55 is set forth in SEQ ID NO: 57. SEQ ID NO: 55 and 57 are provided in Table 6 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 56. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 56. In certain embodiments, the extracellular antigen-binding domain comprises a V_{L} comprising the amino sequence set forth in SEQ ID NO: 56. An exemplary nucleotide sequence encoding the amino acid sequence of SEQ ID NO: 56 is set forth in SEQ ID NO: 58. SEQ ID NO: 56 and 58 are provided in Table 6 below.

In certain embodiments, the extracellular antigen-binding domain (e.g., an scFv) comprises a V_{H} comprising the amino acid sequence set forth in SEQ ID NO: 55, and a V_{L} comprising the amino acid sequence set forth in SEQ ID NO: 56. In certain embodiments, the extracellular antigen-binding domain is an scFv. In certain embodiments, the scFv is designated as “SC16.118”. In certain embodiments, the linker comprises the amino acid sequence set forth in SEQ ID NO: 78, SEQ ID NO: 79, SEQ ID NO: 80, SEQ ID NO: 81, SEQ ID NO: 82, or SEQ ID NO: 83.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 6
SC16.118

| CDRs | 1 | 2 | 3 |
|---|---|---|---|
| V_{H} | GYSFTGYY [SEQ ID NO: 49] | VNPNNGGT [SEQ ID NO: 50] | ARGSYDYAEGWGQGTLVTVSA |
| | | | [SEQ ID NO: 51] |
| V_{L} | KASQSVDYDGDSY | AASNLES [SEQ ID NO: 53] | QQSNEDPYTFGGGTKLEIKR |
| | [SEQ ID NO: 52] | | [SEQ ID NO: 54] |

| | |
|---|---|
| Full V_{H} | EVQLQQSGPDLVKPGASVKISCKASGYSFTGYYMHWVKQSHGKSLEWI |
| | GRVNPNNGGTSYNQKFKGKAILTADKSSSTAYMELRSLTSEDSAVYYCA |
| | RGSYDYAEGWGQGTLVTVSA [SEQ ID NO: 55] |
| Full V_{L} | DIVLTQSPASLAVSLGQRATISCKASQSVDYDGDSYLTWYQQKPGQPPK |
| | LLIYAASNLESGIPARFSGSGSGTDFTLNIHPVEEEDAATYYCQQSNEDPY |
| | TFGGGTKLEIKR [SEQ ID NO: 56] |
| DNA for | gaggtccagctgcagcagtctggacctgacctggtgaagcctggggcttcagtgaagatatcctgcaaggcttctg |
| Full V_{H} | gttactcattcactggctactacatgcactgggtgaagcagagccatggaaagagccttgagtggattggacgtgtt |
| | aatcctaacaatggtggtactagctacaaccagaagttcaagggcaaggccatattaactgcagacaagtcatcca |
| | gcacagcctacatggagctccgcagcctgacatctgaggactctgcggtctattactgtgcaagagggagttatgat |
| | tacgccgagggctggggccaagggactctggtcactgtctctgca [SEQ ID NO: 57] |
| DNA for | gacattgtgctgacccaatctccagcttctttggctgtgtctctagggcagagggccaccatctcctgcaaggccag |
| Full V_{L} | ccaaagtgttgattatgatggtgatagttatttgacctggtaccaacagaaaccaggacagccacccaaactcctcat |
| | ctatgctgcatccaatctagaatctgggatcccagccaggtttagtggcagtgggtctgggacagacttcaccctca |
| | acatccatcctgtggaggaggaggacgctgcaacctattactgtcagcaaagtaatgaggatccgtacacgttcgg |
| | aggggggaccaagctggaaataaaacg [SEQ ID NO: 58] |

In certain embodiments, the extracellular antigen-binding domain (e.g., an scFv) comprises a V_{H} comprising a CDR1 comprising the amino acid sequence set forth in SEQ ID NO: 59 or a conservative modification thereof, a CDR2 comprising the amino acid sequence set forth in SEQ ID NO: 60 or a conservative modification thereof, and a CDR3 comprising the amino acid sequence set forth in SEQ ID NO: 61 or a conservative modification thereof. SEQ ID NOs: 59-61 are provided in Table 7. In certain embodiments, the extracellular antigen-binding domain (e.g., an scFv) comprises a V_{L} Comprising a CDR1 comprising the amino acid sequence set forth in SEQ ID NO: 62 or a conservative modification thereof, a CDR2 comprising the amino acid sequence set forth in SEQ ID NO: 63 or a conservative modification thereof, and a CDR3 comprising the amino acid sequence set forth in SEQ ID NO: 64 or a conservative modification thereof. SEQ ID NOs: 62-64 are provided in Table 7.

In certain embodiments, the extracellular antigen-binding domain (e.g., an scFv) comprises a V_{H} comprising a CDR1 comprising the amino acid sequence set forth in SEQ ID NO: 59 or a conservative modification thereof, a CDR2 comprising the amino acid sequence set forth in SEQ ID NO: 60 or a conservative modification thereof, and a CDR3 comprising the amino acid sequence set forth in SEQ ID NO: 61 or a conservative modification thereof, and a V_{L} comprising a CDR1 comprising the amino acid sequence set forth in SEQ ID NO: 62 or a conservative modification thereof, a CDR2 comprising the amino acid sequence set forth in SEQ ID NO: 63 or a conservative modification thereof, and a CDR3 comprising the amino acid sequence set forth in SEQ ID NO: 64 or a conservative modification thereof. In certain embodiments, the extracellular antigen-binding domain (e.g., an scFv) comprises a V_{H} comprising a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61; and a V_{L} comprising a CDR1 comprising the amino acid sequence set forth in SEQ ID NO: 62, a CDR2 comprising the amino acid sequence set forth in SEQ ID NO: 63, and a CDR3 comprising the amino acid sequence set forth in SEQ ID NO: 64.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 65. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 65. In certain embodiments, the extracellular antigen-binding domain comprises a V_{H} comprising the amino sequence set forth in SEQ ID NO: 65. An exemplary nucleotide sequence encoding the amino acid sequence of SEQ ID NO: 65 is set forth in SEQ ID NO: 67. SEQ ID NO: 65 and 67 are provided in Table 7 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 66. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 66. In certain embodiments, the extracellular antigen-binding domain comprises a V_{L} comprising the amino sequence set forth in SEQ ID NO: 66. An exemplary nucleotide sequence encoding the amino acid sequence of SEQ ID NO: 66 is set forth in SEQ ID NO: 68. SEQ ID NO: 66 and 68 are provided in Table 7 below.

In certain embodiments, the extracellular antigen-binding domain (e.g., an scFv) comprises a V_{H} comprising the amino acid sequence set forth in SEQ ID NO: 65, and a V_{L} comprising the amino acid sequence set forth in SEQ ID NO: 66. In certain embodiments, the extracellular antigen-binding domain is an scFv. In certain embodiments, the scFv is designated as “SC16.125”. In certain embodiments, the V_{H} and V_{L} are linked via a linker. In certain embodiments, the linker comprises the amino acid sequence set forth in SEQ ID NO: 78, SEQ ID NO: 79, SEQ ID NO: 80, SEQ ID NO: 81, SEQ ID NO: 82, or SEQ ID NO: 83.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 7
SC16.125

| CDRs | 1 | 2 | 3 |
|---|---|---|---|
| V_{H} | GYSITSGYS [SEQ ID NO: 59] | IHYSGST [SEQ ID NO: 60] | ALEGNYDGFAYWGQGTLVTVS |
| | | | [SEQ ID NO: 61] |
| V_{L} | HASQNINVW [SEQ ID NO: 62] | KASILHT [SEQ ID NO: 63] | QQGQSYPYTFGGGTKLEIK |
| | | | [SEQ ID NO: 64] |

| | |
|---|---|
| Full V_{H} | SDVQLQESGPDLVKPSQSLSLTCTVTGYSITSGYSWHWIRQFPGNKLEW |
| | MGYIHYSGSTNYNPSLKSRISITRDTSKNQFFLQFKSVTTEDSATYYCALE |
| | GNYDGFAYWGQGTLVTVS [SEQ ID NO: 65] |
| Full V_{L} | DIQMNQSPSSLSASLGDTITITCHASQNINVWLSWYQQKPGNIPKLLIYKA |
| | SILHTGVPSRFSGSGSGTGFTLTISSLQPEDIATYSCQQGQSYPYTFGGGTK |
| | LEIK [SEQ ID NO: 66] |
| DNA for | tctgatgtgcagcttcaggagtcaggacctgacctggtgaaaccttctcagtcactttcactcacctgcactgtcactg |
| Full V_{H} | gctactccatcaccagtggttatagctggcactggatccggcagtttccaggaaacaaactggaatggatgggctac |
| | atacactacagtggtagcactaactacaacccatctctcaaaagtcgaatctctatcactcgagacacatccaagaac |
| | cagttcttcctgcagttcaaatctgtgactactgaagactcagccacatattactgtgccctagaggggaattacgacg |
| | ggtttgcttactggggccaagggactctggtcactgtctctg [SEQ ID NO: 67] |
| DNA for | gacatccagatgaaccagtctccatccagtctgtctgcatcccttggagacacaattaccatcacttgccatgccagt |
| Full V_{L} | cagaacattaatgtttggttaagctggtaccagcagaaaccaggaaatattcctaaactattgatctataaggcttccat |
| | cttacacacaggcgtcccatcaaggtttagtggcagtggatctggaacaggtttcacattaaccatcagcagcctgc |
| | agcctgaagacattgccacttactcctgtcaacagggtcaaagttatccgtacacgttcggaggggggaccaagct |
| | ggaaataaaa [SEQ ID NO: 68] |

In certain embodiments, the extracellular antigen-binding domain (e.g., an scFv) comprises a V_{H} comprising a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof. SEQ ID NOs: 69-71 are provided in Table 8. In certain embodiments, the extracellular antigen-binding domain (e.g., an scFv) comprises a V_{L} comprising a CDR1 comprising the amino acid sequence set forth in SEQ ID NO: 62 or a conservative modification thereof, a CDR2 comprising the amino acid sequence set forth in SEQ ID NO: 72 or a conservative modification thereof, and a CDR3 comprising the amino acid sequence set forth in SEQ ID NO: 73 or a conservative modification thereof. SEQ ID NOs: 62, 72, and 73 are provided in Table 8.

In certain embodiments, the extracellular antigen-binding domain (e.g., an scFv) comprises a V_{H} comprising a CDR1 comprising the amino acid sequence set forth in SEQ ID NO: 69 or a conservative modification thereof, a CDR2 comprising the amino acid sequence set forth in SEQ ID NO: 70 or a conservative modification thereof, and a CDR3 comprising the amino acid sequence set forth in SEQ ID NO: 71 or a conservative modification thereof, and a V_{L} comprising a CDR1 comprising the amino acid sequence set forth in SEQ ID NO: 62 or a conservative modification thereof, a CDR2 comprising the amino acid sequence set forth in SEQ ID NO: 72 or a conservative modification thereof, and a CDR3 comprising the amino acid sequence set forth in SEQ ID NO: 73 or a conservative modification thereof. In certain embodiments, the extracellular antigen-binding domain (e.g., an scFv) comprises a V_{H} comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and a V_{L} comprising a CDR1 comprising the amino acid sequence set forth in SEQ ID NO: 62, a CDR2 comprising the amino acid sequence set forth in SEQ ID NO: 72, and a CDR3 comprising the amino acid sequence set forth in SEQ ID NO: 73.

In certain embodiments, the extracellular antigen-binding domain (e.g., an scFv) comprises a V_{H} comprising an amino acid sequence that is at least about 80% (e.g., at least about 85%, at least about 90%, or at least about 95%) homologous or identical to the amino sequence set forth in SEQ ID NO: 74. For example, the extracellular antigen-binding domain (e.g., an scFv) comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 74. In certain embodiments, the extracellular antigen-binding domain comprises a V_{H} comprising the amino sequence set forth in SEQ ID NO: 74. An exemplary nucleotide sequence encoding the amino acid sequence of SEQ ID NO: 74 is set forth in SEQ ID NO: 76. SEQ ID NO: 74 and 76 are provided in Table 8 below.

In certain embodiments, the extracellular antigen-binding domain (e.g., an scFv) comprises a V_{L} comprising an amino acid sequence that is at least about 80% (e.g., at least about 85%, at least about 90%, or at least about 95%) homologous or identical to the amino sequence set forth in SEQ ID NO: 75. For example, the extracellular antigen-binding domain (e.g., an scFv) comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to the amino sequence set forth in SEQ ID NO: 75. In certain embodiments, the extracellular antigen-binding domain comprises a V_{L} comprising the amino sequence set forth in SEQ ID NO: 75. An exemplary nucleotide sequence encoding the amino acid sequence of SEQ ID NO: 75 is set forth in SEQ ID NO: 77. SEQ ID NO: 75 and 77 are provided in Table 8 below.

In certain embodiments, the extracellular antigen-binding domain (e.g., an scFv) comprises a V_{H} comprising the amino acid sequence set forth in SEQ ID NO: 74, and a V_{L} comprising the amino acid sequence set forth in SEQ ID NO: 75. In certain embodiments, the extracellular antigen-binding domain is an scFv. In certain embodiments, the scFv is designated as “SC16.126”. In certain embodiments, the V_{H} and V_{L} are linked via a linker. In certain embodiments, the linker comprises the amino acid sequence set forth in SEQ ID NO: 78, SEQ ID NO: 79, SEQ ID NO: 80, SEQ ID NO: 81, SEQ ID NO: 82, or SEQ ID NO: 83.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}. In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

TABLE 8
SC16.126

| CDRs | 1 | 2 | 3 |
|---|---|---|---|
| V_{H} | GSSLTNYG | IWAGGST | ARDWEGWFAYWGQGTLVTVSA |
| | [SEQ ID NO: 69] | [SEQ ID NO: 70] | [SEQ ID NO: 71] |
| V_{L} | HASQNINVW | KASNLHT | QQGQSYPFTFGSGTKLEIK |
| | [SEQ ID NO: 62] | [SEQ ID NO: 72] | [SEQ ID NO: 73] |

| | |
|---|---|
| Full V_{H} | QVQMKESGPGLVAPSQSLSITCTVSGSSLTNYGVHWVRQPPGKGLEWLGVIWA |
| | GGSTNYNSALMSRLSISKDNSKSQVFLKMNSLQTDDTAMYYCARDWEGWFAY |
| | WGQGTLVTVSA [SEQ ID NO: 74] |
| Full V_{L} | DIQMNQSPSSLSASLGDTITITCHASQNINVWLSWYQQKPGNIPKLLIYKASNLHT |
| | GVPSRFSGSGSGTGFTLTISSLQPEDIATYYCQQGQSYPFTFGSGTKLEIK |
| | [SEQ ID NO: 75] |
| DNA for | caggtgcagatgaaggagtcaggacctggcctggtggcgccctcacagagcctgtccatcacttgcactgtctctgggtcttcat |
| Full V_{H} | taaccaactatggtgtacactgggttcgccagcctccaggaaagggtctagagtggctgggagtaatatgggctggtggaagc |
| | acaaattataattcggctctcatgtccagactgagtatcagcaaagacaactccaagagccaagttttcttaaaaatgaacagtctg |
| | caaactgatgacacagccatgtactactgtgccagagactgggagggctggtttgcttactggggccaagggactctggtcact |
| | gtctctgca [SEQ ID NO: 76] |
| DNA for | gacatccagatgaaccagtctccatccagtctgtctgcatcccttggagacacaattaccatcacttgccatgccagtcagaacat |
| Full V_{L} | aaatgtttggttaagctggtaccagcagaaaccaggaaatattcctaaactattgatctataaggcttccaacttgcacacaggcgt |
| | cccatcaaggtttagtggcagtggatctggaacaggtttcacattaaccatcagcagcctgcagcctgaagacattgccacttact |
| | actgtcaacagggtcaaagttatccattcacgttcggctcggggacaaagttggaaataaaac [SEQ ID NO: 77] |

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 7, SEQ ID NO: 8, SEQ ID NO: 17, SEQ ID NO: 18, SEQ ID NO: 27, SEQ ID NO: 28, SEQ ID NO: 35, SEQ ID NO: 36, SEQ ID NO: 45, SEQ ID NO: 46, SEQ ID NO: 55, SEQ ID NO: 56, SEQ ID NO: 65, SEQ ID NO: 66, SEQ ID NO: 74, or SEQ ID NO: 75) may contain substitutions (e.g., conservative substitutions), insertions, or deletions relative to the specified sequence(s), but retain the ability to bind to DLL3.

In certain embodiments, a total of 1 to 10 amino acids are substituted, inserted and/or deleted in a specific sequence (e.g., SEQ ID NO: 7, SEQ ID NO: 8, SEQ ID NO: 17, SEQ ID NO: 18, SEQ ID NO: 27, SEQ ID NO: 28, SEQ ID NO: 35, SEQ ID NO: 36, SEQ ID NO: 45, SEQ ID NO: 46, SEQ ID NO: 55, SEQ ID NO: 56, SEQ ID NO: 65, SEQ ID NO: 66, SEQ ID NO: 74, or SEQ ID NO: 75). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NO: 7, SEQ ID NO: 8, SEQ ID NO: 17, SEQ ID NO: 18, SEQ ID NO: 27, SEQ ID NO: 28, SEQ ID NO: 35, SEQ ID NO: 36, SEQ ID NO: 45, SEQ ID NO: 46, SEQ ID NO: 55, SEQ ID NO: 56, SEQ ID NO: 65, SEQ ID NO: 66, SEQ ID NO: 74, or SEQ ID NO: 75 including post-translational modifications of that sequence (SEQ ID NO: 7, SEQ ID NO: 8, SEQ ID NO: 17, SEQ ID NO: 18, SEQ ID NO: 27, SEQ ID NO: 28, SEQ ID NO: 35, SEQ ID NO: 36, SEQ ID NO: 45, SEQ ID NO: 46, SEQ ID NO: 55, SEQ ID NO: 56, SEQ ID NO: 65, SEQ ID NO: 66, SEQ ID NO: 74, or SEQ ID NO: 75).

In certain embodiments, the extracellular antigen-binding domain of a presently disclosed antigen-recognizing receptor (e.g., CAR) cross-competes for binding to DLL3 (e.g., human DLL3) with a reference antibody or an antigen-binding fragment thereof comprising the V_{H} CDR1, CDR2, and CDR3 sequences and the V_{L} CDR1, CDR2, and CDR3 sequences of, for example, any one of the presently disclosed scFvs (e.g.,). In certain embodiments, the extracellular antigen-binding domain of a presently disclosed antigen-recognizing receptor (e.g., CAR) cross-competes for binding to DLL3 (e.g., human DLL3) with a reference antibody or an antigen-binding portion thereof comprising the V_{H} and V_{L} sequences of, for example, any one of the presently disclosed scFvs (e.g., SC16.8, SC16.13, SC16.15, SC16.25, SC16.67, SC16.118, SC16.125, and SC16.126).

In certain embodiments, the extracellular antigen-binding domain of a presently disclosed antigen-recognizing receptor (e.g., CAR) cross-competes for binding to DLL3 (e.g., human DLL3) with a reference antibody or an antigen-binding portion thereof comprising the V_{H} CDR1, CDR2, and CDR3 sequences and the V_{L} CDR1, CDR2, and CDR3 sequences of scFv SC16.8. For example, the extracellular antigen-binding domain of a presently disclosed antigen-recognizing receptor (e.g., CAR) cross-competes for binding to DLL3 (e.g., human DLL3) with a reference antibody or an antigen-binding portion thereof comprising a V_{H} comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a V_{L} comprising a CDR1 comprising the amino acid sequence set forth in SEQ ID NO: 4, a CDR2 comprising amino acids having the sequence set forth in SEQ ID NO: 5, and a CDR3 comprising amino acids having the sequence set forth in SEQ ID NO: 6. In certain embodiments, the extracellular antigen-binding domain of a presently disclosed antigen-recognizing receptor (e.g., CAR) cross-competes for binding to DLL3 (e.g., human DLL3) with a reference antibody or an antigen-binding portion thereof comprising the V_{H} and V_{L} sequences of scFv SC16.8. For example, the extracellular antigen-binding domain of a presently disclosed antigen-recognizing receptor (e.g., CAR) cross-competes for binding to DLL3 (e.g., human DLL3) with a reference antibody or an antigen-binding portion thereof comprising a V_{H} comprising amino acids having the sequence set forth in SEQ ID NO: 7, and a V_{L} comprising amino acids having the sequence set forth in SEQ ID NO: 8.

In certain embodiments, the extracellular antigen-binding domain binds to the same epitope region on DLL3 (e.g., human DLL3) as the reference antibody or antigen-binding portion thereof. For example, the extracellular antigen-binding domain of a presently disclosed CAR binds to the same epitope region on DLL3 (e.g., human DLL3) as a reference antibody or an antigen-binding portion thereof comprising the V_{H} CDR1, CDR2, and CDR3 sequences and the V_{L} CDR1, CDR2, and CDR3 sequences of, for example, any one of the presently disclosed scFvs (e.g., SC16.8, SC16.13, SC16.15, SC16.25, SC16.67, SC16.118, SC16.125, and SC16.126). In certain embodiments, the extracellular antigen-binding domain of a presently disclosed CAR binds to the same epitope region on DLL3 (e.g., human DLL3) as a reference antibody or an antigen-binding portion thereof comprising the V_{H} and V_{L} sequences of, for example, any one of the presently disclosed scFvs (e.g., SC16.8, SC16.13, SC16.15, SC16.25, SC16.67, SC16.118, SC16.125, and SC16.126).

In certain embodiments, the extracellular antigen-binding domain cross-competes for binding to DLL3 (e.g., human DLL3) with a reference antibody or an antigen-binding portion thereof. For example, the extracellular antigen-binding domain of a presently disclosed CAR cross-competes for binding to DLL3 (e.g., human DLL3) as a reference antibody or an antigen-binding portion thereof comprising the V_{H} CDR1, CDR2, and CDR3 sequences and the V_{L} CDR1, CDR2, and CDR3 sequences of, for example, any one of the presently disclosed scFvs (e.g., SC16.8, SC16.13, SC16.15, SC16.25, SC16.67, SC16.118, SC16.125, and SC16.126). In certain embodiments, the extracellular antigen-binding domain of a presently disclosed CAR binds to the same epitope region on DLL3 (e.g., human DLL3) as a reference antibody or an antigen-binding portion thereof comprising the V_{H} and V_{L} sequences of, for example, any one of the presently disclosed scFvs (e.g., SC16.8, SC16.13, SC16.15, SC16.25, SC16.67, SC16.118, SC16.125, and SC16.126).

Extracellular antigen-binding domains that cross-compete or compete with the reference antibody or antigen-binding portions thereof for binding to DLL3 (e.g., human DLL3) can be identified by using routine methods known in the art, including, but not limited to, ELISAs, radioimmunoassays (RIAs), Biacore, flow cytometry, Western blotting, and any other suitable quantitative or qualitative antibody-binding assays. Competition ELISA is described in Morris, “Epitope Mapping of Protein Antigens by Competition ELISA”, The Protein Protocols Handbook (1996), pp 595-600, edited by J. Walker, which is incorporated by reference in its entirety. In certain embodiments, the antibody-binding assay comprises measuring an initial binding of a reference antibody to a DLL3 polypeptide, admixing the reference antibody with a test extracellular antigen-binding domain, measuring a second binding of the reference antibody to the DLL3 polypeptide in the presence of the test extracellular antigen-binding domain, and comparing the initial binding with the second binding of the reference antibody, wherein a decreased second binding of the reference antibody to the DLL3 polypeptide in comparison to the initial binding indicates that the test extracellular antigen-binding domain cross-competes with the reference antibody for binding to DLL3, e.g., one that recognizes the same or substantially the same epitope, an overlapping epitope, or an adjacent epitope. In certain embodiments, the reference antibody is labeled, e.g., with a fluorochrome, biotin, or peroxidase. In certain embodiments, the DLL3 polypeptide is expressed in cells, e.g., in a flow cytometry test. In certain embodiments, the DLL3 polypeptide is immobilized onto a surface, including a Biacore ship (e.g., in a Biacore test), or other media suitable for surface plasmon resonance analysis. The binding of the reference antibody in the presence of a completely irrelevant antibody (that does not bind to DLL3) can serve as the control high value. The control low value can be obtained by incubating a labeled reference antibody with an unlabeled reference antibody, where competition and reduced binding of the labeled reference antibody would occur. In certain embodiments, a test extracellular antigen-binding domain that reduces the binding of the reference antibody to a DLL3 polypeptide by at least about 20%, at least about 30%, at least about 40%, at least about 50%, at least about 60%, at least about 70%, at least about 80%, at least about 90%, or at least about 95% is considered to be an extracellular antigen-binding domain that cross-competes with the reference antibody for binding to DLL3. In certain embodiments, the assays are performed at room temperature.

In certain embodiments, the antibody-binding assay comprises measuring an initial binding of a test extracellular antigen-binding domain to a DLL3 polypeptide, admixing the test extracellular antigen-binding domain with a reference antibody, measuring a second binding of the test extracellular antigen-binding domain to the DLL3 polypeptide in the presence of the reference antibody, and comparing the initial binding with the second binding of the test extracellular antigen-binding domain, where a decreased second binding of the test extracellular antigen-binding domain to the DLL3 polypeptide in comparison to the initial binding indicates that the test extracellular antigen-binding domain cross-competes with the reference antibody for binding to DLL3, e.g., one that recognizes the same or substantially the same epitope, an overlapping epitope, or an adjacent epitope. In certain embodiments, the test extracellular antigen-binding domain is labeled, e.g., with a fluorochrome, biotin, or peroxidase. In certain embodiments, the DLL3 polypeptide is expressed in cells, e.g., in a flow cytometry test. In certain embodiments, the DLL3 polypeptide is immobilized onto a surface, including a Biacore ship (e.g., in a Biacore test), or other media suitable for surface plasmon resonance analysis. The binding of the test extracellular antigen-binding domain in the presence of a completely irrelevant antibody (that does not bind to DLL3) can serve as the control high value. The control low value can be obtained by incubating a labeled test extracellular antigen-binding domain with an unlabeled test extracellular antigen-binding domain, where competition and reduced binding of the labeled test extracellular antigen-binding domain would occur. In certain embodiments, a test extracellular antigen-binding domain, whose binding to a DLL3 polypeptide is decreased by at least about 20%, at least about 30%, at least about 40%, at least about 50%, at least about 60%, at least about 70%, at least about 80%, at least about 90%, or at least about 95% in the presence of a reference antibody, is considered to be an extracellular antigen-binding domain that cross-competes with the reference antibody for binding to DLL3. In certain embodiments, the assays are performed at room temperature.

In certain non-limiting embodiments, the extracellular antigen-binding domain of the presently disclosed CAR comprises a linker connecting the heavy chain variable region and light chain variable region of the extracellular antigen-binding domain. In certain embodiments, the linker comprises or consists of the amino acid sequence set forth in SEQ ID NO: 78. In certain embodiments, the linker comprises or consists of the amino acid sequence set forth in SEQ ID NO: 79. In certain embodiments, the linker comprises or consists of the amino acid sequence set forth in SEQ ID NO: 80. In certain embodiments, the linker comprises or consists of the amino acid sequence set forth in SEQ ID NO: 81. In certain embodiments, the linker comprises or consists of the amino acid sequence set forth in SEQ ID NO: 82. In certain embodiments, the linker comprises or consists of the amino acid sequence set forth in SEQ ID NO: 83.

In certain embodiments, the variable regions within the extracellular antigen-binding domain have to be linked one after another such that at the N-terminus of the extracellular antigen-binding domain, a heavy chain variable region (V_{H}) is positioned. In certain embodiments, if the extracellular antigen-binding domain is an scFv, the variable regions are positioned from the N- to the C-terminus: V_{H}-V_{L}.

In certain embodiments, the variable regions within the extracellular antigen-binding domain have to be linked one after another such that at the N-terminus of the extracellular antigen-binding domain, a light chain variable region (V_{L}) is positioned. In certain embodiments, if the extracellular antigen-binding domain is an scFv, the variable regions are positioned from the N- to the C-terminus: V_{L}-V_{H}.

### 3.2. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR.

In certain embodiments, the CAR comprises an extracellular antigen-binding domain that specifically binds to DLL3, a transmembrane domain, and an intracellular signaling domain.

### 3.2.1. Extracellular Antigen-Binding Domain of a CAR

The extracellular antigen-binding domain of the CAR can be any extracellular antigen-binding domain disclosed herein, e.g., in Section 3.1. In certain embodiments, the CAR comprises an extracellular antigen-binding domain disclosed in Section 3.1.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus of the extracellular antigen-binding domain. In certain embodiments, the signal peptide comprises a CD8 polypeptide, e.g., the CAR comprises a truncated CD8 signal peptide.

### 3.2.2. Transmembrane Domain of a CAR

In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of CD8 or a fragment thereof, a native or modified transmembrane domain of CD28 or a fragment thereof, a native or modified transmembrane domain of CD3ζ or a fragment thereof, a native or modified transmembrane domain of CD4 or a fragment thereof, a native or modified transmembrane domain of 4-1BB or a fragment thereof, a native or modified transmembrane domain of OX40 or a fragment thereof, a native or modified transmembrane domain of ICOS or a fragment thereof, a native or modified transmembrane domain of CD84 or a fragment thereof, a native or modified transmembrane domain of CD166 or a fragment thereof, a native or modified transmembrane domain of CD8a or a fragment thereof, a native or modified transmembrane domain of CD8b or a fragment thereof, a native or modified transmembrane domain of ICAM-1 or a fragment thereof, a native or modified transmembrane domain of CTLA-4 or a fragment thereof, a native or modified transmembrane domain of CD27 or a fragment thereof, a native or modified transmembrane domain of CD40 or a fragment thereof, NKGD2 or a fragment thereof, or a combination thereof.

In certain embodiments, the transmembrane domain of the CAR comprises a CD8 polypeptide (e.g., a transmembrane domain of CD8 or a fragment thereof). In certain embodiments, the transmembrane domain of the CAR comprises a transmembrane domain of human CD8 or a fragment thereof. In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_001139345.1 (SEQ ID NO: 85) or a fragments thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 85, which is at least 20, or at least 30, or at least 40, or at least 50, and up to 235 amino acids in length. Alternatively or additionally, in non-limiting various embodiments, the CD8 polypeptide comprises or consists of an amino acid sequence of amino acids 1 to 235, 1 to 50, 50 to 100, 100 to 150, 150 to 200, 137 to 209 or 200 to 235 of SEQ ID NO: 85. In certain embodiments, the transmembrane domain of the CAR comprises a CD8 polypeptide comprising or consisting of amino acids 137 to 209 of SEQ ID NO: 85. SEQ ID NO: 85 is provided below.

[SEQ ID NO: 85]
MALPVTALLLPLALLLHAARPSQFRVSPLDRTWNLGETVELKCQV
LLSNPTSGCSWLFQPRGAAASPTFLLYLSQNKPKAAEGLDTQRFS
GKRLGDTFVLTLSDFRRENEGYYFCSALSNSIMYFSHFVPVFLPA
KPTTTPAPRPPTPAPTIASQPLSLRPEACRPAAGGAVHTRGLDFA
CDIYIWAPLAGTCGVLLLSLVITLYCNHRNRRRVCKCPRPVVKSG
DKPSLSARYV

In certain embodiments, the transmembrane domain of the CAR comprises a transmembrane domain of mouse CD8 or a fragment thereof. In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: AAA92533.1 (SEQ ID NO: 86) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 86, which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or consists of an amino acid sequence of amino acids 1 to 247, 1 to 50, 50 to 100, 100 to 150, 150 to 200, 151 to 219, or 200 to 247 of SEQ ID NO: 86. In certain embodiments, the transmembrane domain of the CAR comprises a CD8 polypeptide comprising or consisting of amino acids 151 to 219 of SEQ ID NO: 86. SEQ ID NO: 86 is provided below.

[SEQ ID NO: 86]
MASPLTRFLSLNLLLMGESIILGSGEAKPQAPELRIFPKKMDAEL
GQKVDLVCEVLGSVSQGCSWLFQNSSSKLPQPTFVVYMASSHNKI
TWDEKLNSSKLFSAVRDTNNKYVLTLNKFSKENEGYYFCSVISNS
VMYFSSVVPVLQKVNSTTTKPVLRTPSPVHPTGTSQPQRPEDCRP
RGSVKGTGLDFACDIYIWAPLAGICVAPLLSLIITLICYHRSRKR
VCKCPRPLVRQEGKPRPSEKIV

In certain embodiments, the transmembrane domain of a presently disclosed CAR comprises a CD28 polypeptide (e.g., a transmembrane domain of CD28 or a fragment thereof).

In certain embodiments, the transmembrane domain of the CAR comprises a transmembrane domain of human CD28 or a fragment thereof. In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_006130 (SEQ ID No: 87) or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 87 which is at least 20, or at least 30, or at least 40, or at least 50, and up to 220 amino acids in length. Alternatively or additionally, in non-limiting various embodiments, the CD28 polypeptide comprises or consists of an amino acid sequence of amino acids 1 to 220, 1 to 50, 50 to 100, 100 to 150, 150 to 200, 153 to 179, or 200 to 220 of SEQ ID NO: 87. In certain embodiments, the transmembrane domain of the CAR comprises a CD28 polypeptide comprising or consisting of amino acids 153 to 179 of SEQ ID NO: 87. SEQ ID NO: 87 is provided below:

[SEQ ID NO: 87]
MLRLLLALNLFPSIQVTGNKILVKQSPMLVAYDNAVNLSCKYSYN
LFSREFRASLHKGLDSAVEVCVVYGNYSQQLQVYSKTGFNCDGKL
GNESVTFYLQNLYVNQTDIYFCKIEVMYPPPYLDNEKSNGTIIHV
KGKHLCPSPLFPGPSKPFWVLVVVGGVLACYSLLVTVAFIIFWVR
SKRSRLLHSDYMNMTPRRPGPTRKHYQPYAPPRDFAAYRS

In certain embodiments, the transmembrane domain of the CAR comprises a CD28 polypeptide (e.g., a transmembrane domain of mouse CD28 or a fragment thereof). In certain embodiments, the CD28 polypeptide comprises or consists of an amino acid sequence that is at least about 85%, about 90%, about 95%, about 96%, about 97%, about 98%, about 99% or 100% homologous or identical to the amino acid sequence having a NCBI Reference No: NP_031668.3 (SEQ ID No: 88) or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 88, which is at least 20, or at least 30, or at least 40, or at least 50, and up to 218 amino acids in length. Alternatively or additionally, in non-limiting various embodiments, the CD28 polypeptide comprises or consists of an amino acid sequence of amino acids 1 to 220, 1 to 50, 50 to 100, 100 to 150, 150 to 200, 151 to 177, or 200 to 218 of SEQ ID NO: 88. In certain embodiments, the transmembrane domain of the CAR comprises a CD28 polypeptide comprising or consisting of amino acids 151 to 177 of SEQ ID NO: 88. SEQ ID NO: 88 is provided below:

[SEQ ID NO: 88]
MTLRLLFLALNFFSVQVTENKILVKQSPLLVVDSNEVSLSCRYSY
NLLAKEFRASLYKGVNSDVEVCVGNGNFTYQPQFRSNAEFNCDGD
FDNETVTFRLWNLHVNHTDIYFCKIEFMYPPPYLDNERSNGTIIH
IKEKHLCHTQSSPKLFWALVVVAGVLFCYGLLVTVALCVIWTNSR
RNRLLQSDYMNMTPRRPGLTRKPYQPYAPARDFAAYRP

In certain non-limiting embodiments, the CAR further comprises a spacer region that links the extracellular antigen-binding domain to the transmembrane domain. The spacer region can be flexible enough to allow the antigen binding domain to orient in different directions to facilitate antigen recognition while preserving the activating activity of the CAR.

In certain embodiments, the hinge/spacer region of the CAR comprises a native or modified hinge region of CD8 or a fragment thereof, a native or modified hinge region of CD28 or a fragment thereof, a native or modified hinge region of CD3ζ or a fragment thereof, a native or modified hinge region of CD40 or a fragment thereof, a native or modified hinge region of 4-1BB or a fragment thereof, a native or modified hinge region of OX40 or a fragment thereof, a native or modified hinge region of CD84 or a fragment thereof, a native or modified hinge region of CD166 or a fragment thereof, a native or modified hinge region of CD8a or a fragment thereof, a native or modified hinge region of CD8b or a fragment thereof, a native or modified hinge region of ICOS or a fragment thereof, a native or modified hinge region of ICAM-1 or a fragment thereof, a native or modified hinge region of CTLA-4 or a fragment thereof, a native or modified hinge region of CD27 or a fragment thereof, a native or modified hinge region of CD40 or a fragment thereof, a native or modified hinge region of NKGD2 or a fragment thereof, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 87 or 88), a portion of a CD8 polypeptide (e.g., a portion of SEQ ID NO: 85 or 86), a variation of any of the foregoing which is at least about 80%, at least about 85%, at least about 90%, at least about 95%, or at least about 100% homologous or identical thereto, or a synthetic spacer sequence.

### 3.2.3. Intracellular Signaling Domain of a CAR

In certain embodiments, the CAR comprises an intracellular signaling domain. In certain non-limiting embodiments, the intracellular signaling domain of the CAR comprises a CD3ζ polypeptide. CD3ζ can activate or stimulate a cell (e.g., a cell of the lymphoid lineage, e.g., a T cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T cell) after antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the CD3ζ polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_932170 (SEQ ID NO: 89) or a fragment thereof, and/or may optionally comprise up to one or up to two or up to three conservative amino acid substitutions. In certain non-limiting embodiments, the CD3ζ polypeptide comprises or consists of an amino acid sequence that is a consecutive portion of SEQ ID NO: 89, which is at least 20, or at least 30, or at least 40, or at least 50, and up to 164 amino acids in length. Alternatively or additionally, in non-limiting various embodiments, the CD3ζ polypeptide comprises or consists of an amino acid sequence of amino acids 1 to 164, 1 to 50, 50 to 100, 52 to 164, 100 to 150, or 150 to 164 of SEQ ID NO: 89. In certain embodiments, the intracellular signaling domain of the CAR comprises a CD3ζ polypeptide comprising or consisting of amino acids 52 to 164 of SEQ ID NO: 89. SEQ ID NO: 89 is provided below:

[SEQ ID NO: 89]
MKWKALFTAAILQAQLPITEAQSFGLLDPKLCYLLDGILFIYGVI
LTALFLRVKFSRSADAPAYQQGQNQLYNELNLGRREEYDVLDKRR
GRDPEMGGKPQRRKNPQEGLYNELQKDKMAEAYSEIGMKGERRRG
KGHDGLYQGLSTATKDTYDALHMQALPPR

In certain embodiments, the intracellular signaling domain of the CAR comprises a CD3ζ polypeptide comprising or consisting of the amino acid sequence set forth in SEQ ID NO: 90. SEQ ID NO: 90 is provided below.

[SEQ ID NO: 90]
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR

An exemplary nucleotide sequence encoding the amino acid sequence of SEQ ID NO: 90 is set forth in SEQ ID NO: 91, which is as provided below.

[SEQ ID NO: 91]
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGAGAAGGAAGAACCCTCAGGAAGGCCTGTACAAT
GAACTGCAGAAAGATAAGATGGCGGAGGCCTACAGTGAGATTGGG
ATGAAAGGCGAGCGCCGGAGGGGCAAGGGGCACGATGGCCTTTAC
CAGGGTCTCAGTACAGCCACCAAGGACACCTACGACGCCCTTCAC
ATGCAGGCCCTGCCCCCTCGC

In certain embodiments, the intracellular signaling domain of the CAR further comprises at least a co-stimulatory signaling region. In certain embodiments, the co-stimulatory signaling region comprises at least one co-stimulatory molecule or a fragment thereof. In certain embodiments, the co-stimulatory signaling region comprises an intracellular domain of at least one co-stimulatory molecule or a fragment thereof.

As used herein, a “co-stimulatory molecule” refers to a cell surface molecule other than antigen receptor or its ligand that can provide an efficient response of lymphocytes to an antigen. In certain embodiments, a co-stimulatory molecule can provide optimal lymphocyte activation. Non-limiting examples of co-stimulatory molecules include CD28, 4-1BB, OX40, ICOS, DAP-10, CD27, CD40, NKGD2, CD2, FN14, HVEM, LTBR, CD28H, TNFR1, TNFR2, BAFF-R, BCMA, TACI, TROY, RANK, CD40, CD27, CD30, EDAR, XEDAR, GITR, DR6, and NGFR, and combinations thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen-recognizing receptor (e.g., a chimeric antigen receptor (CAR)) binds to its target antigen. As one example, a 4-1BB ligand (i.e., 4-1BBL) may bind to 4-1BB for providing an intracellular signal that in combination with a CAR signal induces an effector cell function of the CAR⁺ T cell.

In certain embodiments, the intracellular signaling domain of the CAR comprises a co-stimulatory signaling region that comprises a CD28 polypeptide, e.g., an intracellular domain of CD28 or a fragment thereof. In certain embodiments, the intracellular signaling domain of the CAR comprises a co-stimulatory signaling region that comprises a CD28 polypeptide, e.g., an intracellular domain of human CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 87 or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 87, which is at least 20, or at least 30, or at least 40, or at least 50, and up to 220 amino acids in length. Alternatively or additionally, in non-limiting various embodiments, the CD28 polypeptide comprises or consists of an amino acid sequence of amino acids 1 to 220, 1 to 50, 50 to 100, 100 to 150, 114 to 220, 150 to 200, 180 to 220, or 200 to 220 of SEQ ID NO: 87. In certain embodiments, the intracellular signaling domain of the CAR comprises a co-stimulatory signaling region that comprises a CD28 polypeptide comprising or consisting of an amino acid sequence of amino acids 180 to 220 of SEQ ID NO: 87.

In certain embodiments, the intracellular signaling domain of the CAR comprises a co-stimulatory signaling region that comprises a CD28 polypeptide, e.g., an intracellular domain of mouse CD28 or a fragment thereof. In certain embodiments, the CD28 polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 88 or a fragment thereof, and/or may optionally comprise up to one or up to two or up to three conservative amino acid substitutions. In non-limiting certain embodiments, the CD28 polypeptide comprises or consists of an amino acid sequence that is a consecutive portion of SEQ ID NO: 88, which is at least about 20, or at least about 30, or at least about 40, or at least about 50, and up to 218 amino acids in length. Alternatively or additionally, in non-limiting various embodiments, the CD28 polypeptide comprises or consists of an amino acid sequence of amino acids 1 to 218, 1 to 50, 50 to 100, 100 to 150, 150 to 218, 178 to 218, or 200 to 218 of SEQ ID NO: 88. In certain embodiments, the co-stimulatory signaling region of a presently disclosed CAR comprises a CD28 polypeptide that comprises or consists of the amino acids 178 to 218 of SEQ ID NO: 88.

In certain embodiments, the co-stimulatory signaling region of a presently disclosed CAR comprises a CD28 polypeptide comprising a mutated YMNM motif. CD28 is a transmembrane protein that plays a critical role in T cell activation through its function as a costimulatory molecule. CD28 possesses an intracellular domain, which comprises intracellular motifs that are critical for the effective signaling of CD28. In certain embodiments, the CD28 intracellular domain comprises intracellular subdomains (also known as “intracellular motifs”) that regulate signaling pathways post TCR-stimulation. CD28 includes three intracellular motifs: a YNM motif, and two proline-rick motifs: PRRP motif, and PYAP motif. The CD28 intracellular motifs can serve as docking sites for a number of adaptor molecules that interact with these motifs through their SH2 or SH3 domains. Such interaction transduces downstream signals terminating on transcription factors that regulate gene expression. For example, a native YMNM motif binds to a p85 subunit of a phosphoinositide 3-kinase (PI3K). A native YMNM motif also binds to growth factor receptor-bound protein 2 (Grb2) and/or Grb2-related adaptor protein 2 (GADS). Grb2 binds to Gab1 and Gab2, which in turn can recruit the p85 subunit of a PI3K.

In certain embodiments, a native YMNIV motif consists of the amino acid sequence set forth in YMNM (SEQ ID NO: 92). In certain embodiments, a native YMNM motif binds to the p85 subunit of PI3K via a consensus sequence YMxM (SEQ ID NO: 93), wherein x is not an asparagine (N). In certain embodiments, a native YMNM motif binds to Grb2 and/or GADs via a consensus sequence YxNx (SEQ ID NO: 94), wherein x is not a methionine (M).

In certain embodiments, the CD28 polypeptide comprising a presently disclosed mutated YMNM motif has reduced recruitment of the p85 subunit of a PI3K as compared to a CD28 molecule comprising a native YMNM motif. In certain embodiments, the p85 subunit of a PI3K does not bind to the mutated YMNM motif, thereby reducing the recruitment of the p85 subunit of a PI3K to the CD28 polypeptide. The mutated YMNM motif that blocks the binding of the p85 subunit of a PI3K retains its binding to Grb2 and/or GADS. Thus, downstream signaling of Grb2/GADS remains intact, e.g., downstream signaling leading to IL-2 secretion remains intact. Such mutated YNM motif is referred to as “GADS/Grb2-permitting mutant”.

In certain embodiments, the mutated YMNM binds to the p85 subunit of a PI3K, but does not bind to Grb2 and/or GADS. Since the binding of PI3K p85 is retained, the downstream signaling of PI3K retains intact. Since the binding of Grb2/GADS is blocked, the recruitment of PI3K p85 subunit, which is triggered by the binding of Grb2 to Gab1 and Gab2, is reduced or blocked. In addition, the downstream signaling of Grb2/GADS is blocked. Such mutated YMNM motif is referred to as “PI3K-permissive mutant”.

In certain embodiments, the mutated YMNM does not bind to the p85 subunit of a PI3K, and does not bind to Grb2 and/or GADS. Such mutated YMNM motif is referred to as “non-functional mutant”. Non-functional mutants do not provide binding of PI3K, Grb2, or GADS to CD28 at the YMNM motif, but do not preclude these signaling molecules from binding elsewhere in the CD28 molecule.

In certain embodiments, the mutated YMNM retains only one methionine residue of the two methionine residues present in the YMNM motif, i.e. YMxx or YxxM. These motifs potentially modulate signaling via PI3K by limiting how many methionine residues can bind the p85 subunit of PI3K. Such mutated YMNM motif is referred to as “hybrid ‘HEMI’ mutant”.

In certain embodiments, the mutated YMNM motif is a GADS/Grb-2 permitting mutant. In certain embodiments, the mutated YMNM motif consists of the amino acid sequence set forth in YxNx (SEQ ID NO: 94), wherein x is not a methionine (M). In certain embodiments, x is selected from the group consisting of amino acids A, R, N, D, C, E, Q, G, H, I, K, F, P, S, T, W, Y, V, and L. In certain embodiments, the mutated YMNM motif consists of the amino acid sequence set forth in YENV (SEQ ID NO: 95), YSNV (SEQ ID NO: 96), YKNL (SEQ ID NO: 97), YENQ (SEQ ID NO: 98), YKNI (SEQ ID NO: 99), YINQ (SEQ ID NO: 100), YHNK (SEQ ID NO: 101), YVNQ (SEQ ID NO: 102), YLNP (SEQ ID NO: 103), YLNT (SEQ ID NO: 104), YDND (SEQ ID NO: 105), YENI (SEQ ID NO: 106), YENL (SEQ ID NO: 107), YKNQ (SEQ ID NO: 108), YKNV (SEQ ID NO: 109), or YANG (SEQ ID NO: 110). In certain embodiments, the mutated YMNM motif consists of the amino acid sequence set forth in YSNV (SEQ ID NO: 96). In certain embodiments, the mutated YMNM motif consists of the amino acid sequence set forth in YKNI (SEQ ID NO: 99). In certain embodiments, the mutated YMNM motif consists of the amino acid sequence set forth in YENV (SEQ ID NO: 95). In certain embodiments, the mutated YMNM motif consists of the amino acid sequence set forth in YKNL (SEQ ID NO: 97).

In certain embodiments, the mutated YMNM motif is a PI3K-permissive mutant. In certain embodiments, the mutated YMNM motif consists of the amino acid sequence set forth in YMxM (SEQ ID NO: 93), wherein x is not an asparagine (N). In certain embodiments, x is selected from the group consisting of amino acids A, R, D, C, E, Q, G, H, I, K, M, F, P, S, T, W, Y, V, and L. In certain embodiments, the mutated YMNM motif consists of the amino acid sequence set forth in YMDM (SEQ ID NO: 111), YMPM (SEQ ID NO: 112), YMRM (SEQ ID NO: 113), or YMSM (SEQ ID NO: 114). In certain embodiments, the mutated YMNM motif consists of the amino acid sequence set forth in YMDM (SEQ ID NO: 111).

In certain embodiments, the mutated YMNM motif consists of the amino acid sequence set forth in YbxM (SEQ ID NO: 115), wherein x is not an asparagine (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMTN4 motif consists of the amino acid sequence set forth in YTHM (SEQ ID NO: 116), YVLM (SEQ ID NO: 117), YIAM (SEQ ID NO: 118), YVEM (SEQ ID NO: 119), YVKM (SEQ ID NO: 120), or YVPM (SEQ ID NO: 121).

In certain embodiments, the mutated YMNM motif consists of the amino acid sequence set forth in YMxb (SEQ ID NO: 122), wherein x is not an asparagine (N), and b is not a methionine (M). In certain embodiments, x is selected from the group consisting of amino acids A, R, D, C, E, Q, G, H, I, K, M, F, P, S, T, W, Y, V, and L. In certain embodiments, b is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMAP (SEQ ID NO: 123).

Certain mutated YMNM motifs are described in Mol Cell Proteomics. 2010 November; 9(11):2391-404; Virology. 2015 May; 0: 568-577, both of which are incorporated by reference herein in its entirety.

In certain embodiments, the mutated YMNM motif is a hybrid ‘HEMI’ mutant. In certain embodiments, the mutated YMNM motif consists of the amino acid sequence set forth in YMNx (SEQ ID NO: 124) or YxNM (SEQ ID NO: 125), wherein x is not a methionine (M). In certain embodiments, x is selected from the group consisting of amino acids A, R, N, C, E, Q, G, H, I, K, N, F, P, S, T, W, Y, V, and L. In certain embodiments, the mutated YMNM motif consists of the amino acid sequence set forth in YMNV (SEQ ID NO: 126), YENM (SEQ ID NO: 127), YMNQ (SEQ ID NO: 128), YMNL (SEQ ID NO: 129), or YSNM (SEQ ID NO: 130).

In certain embodiments, the mutated YMNM motif is a non-functional mutant. In certain embodiments, the mutated YMNM motif consists of the amino acid sequence Ybxb (SEQ ID NO: 131), wherein x is not an asparagine (N), and b is not a methionine (M). In certain embodiments, x is selected from the group consisting of A, R, D, C, E, Q, G, H, I, K, M, F, P, S, T, W, Y, V, and L. In certain embodiments, b is selected from the group consisting of A, R, N, D, C, E, Q, G, H, I, K, F, P, S, T, W, Y, V, and L. In certain embodiments, the mutated YMNM motif consists of the amino acid sequence set forth in YGGG (SEQ ID NO: 132), YAAA (SEQ ID NO: 133), YFFF (SEQ ID NO: 134), YETV (SEQ ID NO: 135), YQQQ (SEQ ID NO: 136), YHAE (SEQ ID NO: 137), YLDL (SEQ ID NO: 138), YLIP (SEQ ID NO: 139), YLRV (SEQ ID NO: 140), YTAV (SEQ ID NO: 141), or YVHV (SEQ ID NO: 142). In certain embodiments, the mutated YMNM motif consists of the amino acid sequence set forth in YGGG (SEQ ID NO: 132).

In certain embodiments, the intracellular signaling domain of the presently disclosed chimeric receptor comprises a co-stimulatory signaling domain that comprises a CD28 polypeptide comprising a mutated YNM motif consisting of the amino acid sequence set forth in YENV (SEQ ID NO: 95), wherein the CD28 polypeptide comprises or consists of the amino acid sequence set forth in SEQ ID NO: 143. SEQ ID NO: 143 is provided below.

[SEQ ID NO: 143]
RSKRSRLLHSDYENVTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YKNI (SEQ ID NO: 99), wherein the CD28 polypeptide comprises or consists of the amino acid sequence set forth in SEQ ID NO: 144. SEQ ID NO: 144 is provided below.

[SEQ ID NO: 144]
RSKRSRLLHSDYKNITPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YNM motif consisting of the amino acid sequence set forth in YMDM (SEQ ID NO: 111), wherein the CD28 polypeptide comprises or consists of the amino acid sequence set forth in SEQ ID NO: 145. SEQ ID NO: 145 is provided below.

[SEQ ID NO: 145]
RSKRSRLLHSDYMDMTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YGGG (SEQ ID NO: 132), wherein the CD28 polypeptide comprises or consists of the amino acid sequence set forth in SEQ ID NO: 146. SEQ ID NO: 146 is provided below.

[SEQ ID NO: 146]
RSKRSRLLHSDYGGGTPRRPGPTRKHYQPYAPPRDFAAYRS

In certain embodiments, the intracellular signaling domain of the presently disclosed chimeric receptor comprises a co-stimulatory signaling domain that comprises a CD28 polypeptide comprising a mutated YMNM motif consisting of the amino acid sequence set forth in YSNV (SEQ ID NO: 96), wherein the CD28 polypeptide comprises or consists of the amino acid sequence set forth in SEQ ID NO: 147. SEQ ID NO: 147 is provided below.

[SEQ ID NO: 147]
RSKRSRLLHSDYSNVTPRRPGPTRKHYQPYAPPRDFAAYRS

In certain embodiments, the intracellular signaling domain of the presently disclosed CAR comprises a first co-stimulatory signaling domain that comprises a CD28 polypeptide comprising a mutated YMNM motif (as disclosed herein), and a second co-stimulatory signaling domain that comprises an intracellular domain of a co-stimulatory molecule. Additional information regarding CARs including CD28 polypeptide comprising a mutated YMNM motif can be found in International Patent Publication No. WO 2021/158850, which is incorporated by reference in its entirety.

In certain embodiments, the intracellular signaling domain of the CAR comprises a co-stimulatory signaling region that comprises a 4-1BB polypeptide, e.g., an intracellular domain of 4-1BB or a fragment thereof. In certain embodiments, the intracellular signaling domain of the CAR comprises a co-stimulatory signaling region that comprises a 4-1BB polypeptide, e.g., an intracellular domain of human 4-1BB or a fragment thereof. In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Ref. No.: NP_001552 (SEQ ID NO: 148) or a fragment thereof, and/or may optionally comprise up to one or up to two or up to three conservative amino acid substitutions. In non-limiting certain embodiments, the 4-1BB polypeptide comprises or consists of an amino acid sequence that is a consecutive portion of SEQ ID NO: 148, which is at least 20, or at least 30, or at least 40, or at least 50, or at least 100, or at least 150, or at least 150, and up to 255 amino acids in length. Alternatively or additionally, in non-limiting various embodiments, the 4-1BB polypeptide comprises or consists of an amino acid sequence of amino acids 1 to 255, 1 to 50, 50 to 100, 100 to 150, 150 to 200, or 200 to 255 of SEQ ID NO: 148. In certain embodiments, the intracellular signaling domain of the CAR comprises a co-stimulatory signaling region that comprises a 4-1BB polypeptide comprising or consisting of an amino acid sequence of amino acids 214 to 255 of SEQ ID NO: 148. SEQ ID NO: 148 is provided below.

[SEQ ID NO: 148]
MGNSCYNIVATLLLVLNFERTRSLQDPCSNCPAGTFCDNNRNQIC
SPCPPNSFSSAGGQRTCDICRQCKGVFRTRKECSSTSNAECDCTP
GFHCLGAGCSMCEQDCKQGQELTKKGCKDCCFGTFNDQKRGICRP
WTNCSLDGKSVLVNGTKERDVVCGPSPADLSPGASSVTPPAPARE
PGHSPQIISFFLALTSTALLFLLFFLTLRFSVVKRGRKKLLYIFK
QPFMRPVQTTQEEDGCSCRFPEEEEGGCEL

In certain embodiments, the intracellular signaling domain of the CAR comprises a co-stimulatory signaling region that comprises intracellular domains of two or more co-stimulatory molecules or portions thereof, e.g., an intracellular domain of CD28 or a fragment thereof and an intracellular domain of 4-1BB or a fragment thereof, or an intracellular domain of CD28 or a fragment thereof and an intracellular domain of OX40 or a fragment thereof.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

### 3.2.4. Exemplified CARs

In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 87. In certain embodiments, the intracellular signaling domain comprises (i) a CD3ζ polypeptide comprising the amino acid sequence set forth in SEQ ID NO: 90, and (ii) a co-stimulatory signaling region comprising a CD28 polypeptide comprising amino acids 180 to 220 of SEQ ID NO: 87. In certain embodiments, the V_{H} and V_{L} are linked via a linker comprising or consisting of the amino acid sequence set forth in SEQ ID NO: 79. In certain embodiments, the V_{H} and V_{L} are positioned from the N- to the C-terminus: V_{L}-V_{H}. In certain embodiments, the CAR is designed as “SC16.8_h28z”.

In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide comprising a mutated YMNM motif consisting of the amino acid sequence set forth in YSNV (SEQ ID NO: 96). In certain embodiments, the transmembrane domain comprises a CD28 polypeptide that comprises amino acids 153 to 179 of SEQ ID NO: 87. In certain embodiments, the intracellular signaling domain comprises (i) a CD3ζ polypeptide comprising the amino acid sequence set forth in SEQ ID NO: 90, and (ii) a co-stimulatory signaling region comprising a CD28 polypeptide comprises or consists of the amino acid sequence set forth in SEQ ID NO: 147.

In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a 4-1BB polypeptide (e.g., an intracellular domain of human 4-1BB or a fragment thereof). In certain embodiments, the transmembrane domain comprises a CD28 polypeptide that comprises amino acids 153 to 179 of SEQ ID NO: 87. In certain embodiments, the intracellular signaling domain comprises (i) a CD3ζ polypeptide comprising the amino acid sequence set forth in SEQ ID NO: 90, and (ii) a co-stimulatory signaling region comprising a 4-1BB polypeptide comprising amino acids 214 to 255 of SEQ ID NO: 148. In certain embodiments, the V_{H} and V_{L} are linked via a linker comprising or consisting of the amino acid sequence set forth in SEQ ID NO: 79. In certain embodiments, the V_{H} and V_{L} are positioned from the N- to the C-terminus: V_{L}-V_{H}. In certain embodiments, the CAR is designed as “SC16.8_hBBz”.

### 3.3. TCR Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

### 4. Cells

The presently disclosed subject matter provides cells comprising a presently disclosed DLL3-targeted antigen-recognizing receptor (e.g., one disclosed in Section 4.3). In certain embodiments, the cell is selected from the group consisting of cells of lymphoid lineage, cells of myeloid lineage, stem cells from which cells of lymphoid lineage can be derived, and stem cells from which cells of myeloid lineage can be derived. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and R heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

In certain embodiments, the cells can be transduced with the presently disclosed DLL3-targeted antigen-recognizing receptor such that the cells express the antigen-recognizing receptor.

In certain embodiments, the presently disclosed cell further comprises a soluble single-chain variable fragment (scFv) that binds a polypeptide that has immunosuppressive activity or immunostimulatory activity. In certain embodiments, immunosuppressive activity refers to induction of signal transduction or changes in protein expression in a cell (e.g., an activated immunoresponsive cell) resulting in a decrease in an immune response. Polypeptides known to suppress or decrease an immune response via their binding include CD47, PD-1, CTLA-4, and their corresponding ligands, including SIRPα, PD-L1, PD-L2, B7-1, and B7-2. Such polypeptides are present in the tumor microenvironment and inhibit immune responses to neoplastic cells. In various embodiments, inhibiting, blocking, or antagonizing the interaction of immunosuppressive polypeptides and/or their ligands enhances the immune response of the immunoresponsive cell.

In certain embodiments, the immunostimulatory activity refers to induction of signal transduction or changes in protein expression in a cell (e.g., an activated immunoresponsive cell) resulting in an increase in an immune response. Immunostimulatory activity may include pro-inflammatory activity. Polypeptides known to stimulate or increase an immune response via their binding include CD28, OX-40, 4-IBB, and their corresponding ligands, including B7-1, B7-2, OX-40L, and 4-1BBL. Such polypeptides are present in the tumor microenvironment and activate immune responses to neoplastic cells. In various embodiments, promoting, stimulating, or agonizing pro-inflammatory polypeptides and/or their ligands enhances the immune response of the immunoresponsive cell.

Cells comprising a CAR and a soluble scFv that binds a polypeptide that has immunosuppressive activity or immunostimulatory activity are disclosed in International Patent Publication No. WO 2014/134165, which is incorporated by reference in its entirety.

In certain embodiments, the presently disclosed cell further comprises an exogenous CD40L. Cells comprising a CAR and an exogenous CD40L are disclosed in International Patent Publication No. WO 2014/134165.

Additionally or alternatively, the presently disclosed cell is engineered to express IL-33. In certain embodiments, the presently disclosed cell further comprises an exogenous IL-33 polypeptide or a fragment thereof. In certain embodiments, the presently disclosed cell further comprises a modified promoter/enhancer at an IL-33 gene locus, which can increase IL-33 gene expression, e.g., a constitutive or inducible promoter placed to drive IL-33 gene expression. Cells comprising a chimeric receptor and engineered to express IL-33, e.g., comprising an exogenous IL-33 polypeptide or a fragment thereof or a modified promoter/enhancer at an IL-33 gene locus are disclosed in International Patent Publication No. WO2019/099479, which is incorporated by reference in its entirety.

Additionally or alternatively, the presently disclosed cell is engineered to express IL-36. In certain embodiments, the presently disclosed cell further comprises an exogenous IL-36 polypeptide or a fragment thereof. In certain embodiments, the presently disclosed cell further comprises a modified promoter/enhancer at an IL-36 gene locus, which can increase IL-36 gene expression, e.g., a constitutive or inducible promoter placed to drive IL-36 gene expression. Cells comprising a chimeric receptor and engineered to express IL-36, e.g., comprising an exogenous IL-36 polypeptide or a fragment thereof or a modified promoter/enhancer at an IL-36 gene locus are disclosed in International Patent Publication No. WO2019/099483, which is incorporated by reference in its entirety.

### 4.1. Interleukin-18 (IL-18)

In certain embodiments, the presently disclosed cell is engineered to express IL-18. In certain embodiments, the presently disclosed cell further comprises an exogenous IL-18 polypeptide or a fragment thereof. Interleukin 18 (also known as IGIF, IL-1g and IL1F4; GenBank ID: 3606 (human), 16173 (mouse), 29197 (rat), 403796 (dog), 100034216 (horse), 281249 (cattle)) is a gene encoding a proinflammatory cytokine that increases immune activity of certain immunoresponsive cells. The protein product of Interleukin 18 includes, but is not limited to, NCBI Reference Sequences NP_001553.1 and NP_001230140.1. IL-18 is produced by macrophages, T cells and other cells. IL-18 functions by binding to the interleukin-18 receptor, and together with other cytokines, such as IL-12, it can induce cell-mediated immunity, which following infection with microbial products. After stimulation with IL-18, natural killer (NK) cells and certain T cells release other cytokines, such as interferon-γ (IFNg), IL-2 and TNFa, which can further activate other types of immunoresponsive cells.

In certain embodiments, the term “IL-18” or “IL-18 cytokine” refers to the bioactive form of IL-18 after secretion from a cell (that is to say, where the signal peptide has been cleaved off). A non-limiting example of human IL-18 has the following amino acid sequence set forth in SEQ ID NO: 149, which is provided below.

[SEQ ID NO: 149]
YFGKLESKLSVIRNLNDQVLFIDQGNRPLFEDMTDSDCRDNAPRT
IFIISMYKDSQPRGMAVTISVKCEKISTLSCENKIISFKEMNPPD
NIKDTKSDIIFFQRSVPGHDNKMQFESSSYEGYFLACEKERDLFK
LILKKEDELGDRSIMFTVQNED

In certain embodiments, the exogenous IL-18 polypeptide is secretable. In certain embodiments, a secretable IL-18 polypeptide refers to a polypeptide or a protein, the cytokine portion of which has at least about 85%, about 90%, about 95%, about 96%, about 97%, about 98%, about 99%, or about 100% homologous to the cytokine portion of the protein product of IL-18 (GenBank ID: 3606 (human), 16173 (mouse), 29197 (rat), 403796 (dog), 100034216 (horse), 281249 (cattle)), or a fragment thereof that has immunostimulatory activity.

In certain embodiments, the secretable IL-18 polypeptide comprises a cytokine portion and a signal peptide, optionally joined by a linker peptide. Non-limiting examples of secretable IL-18 polypeptides include NCBI Reference Sequences NP_001553.1 and NP_001230140.1. An exemplary secretable IL-18 polypeptide is provided below.

[SEQ ID NO: 150]
MAAEPVEDNCINFVAMKFIDNTLYFIAEDDENLESDYFGKLESKL
SVIRNINDQVLFIDQGNRPLFEDMTDSDCRDNAPRTIFIISMYKD
SQPRGMAVTISVKCEKISTLSCENKIISFKEMNPPDNIKDTKSDI
IFFQRSVPGHDNKMQFESSSYEGYFLACEKERDLFKLILKKEDEL
GARSIMFTVQNED

In certain embodiments, the secretable IL-18 polypeptide comprises a signal sequence, for example, but not limited to the IL-2 signal sequence, the kappa leader sequence, the CD8 leader sequence or a peptide with essentially equivalent activity.

In certain embodiments, the IL-18 polypeptide is expressed from a vector. In certain embodiments, the IL-18 polypeptide comprises a heterologous signal sequence at the amino-terminus (that is to say, a signal sequence that is not naturally associated with IL-18). In certain embodiments, the heterologous signal sequence is selected from the group consisting of IL-2 signal sequence, the kappa leader sequence, the CD8 leader sequence, and combinations and/or synthetic variations thereof which retain the capacity to promote secretion of IL-18 polypeptide (either mature or non-mature)

In certain embodiments, the IL-18 polypeptide enhances an immune response of the immunoresponsive cell. In certain embodiments, the exogenous IL-18 polypeptide increases anti-tumor cytokine production. In certain embodiments, the anti-tumor cytokine is selected from the group consisting of IL-2, TNF-α and IFN-γ. In certain embodiments, the exogenous IL-18 polypeptide decreases the secretion of cytokines associated with cytokine release syndrome (CRS). In certain embodiments, the cytokines associated with cytokine release syndrome (CRS) is IL-6.

In certain embodiments, the presently disclosed cell further comprises a modified promoter/enhancer at an IL-18 gene locus, which can increase IL-18 gene expression, e.g., a constitutive or inducible promoter is placed to drive IL-18 gene expression. Cells comprising a chimeric receptor and engineered to express IL-18, e.g., comprising an exogenous IL-18 polypeptide or a fragment thereof or a modified promoter/enhancer at an IL-18 gene locus are disclosed in International Patent Publication No. WO2018/027155, which is incorporated by reference in its entirety.

### 4.2. Exemplified Cells

In certain embodiments, the cell comprises an antigen-recognizing receptor and an exogenous IL-18 polypeptide. In certain embodiments, the antigen-recognizing receptor is a CAR. In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide (e.g., an intracellular domain of human CD28 or a fragment thereof). In certain embodiments, the transmembrane domain comprises a CD28 polypeptide that comprises amino acids 153 to 179 of SEQ ID NO: 87. In certain embodiments, the intracellular signaling domain comprises (i) a CD3ζ polypeptide comprising the amino acid sequence set forth in SEQ ID NO: 90, and (ii) a co-stimulatory signaling region comprising a CD28 polypeptide comprising amino acids 180 to 220 of SEQ ID NO: 87. In certain embodiments, the exogenous IL-18 polypeptide is a human IL-18 polypeptide.

In certain embodiments, the cell comprises an antigen-recognizing receptor and an exogenous IL-18 polypeptide. In certain embodiments, the antigen-recognizing receptor is a CAR. In certain embodiments, the CAR is a DLL3-targeted CAR. In certain embodiments, the CAR comprises (a) an extracellular antigen-binding domain comprising (i) a V_{H} that comprises a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3, and (ii) a V_{L} that comprises a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; (b) a transmembrane domain comprising a CD28 polypeptide (e.g., a transmembrane domain of human CD28 or a fragment thereof), and (c) an intracellular signaling domain comprising (i) a CD3ζ polypeptide, and (ii) a co-stimulatory signaling region comprising a CD28 polypeptide comprising a mutated YMNM motif consisting of the amino acid sequence set forth in YSNV (SEQ ID NO: 96). In certain embodiments, the transmembrane domain comprises a CD28 polypeptide that comprises amino acids 153 to 179 of SEQ ID NO: 87. In certain embodiments, the intracellular signaling domain comprises (i) a CD3ζ polypeptide comprising the amino acid sequence set forth in SEQ ID NO: 90, and (ii) a co-stimulatory signaling region comprising a CD28 polypeptide comprises or consists of the amino acid sequence set forth in SEQ ID NO: 147. In certain embodiments, the exogenous IL-18 polypeptide is a human IL-18 polypeptide.

### 5. Nucleic Acid Compositions and Vectors

The present discloses subject matter provides a nucleic acid encoding a presently disclosed DLL3-targeted antigen-recognizing receptor (e.g., one disclosed in Section 3). Further provided are nucleic acid compositions comprising the nucleic acids disclosed herein. Also provided are cells comprising such nucleic acid compositions.

In certain embodiments, the nucleic acid composition further comprises a promoter that is operably linked to the presently disclosed DLL3-targeted antigen-recognizing receptor.

In certain embodiments, the promoter is endogenous or exogenous. In certain embodiments, the exogenous promoter is selected from an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiments, the promoter is an inducible promoter. In certain embodiment, the inducible promoter is selected from a NFAT transcriptional response element (TRE) promoter, a CD69 promoter, a CD25 promoter, and an IL-2 promoter.

The compositions and nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (e.g., gamma-retroviral vector or lentiviral vector) is employed for the introduction of the DNA construct into the cell. For example, a polynucleotide encoding an antigen-recognizing receptor can be cloned into a retroviral vector and expression can be driven from its endogenous promoter, from the retroviral long terminal repeat, or from a promoter specific for a target cell type of interest. Non-viral vectors may be used as well.

For initial genetic modification of a cell to include a presently disclosed DLL3-targeted antigen-recognizing receptor (e.g., a CAR), a retroviral vector can be employed for transduction, however any other suitable viral vector or non-viral delivery system can be used. The antigen-recognizing receptor can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-xB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller et al., (1985) Mol Cell Biol (1985); 5:431-437); PA317 (Miller., et al., Mol Cell Biol(1986); 6:2895-2902); and CRIP (Danos et al., Proc Natl Acad Sci USA (1988); 85:6460-6464). Non-amphotropic particles are suitable too, e.g., particles pseudotyped with VSVG, RD 114 or GALV envelope and any other known in the art.

Possible methods of transduction also include direct co-culture of the cells with producer cells (Bregni et al., Blood (1992); 80:1418-1422), or culturing with viral supernatant alone or concentrated vector stocks with or without appropriate growth factors and polycations (Xu et al., Exp Hemat (1994); 22:223-230; and Hughes et al. J Clin Invest (1992); 89:1817).

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Thera (1990); 15-14; Friedman, Science 244:1275-1281, 1989; Eglitis et al., BioTechniques (1988); 6:608-614; Tolstoshev et al., Cur Opin Biotechnol(1990); 1:55-61; Sharp, The Lancet (1991); 337:1277-78; Cornetta et al., Nucleic Acid Research and Molecular Biology 36:311-22, 1987; Anderson, Science (1984); 226:401-409; Moen, Blood Cells 17:407-16, 1991; Miller et al., Biotechnol (1989); 7:980-90; LeGal La Salle et al., Science (1993); 259:988-90; and Johnson, Chest (1995)107:77S-83S). Retroviral vectors are particularly well developed and have been used in clinical settings (Rosenberg et al., N Engl J Med (1990); 323:370, 1990; Anderson et al., U.S. Pat. No. 5,399,346).

Non-viral approaches can also be employed for genetic modification of a cell. For example, a nucleic acid molecule can be introduced into a cell by administering the nucleic acid in the presence of lipofection (Feigner et al., Proc Natl Acad Sci U.S.A. (1987); 84:7413; Ono et al., Neurosci Lett (1990); 17:259; Brigham et al., Am J Med Sci (1989); 298:278; Staubinger et al., Methods in Enzymol (1983); 101:512, Wu et al., J Biol Chem (1988); 263:14621; Wu et al., J Biol Chem (1989); 264:16985), or by micro-injection under surgical conditions (Wolff et al., Science (1990); 247:1465). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver a presently disclosed antigen-recognizing receptor to a cell or a subject. In certain embodiments, a CRISPR system is used to deliver a presently disclosed antigen-recognizing receptor disclosed herein. In certain embodiments, zinc-finger nucleases are used to deliver the antigen-recognizing receptor. In certain embodiments, a TALEN system is used to deliver a presently disclosed antigen-recognizing receptor.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10-9 to about 10-6 m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding an hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3″′-(((methylazanediyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9″′,9″″,9″″′-((((benzene-1,3,5-tricarbonyl)yris(azanediyl))tris(propane-3,1-diyl))tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4, 1-diyl))bis(azanetriyl))tetrakis(ethane-2,1-diyl) (9Z,9′Z,9″Z,9″′Z,12Z,12′Z,12″Z,12″′Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 6. Polypeptides

The presently disclosed subject matter provides methods for optimizing an amino acid sequence or a nucleic acid sequence by producing an alteration in the sequence. Such alterations may include certain mutations, deletions, insertions, or post-translational modifications. The presently disclosed subject matter further includes analogs of any naturally-occurring polypeptides disclosed herein (including, but not limited to, DLL3, CD8, CD28, 4-1BB, and CD3□). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous or identical to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the presently disclosed subject matter also provides fragments of any of the polypeptides disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

### 7. Formulations and Administration

The presently disclosed subject matter provides compositions comprising the presently disclosed cells. In certain embodiments, the compositions are pharmaceutical compositions further comprising a pharmaceutically acceptable carrier. Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Compositions comprising the presently disclosed cells can be provided systemically or directly to a subject for treating or ameliorating a disease or disorder. In certain embodiments, the presently disclosed cells or compositions comprising thereof are directly injected into an organ of interest (e.g., an organ affected by a neoplasia). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells (e.g., T cells or NK cells) in vitro or in vivo.

The presently disclosed cells can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 108 of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, 5×10⁵, 1×10⁶, about 5×10⁶, about 1×10⁷, about 5×10⁷, about 1×10⁸, or about 5×10⁸ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein and, the time for sequential administrations can be ascertained without undue experimentation.

In certain embodiments, the composition is a pharmaceutical composition comprising the presently disclosed cells and a pharmaceutically acceptable carrier.

Administration of the compositions can be autologous or heterologous. For example, cells can be obtained from one subject, and administered to the same subject or a different, compatible subject. Peripheral blood derived cells or their progeny (e.g., in vivo, ex vivo or in vitro derived) can be administered. When administering a presently disclosed composition (e.g., a pharmaceutical composition comprising presently disclosed cells), it can be formulated in a unit dosage injectable form (solution, suspension, emulsion).

The presently disclosed cells and compositions can be administered by any method known in the art including, but not limited to, oral administration, intravenous administration, subcutaneous administration, intranodal administration, intratumoral administration, intrathecal administration, intravitreal administration, intrapleural administration, intraosseous administration, intraperitoneal administration, pleural administration, and direct administration to the subject.

Additionally or alternatively, the presently disclosed subject matter also provides compositions comprising lipid nanoparticles (e.g., described in Section 5.5.1) including a nucleic acid or a nucleic acid composition disclosed herein. Compositions comprising the presently disclosed lipid nanoparticles can be conveniently provided as sterile and/or pyrogen-free. Compositions can be prepared to meet the standards of the United States Pharmacopoeia (USP), the European Pharmacopoeia (EP), the British Pharmacopoeia, and/or the International Pharmacopoeia.

Compositions including the presently disclosed lipid nanoparticles can include pharmaceutically acceptable excipients. Non-limiting examples of pharmaceutically acceptable excipients include inert diluents, dispersing agents, granulating agents, surface active agents, emulsifiers, disintegrating agents, binding agents, preservatives, buffering agents, lubricating agents, and/or oils. Furthermore, excipients such as cocoa butter and suppository waxes, coloring agents, coating agents, sweetening, flavoring, and/or perfuming agents can be present in the composition.

In certain embodiments, compositions including the presently disclosed lipid nanoparticles can be prepared as injectable preparations. These injectable preparations can include pharmaceutically acceptable vehicles and solvents including, without any limitation, water, Ringer's solution, U.S.P., isotonic sodium chloride solution, and/or oils (e.g., oleic acid). In certain embodiments, injectable preparations comprising the presently disclosed lipid nanoparticles can include a liquid suspension of crystalline or amorphous material with poor water solubility. Use of these poor water solubility materials allows to slow absorption from subcutaneous or intramuscular injection. Alternatively or additionally, compositions including the presently disclosed lipid nanoparticles can be prepared for rectal or vaginal administration, oral administration, topical and/or transdermal administration, intradermal administration, pulmonary administration, nasal administration, buccal administration, or ophthalmic administration. Additional information on various ways for formulating and preparing pharmaceutical compositions including the presently disclosed lipid nanoparticles can be found in Remington: The Science and Practice of Pharmacy, 22nd Edition, A. R. Gennaro, Lippincott, Williams & Wilkins, Baltimore, Md., 2012.

In certain embodiments, the compositions including the presently disclosed lipid nanoparticles can be formulated for controlled release or sustained release. As used herein, the term “controlled release” refers to a pharmaceutical composition or compound release profile that conforms to a particular pattern of release to effect a therapeutic outcome. As used herein, the term “sustained release” refers to a pharmaceutical composition or compound that conforms to a release rate over a specific period of time. The period of time may include, but is not limited to, hours, days, weeks, months and years.

Compositions comprising the presently disclosed lipid nanoparticles can be provided systemically or directly to a subject for inducing and/or enhancing an immune response to an antigen and/or treating and/or preventing a tumor, e.g., a tumor associated with MUC16. In certain embodiments, the presently disclosed lipid nanoparticles or compositions comprising thereof are provided in vivo to immunoresponsive cells. In certain embodiments, the presently disclosed lipid nanoparticles or compositions comprising thereof are directly injected into an organ of interest (e.g., an organ affected by a neoplasia). Alternatively, the presently disclosed lipid nanoparticles or compositions comprising thereof are provided indirectly to the organ of interest, for example, by administration into the circulatory system (e.g., the tumor vasculature). In certain embodiments, the presently disclosed lipid nanoparticles or compositions comprising thereof are provided ex vivo to immunoresponsive cells. Expansion and differentiation agents can be provided prior to, during or after administration of the lipid nanoparticles or compositions to increase production of cells (e.g., T cells or NK cells) ex vivo or in vivo.

The presently disclosed lipid nanoparticles can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 0.001 mg/kg to about 10 mg/kg, from about 0.005 mg/kg to about 10 mg/kg, from about 0.01 mg/kg to about 10 mg/kg, from about 0.05 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.05 mg/kg to about 5 mg/kg, from about 0.1 mg/kg to about 5 mg/kg, from about 1 mg/kg to about 5 mg/kg, from about 2 mg/kg to about 5 mg/kg, from about 0.0001 mg/kg to about 2.5 mg/kg, from about 0.001 mg/kg to about 2.5 mg/kg, from about 0.005 mg/kg to about 2.5 mg/kg, from about 0.01 mg/kg to about 2.5 mg/kg, from about 0.05 mg/kg to about 2.5 mg/kg, from about 0.1 mg/kg to about 2.5 mg/kg, from about 1 mg/kg to about 2.5 mg/kg, from about 2 mg/kg to about 2.5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, from about 0.05 mg/kg to about 1 mg/kg, from about 0.1 mg/kg to about 1 mg/kg, from about 0.0001 mg/kg to about 0.25 mg/kg, from about 0.001 mg/kg to about 0.25 mg/kg, from about 0.005 mg/kg to about 0.25 mg/kg, from about 0.01 mg/kg to about 0.25 mg/kg, from about 0.05 mg/kg to about 0.25 mg/kg, or from about 0.1 mg/kg to about 0.25 mg/kg of the presently disclosed lipid nanoparticles are administered to a subject.

In certain embodiments, between about 0.005 mg/kg to about 2.5 mg/kg, from about 0.1 mg/kg to about 1 mg/kg, or from about 0.05 mg/kg to about 1 mg/kg of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art. Dosages can be readily adjusted by those skilled in the art (e.g., a decrease in purity may require an increase in dosage).

### 8. Methods of Treatment

The presently disclosed cells and compositions comprising thereof can be used for treating or ameliorating a disease or disorder in a subject. In certain embodiments, the disease or disorder is associated with DLL3. In certain embodiments, the disease or disorder is associated with overexpression of DLL3.

In certain embodiments, the method comprises administering to a subject in need thereof the presently disclosed cells or compositions comprising thereof. In certain embodiments, the cell is a T cell. The T cell can be a CD4⁺ T cell or a CD8⁺ T cell. In certain embodiments, the T cell is a CD4⁺ T cell.

Additionally or alternatively, the presently disclosed lipid nanoparticles and compositions comprising thereof can be used for treating or ameliorating a disease or disorder in a subject. In certain embodiments, the disease or disorder is associated with DLL3. In certain embodiments, the disease or disorder is associated with overexpression of DLL3. In certain embodiments, the method comprises administering to a subject in need thereof the presently disclosed lipid nanoparticles or compositions comprising thereof.

For treatment, the amount administered is an amount effective in producing the desired effect. An effective amount can be provided in one or a series of administrations. An effective amount can be provided in a bolus or by continuous perfusion.

In certain embodiments, the disease or disorder is a tumor. In certain embodiments, the presently disclosed cells and compositions can reduce tumor burden, reduce the number of tumor cells, reduce tumor size, and/or eradicate the tumor in the subject, and/or increase or lengthen survival of the subject. In certain embodiments, the tumor is cancer.

Non-limiting examples of diseases and disorders include neuroendocrine tumors of the lung, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma. Non-limiting examples of neuroendocrine tumors of the lung include pulmonary neuroendocrine cancer (including typical carcinoid tumors, and atypical carcinoid tumors), large cell neuroendocrine carcinoma, and small-cell lung cancer. In certain embodiments, the tumor is small-cell lung cancer. In certain embodiments, the melanoma is uveal melanoma. In certain embodiments, the breast cancer is triple negative breast cancer.

Further modification can be introduced to the DLL3-specific CAR-expressing engineered immune cells (e.g., T cells) to avert or minimize the risks of immunological complications (known as “malignant T-cell transformation”), e.g., graft versus-host disease (GvHD). Modification of the engineered immune cells can include engineering a suicide gene into the DLL3-specific CAR-expressing T cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the C-terminus of the intracellular domain of the DLL3-specific CAR. The suicide gene can be included within the vector comprising nucleic acids encoding the presently disclosed DLL3-specific CARs.

The incorporation of a suicide gene into a presently disclosed DLL3-specific CAR gives an added level of safety with the ability to eliminate the majority of CAR T cells within a very short time period. A presently disclosed engineered immune cell (e.g., a T cell) incorporated with a suicide gene can be pre-emptively eliminated at a given time point post CAR T cell infusion, or eradicated at the earliest signs of toxicity.

### 9. Kits

The presently disclosed subject matter provides kits for or ameliorating a disease or disorder in a subject. In certain embodiments, the kit comprises the presently disclosed cells or a composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes a nucleic acid molecule encoding a presently disclosed DLL3-targeted antigen-recognizing receptor (e.g., a CAR).

If desired, the cells and/or nucleic acid molecules are provided together with instructions for administering the cells or nucleic acid molecules to a subject having or at risk of developing a disease or disorder. The instructions generally include information about the use of the composition for the treatment and/or prevention of a tumor or neoplasm. In certain embodiments, the instructions include at least one of the following: description of the therapeutic agent; dosage schedule and administration for treatment or prevention of a tumor or neoplasm; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the antibodies, multi-specific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### IL-18-Secreting CAR T Cells Targeting DLL3 are Highly Effective in Small Cell Lung Cancer Models

### Development of SC16-Based Single-Chain Variable Fragment

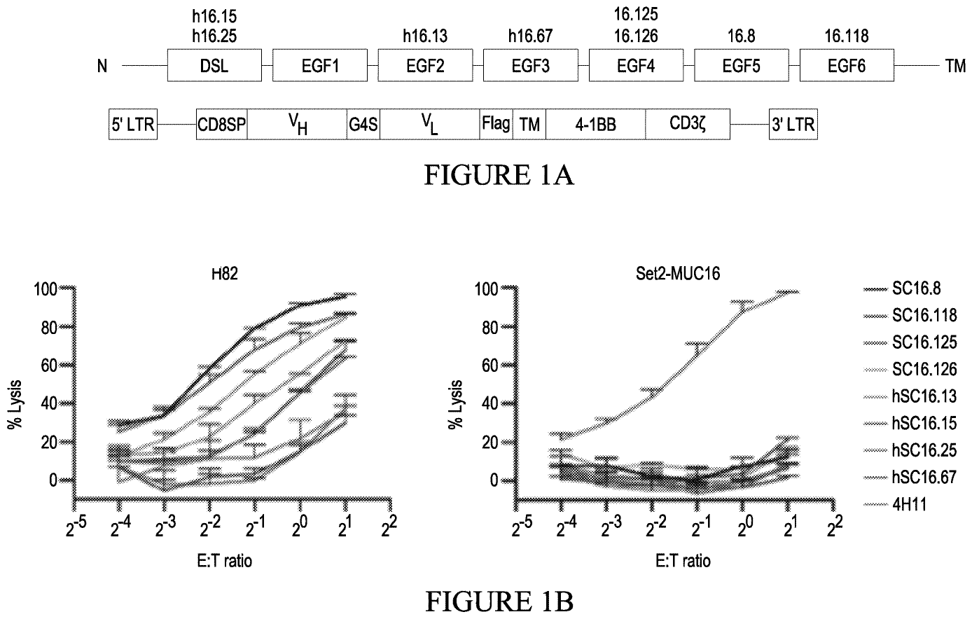

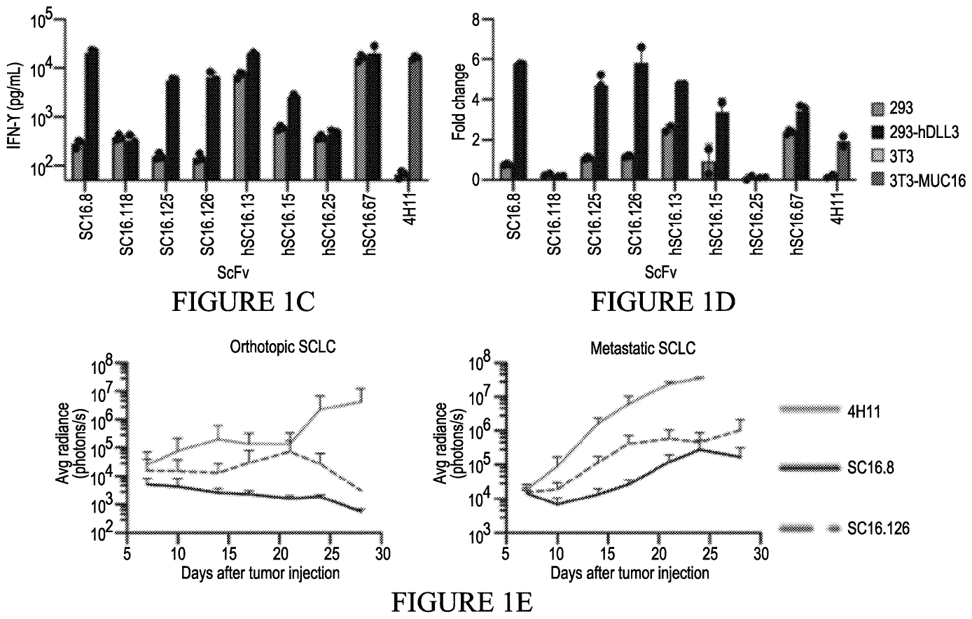

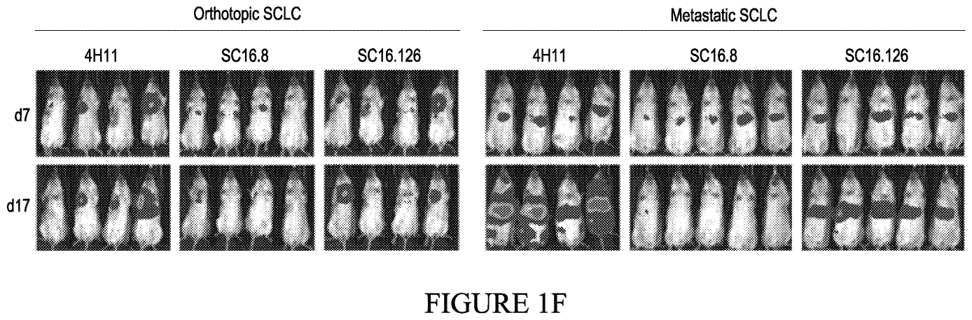

Previous work has identified a series of high-affinity monoclonal antibodies (SC16) with binding specificity to distinct extracellular domains of DLL3 (FIG. 1A). The presently disclosed subject matter initially sought to develop a highly functional CAR against DLL3 by cloning single-chain variable fragments (scFvs) derived from heavy- and light-chain pairs of these antibodies into a retroviral CAR vector with Flag-tag for easy detection (FIG. 1A). This vector contained the 4-1BB costimulation domain. The presently disclosed subject matter tested scFvs derived from SC16 monoclonal antibody clones 8, 13, 15, 25, 67, 118, 125, and 126, and isolated T cells from healthy donors were transduced with each CAR. The lysis potential of CAR T cells was measured with a 20-hour luciferase killing assay with DLL3+H82-SCLC cells. The 4H11 CAR, targeting the MUC16 protein, was used as negative control; Set2 cells (DLL3- and MUC16+) also served as controls for specificity. None of the DLL3 CARs showed lysis of Set2 cells. There was substantial variation in H82 lysis capacity of the SC16 CARs, with SC16.8, SC16.125, and SC16.126 performing the best (FIG. 1B).

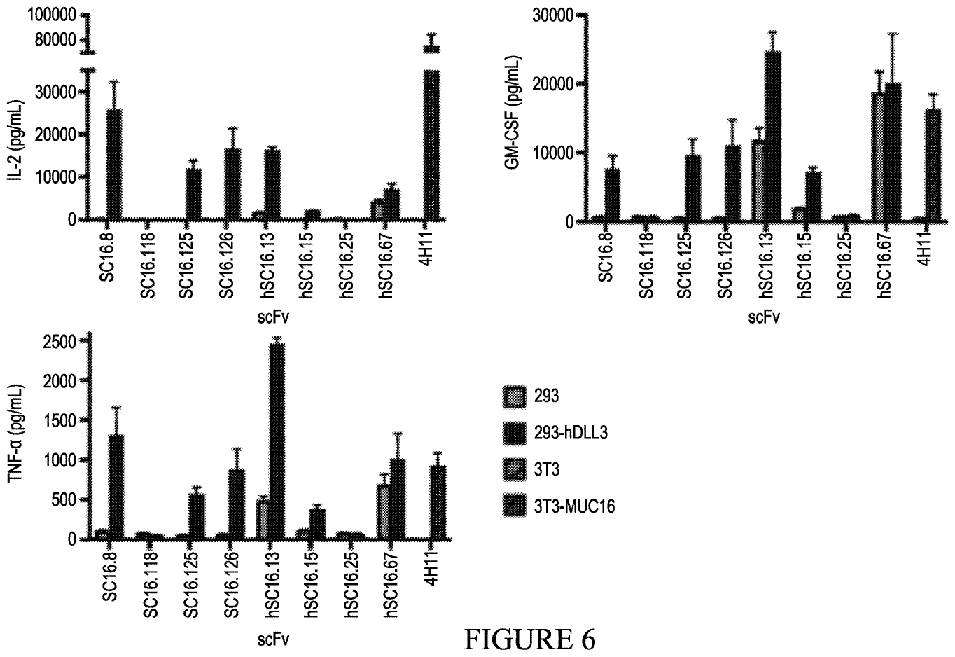

Next, coculture experiments were used to assess DLL3-mediated CAR T cell activation based on cytokine release and proliferative response. HEK293 cells (DLL3−) and HEK293 cells expressing human DLL3 were utilized for these experiments. The cytokines IFN-γ, IL-2, GM-CSF, and TNF-α were measured in the supernatant 24 hours after coculture, and CAR T cell proliferation was quantified after 7 days (FIGS. 1C, 1D, 6). DLL3-independent activation of humanized SC16.13 (hSC16.13) and hSC16.67 CAR T cells and minimal activation of SC16.118 and hSC16.25 CAR T cells was observed. Because the SC16.125 and SC16.126 CARs behaved similarly and both bind to the DLL3 EGF4 domain, SC16.8 and SC16.126 were chosen as lead candidates for in vivo testing. 1×10⁶ H82 cells were injected either in the lung parenchyma or tail vein of NSG mice. After 7 days mice were treated intravenously with 5×10⁶ CAR T cells. T cells expressing either the SC16.8 or SC16.126 CAR showed an antitumor response in both the orthotopic and metastatic SCLC model, compared with the negative control CAR 4H11 (FIG. 1, E and F). SC16.8 CAR T cells performed better than SC16.126 in both contexts. Thus, of the presently disclosed panel of eight SC16 clones, SC16.8 CAR T cells demonstrated the best DLL3-specific activation and antitumor efficacy.

### IL-18 Increases CAR T Cell Efficacy in a Murine Metastatic SCLC Model

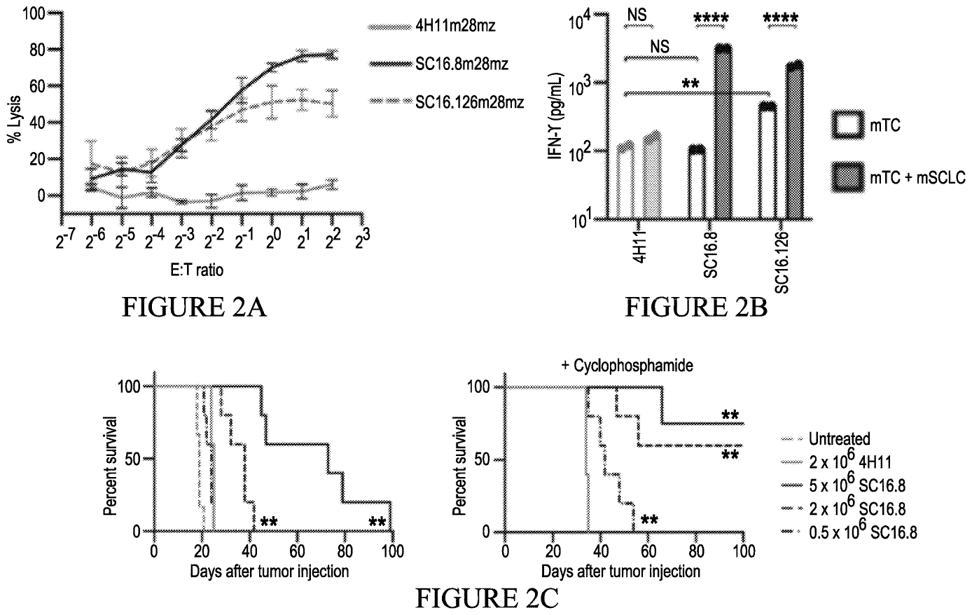

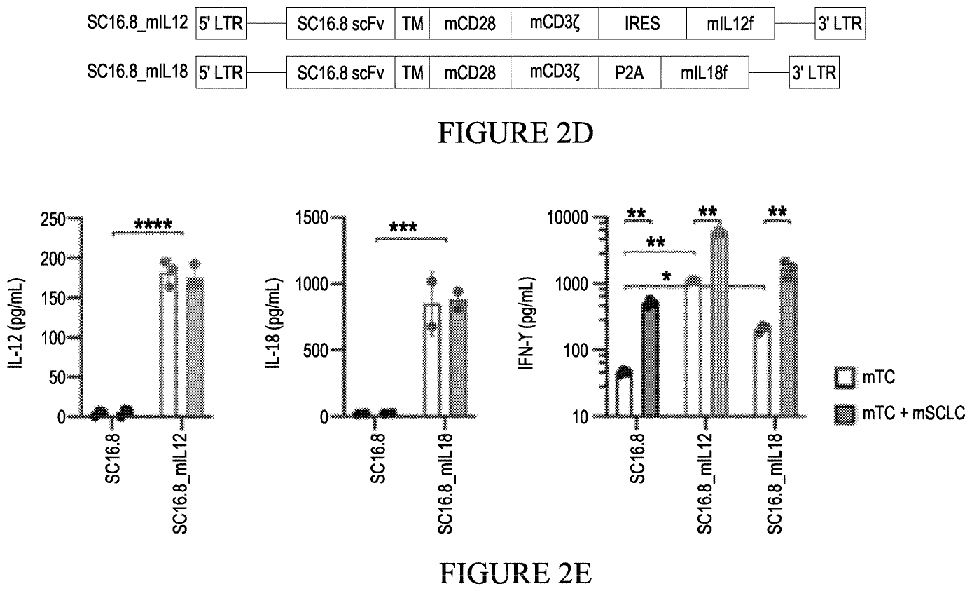

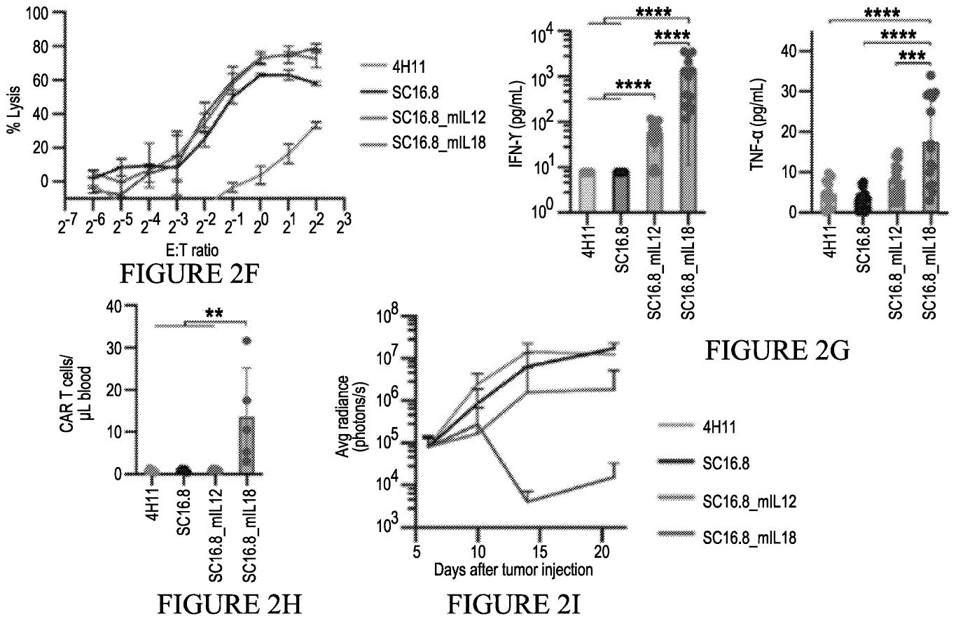

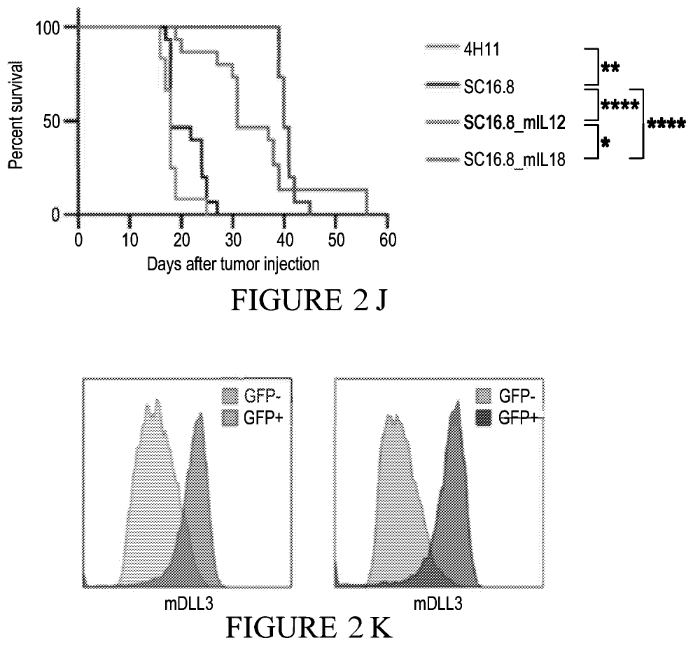

The presently disclosed subject matter next sought to explore the activity of these CAR T cells in an immunocompetent murine model, using a murine SCLC (mSCLC) cell line derived from a genetically engineered mouse model with concomitant loss of Rb1 and Trp53. First, it was tested whether the SC16.8 and SC16.126 CARs were cross-reactive to murine DLL3 by measuring the tumor lysis potential (FIG. 2A) and IFN-γ production (FIG. 2B) in coculture with mSCLC cells. Both CARs were activated by murine DLL3, but SC16.8 CAR T cells showed more lysis and higher target-specific IFN-γ levels. The presently disclosed subject matter proceeded with in vivo testing of SC16.8 CAR T cell functionality in a metastatic mSCLC model in immunocompetent mice based on tail vein injection of 1×10⁶ mSCLC allograft tumor cells. The amount of CAR T cells administered 7 days after systemic tumor cell injection with or without preconditioning chemotherapy of 50 mg/kg cyclophosphamide on day 6 was titrated (FIG. 2C). High doses of CAR T cells alone led to improved survival. Preconditioning chemotherapy induced long-term survival in several mice receiving 2×10⁶ or 5×10⁶ CAR T cells and improved survival of mice receiving a low dose of 0.5×10⁶ CAR T cells, compared with negative control 4H11 CAR T cells.

To investigate the impact of IL-12 or IL-18 secretion by the CAR T cells on antitumor efficacy, murine 1112 fusion or 1118 transgenes were cloned into a retroviral vector with SC16.8m28mz CAR (FIG. 2D). The secretion of murine IL-12 (mIL-12) and murine IL-18 (mIL-18) in the supernatant of murine CAR T cells alone or cocultured with mSCLC cells was validated (FIG. 2E). Functionality of the 2 cytokines was confirmed by the increase in IFN-γ in unstimulated CAR T cells, which further increased in the presence of mSCLC cells (FIG. 2E, right). As seen with other CARs, the lysis potential of all SC16.8 CAR T cell derivatives was similar, regardless of secreted cytokines (FIG. 2F).

Next, mice were treated with systemic mSCLC tumors with 2×10⁶ CAR T cells in the absence of preconditioning chemotherapy. Three days later whole blood and sera were collected for flow cytometry and cytokine analysis. A sharp increase in IFN-γ levels in the sera of mice treated with SC16.8 CAR T cells expressing mIL-12 (SC16.8_mIL12) or mIL-18 (SC16.8_mIL18) was observed, with the highest level when mIL-18 was secreted (FIG. 2G). IFN-γ levels in 4H11 and SC16.8 CAR T cell-treated mice were below the level of detection. TNF-α levels were increased in the sera of mice that received SC16.8_mIL18 CAR T cells (FIG. 2G). Circulating CAR T cells were detected only in the blood of mice treated with SC16.8_mIL18 CAR T cells (FIG. 2H). In addition, only mIL-18-secreting CAR T cells were able to shrink mSCLC tumors (FIG. 2I), which resulted in a prolonged survival of mice (FIG. 2J). Antigen loss was not detected, as surface expression of mDLL3 was still detected on end-stage tumors that progressed after treatment with SC16.8_mIL12 or SC16.8_mIL18 CAR T cells (FIG. 2K). In conclusion, mIL-18 secretion by CAR T cells improved the efficacy of DLL3-targeting CAR T cells in a murine metastatic SCLC model.

mIL-18 Stimulates CAR T Cell Proliferation and Activates Both CAR and Endogenous T Cells.

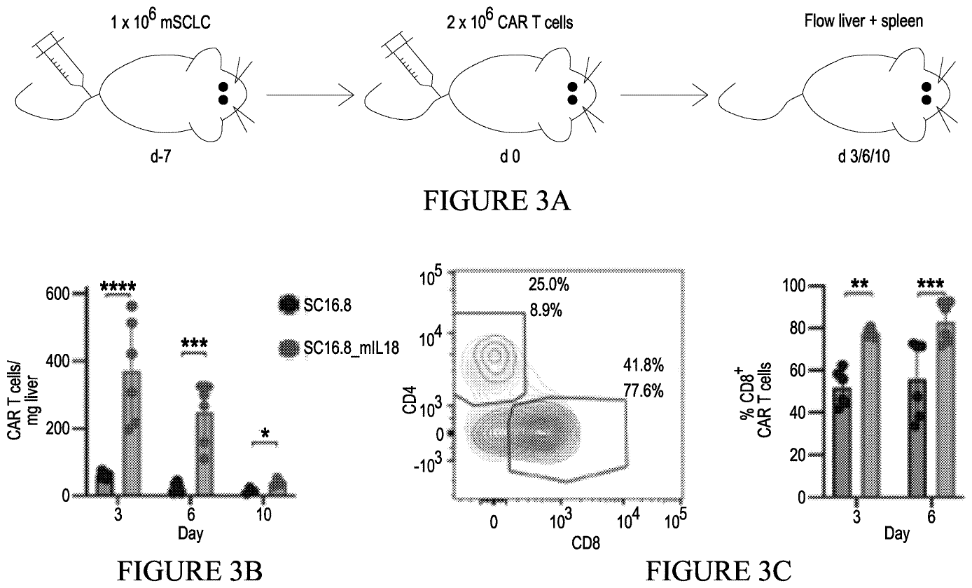

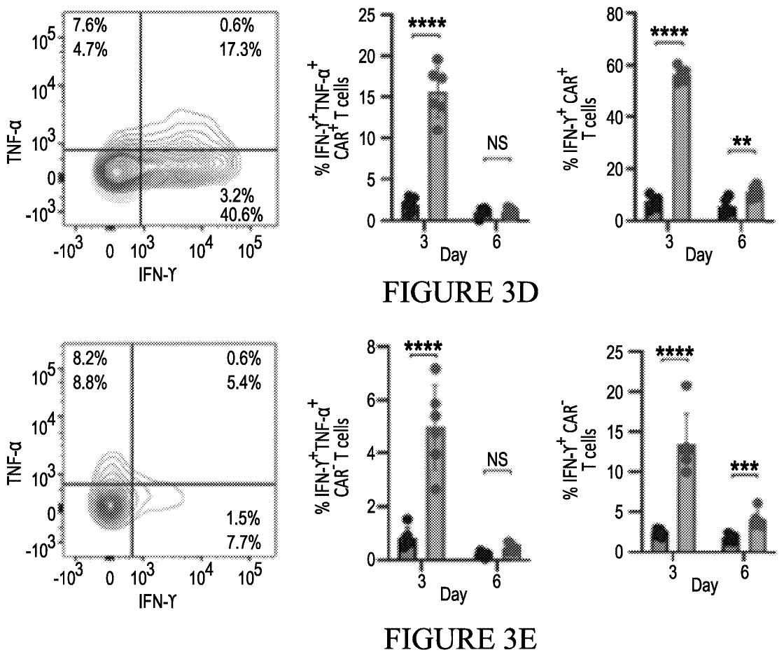

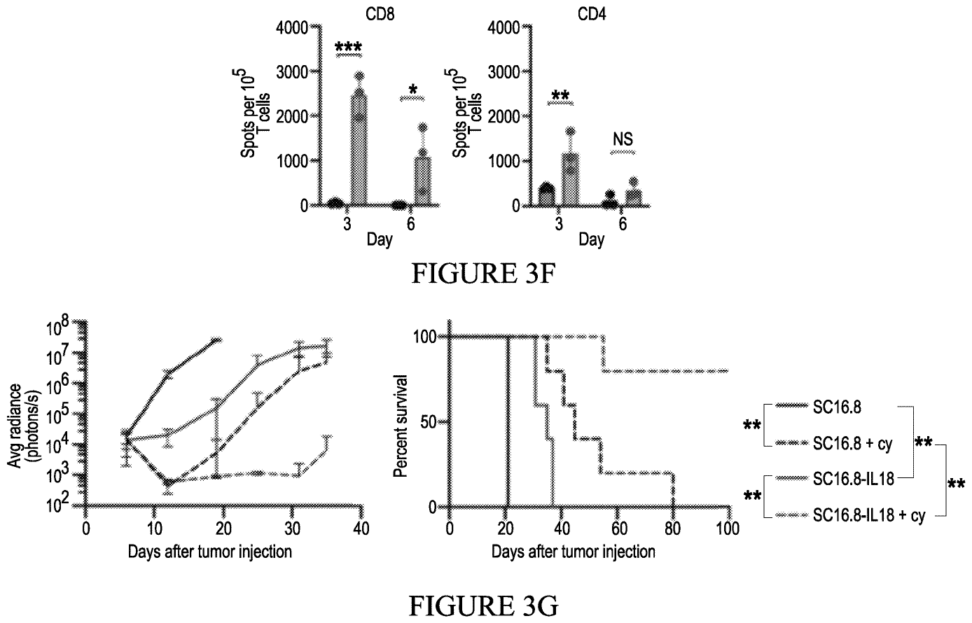

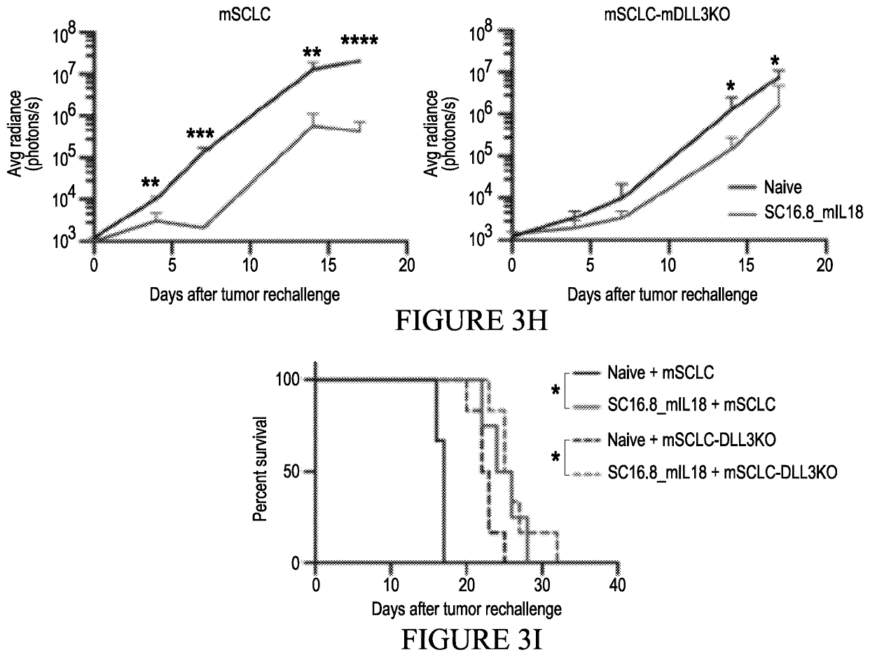

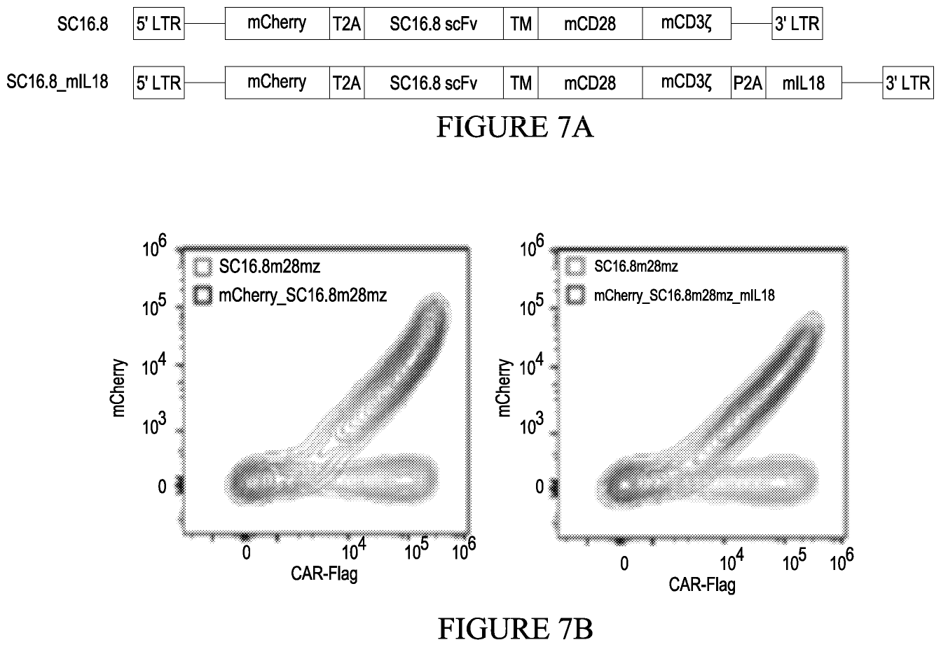

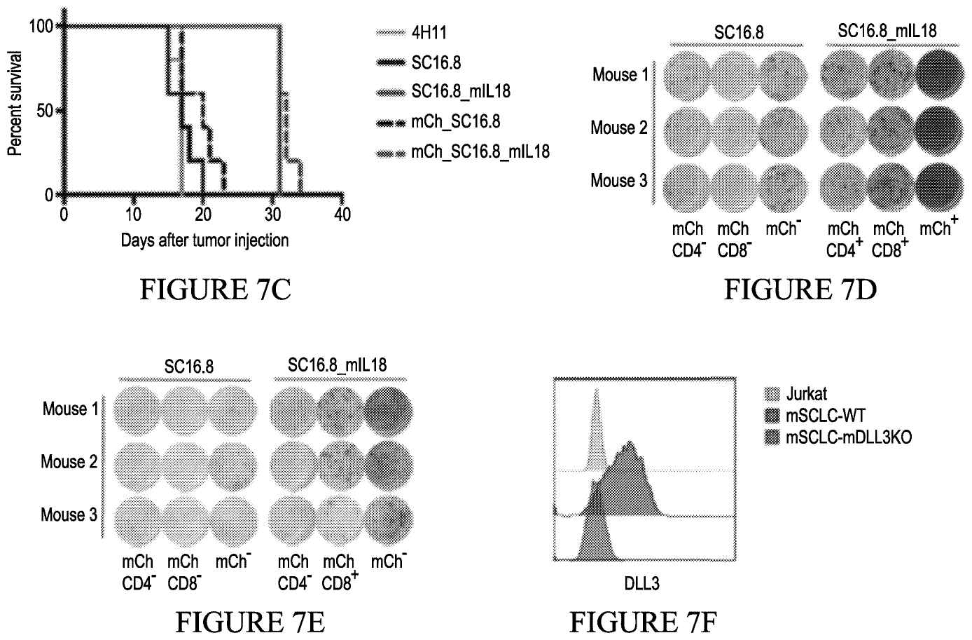

The presently disclosed subject matter studied the effect of mIL-18 on CAR T cells and TME by collecting livers (main tumor location) and spleens for flow cytometry analysis at various time points after CAR T cell treatment (FIG. 3A). In order to prevent potential undercounting because of CAR downregulation, mCherry was cloned into the retroviral vector for detection of transduced CAR T cells, and the antitumor efficacy was validated in mCherry+ CAR T cells in vivo (FIG. 7, A-C). mIL-18 induced more than 4-fold increase in the peak amount of CAR T cells in the liver, with a gradual reduction over time (FIG. 3B). T cells from days 3 and 6 were focused on for further analysis, because 10 days after treatment relatively few CAR T cells were detected. SC16.8_mIL18 CAR T cells in the liver were enriched for CD8+ (FIG. 3C). In addition to the increased number of SC16.8_mIL18 CAR T cells, increased activation was also observed, as quantified by intracellular staining for the effector cytokines IFN-γ and TNF-α (FIG. 3D). More IFN-γ+TNF-α+ double-positive cells were measured on day 3 when the CAR T cells secrete mIL-18 and an increased percentage of total IFN-γ+ CAR T cells on days 3 and 6. Intriguingly, a similar pattern of increased in IFN-γ+TNF-α+ and total IFN-γ+ cells was present in the CAR-bystander T cells (FIG. 3E). The presently disclosed subject matter then assessed whether the activated bystander T cells would demonstrate tumor reactivity. Endogenous mCherry− T cells were sorted from tumor-bearing livers 3 and 6 days after treatment with mCherry+ SC16.8 or SC16.8_mIL18 CAR T cells. Sorted CD4+ and CD8+ T cell populations were cocultured with mSCLC cells in an IFN-γ ELISpot assay (FIG. 3F). CAR T cell-secretion of mIL-18 increased recruitment of tumor-specific CD4+ (on day 3) and especially CD8+ (on days 3 and 6) endogenous T cells. Sorted SC16.8_mIL18 CAR T cells retained their ex vivo antitumor cell response to a greater degree and longer than SC16.8 CAR T cells (FIGS. 7D and 7E).

Because 2×10⁶ SC16.8_mIL18 CAR T cells did not lead to long-term survival in this aggressive mSCLC model when administered 7 days after tumor injection (FIG. 2J), it was tested whether cyclophosphamide pretreatment would sensitize the tumors to mIL-18-secreting CAR T cells. Indeed, a dramatic increase in antitumor response was observed when the mSCLC-bearing mice received cyclophosphamide 1 day prior to a low dose of 0.5×10⁶ CAR T cells. SC16.8_mIL18 CAR T cells were now able to induce long-term survival (FIG. 3G). Mice were rechallenged on day 42, to assess whether SC16.8_mIL18 CAR T cells are still present and active against tumor cells in mice that did not have detectable residual tumor after combination treatment of cyclophosphamide plus 0.5×10⁶ SC16.8_mIL18 CAR T cells. These mice were not fully protected against mSCLC outgrowth, but the tumor growth was delayed compared with tumor growth in naive age-matched control mice (FIG. 3H, left), suggestive of residual presence and activity of tumor-reactive T cells. It was also assessed whether the increased activation of tumor-reactive bystander T cells (FIGS. 3E and 3F) would lead to antitumor efficacy of these T cells independent of the CAR T cell response against mDLL3. Surviving mice with no evidence of tumor were rechallenged with mSCLC cells in which mDLL3 was knocked out (FIG. 7F). Again, the tumor outgrowth was slightly delayed (FIG. 3H, right), consistent with an endogenous T cell response against other mSCLC antigens. Survival of the rechallenged mice with either wild-type mSCLC or mSCLC-mDLL3KO was prolonged compared with the survival of naive mice (FIG. 3I). All together, these results demonstrate that the secretion of mIL-18 enhances the antitumor response of both CAR and endogenous T cells.

mIL-18 Reprograms and Activates the Myeloid Compartment of the TME.

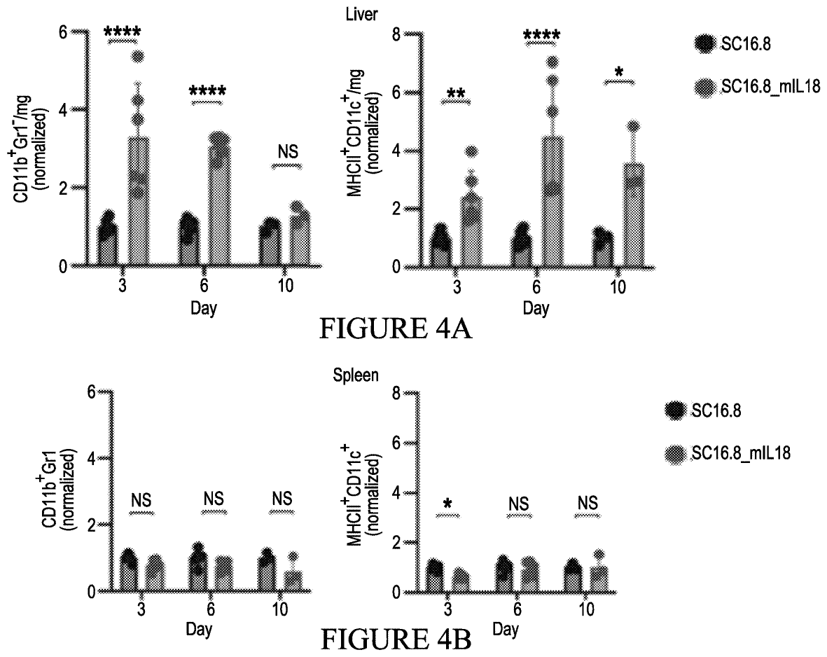

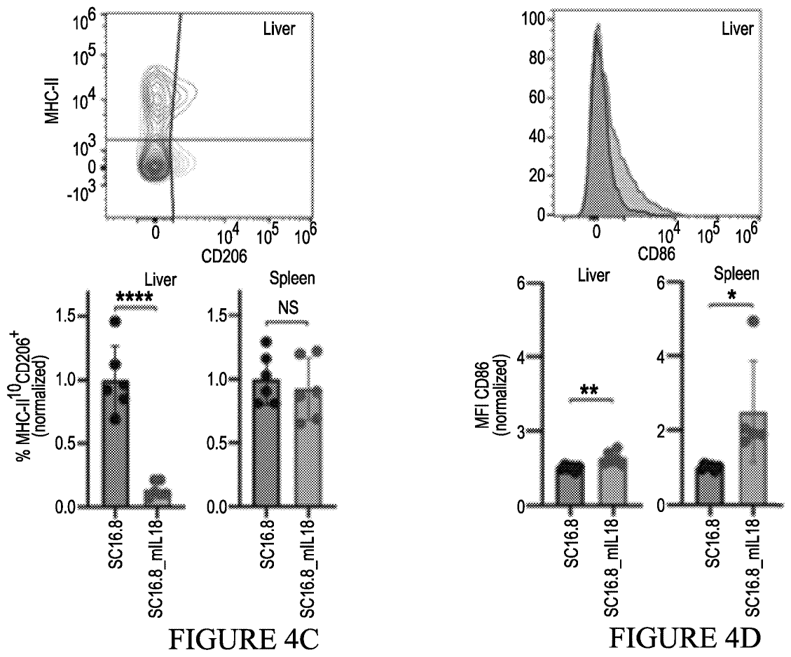

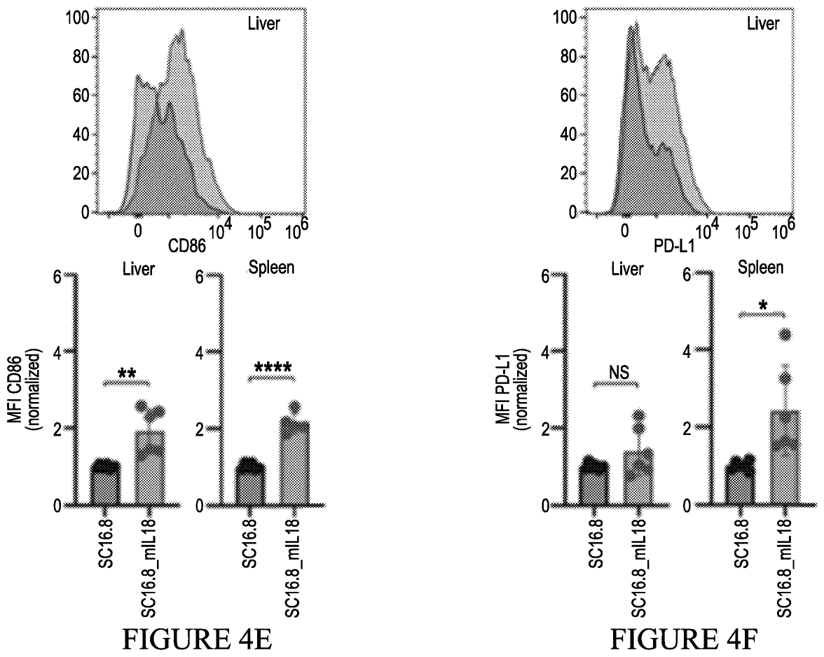

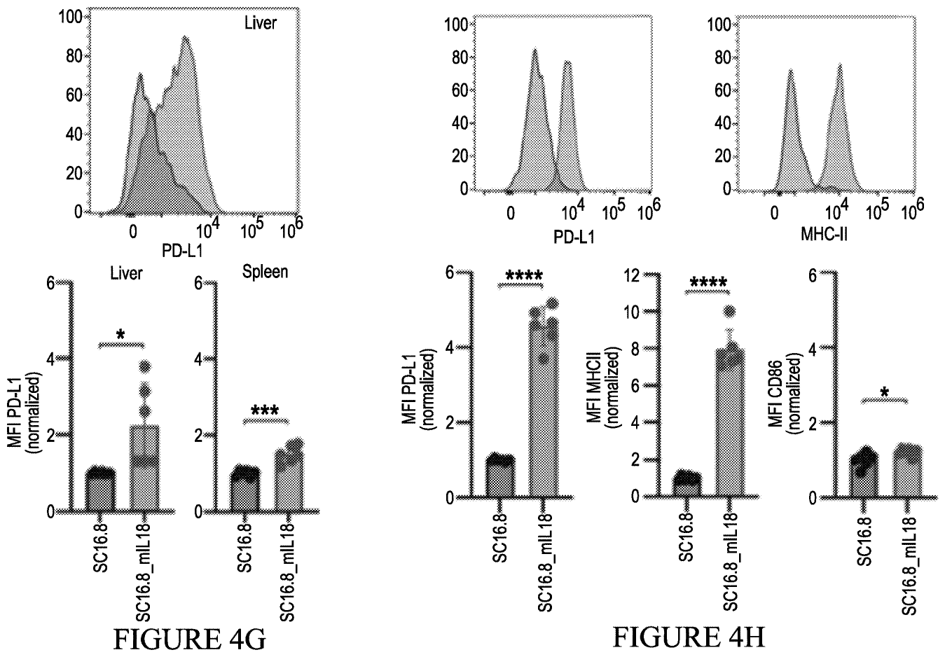

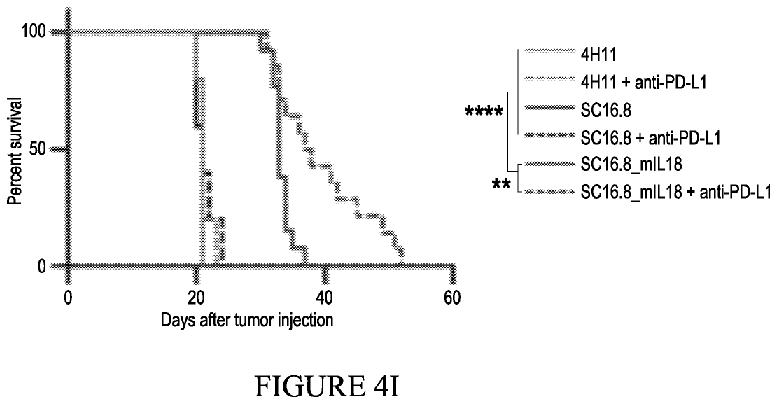

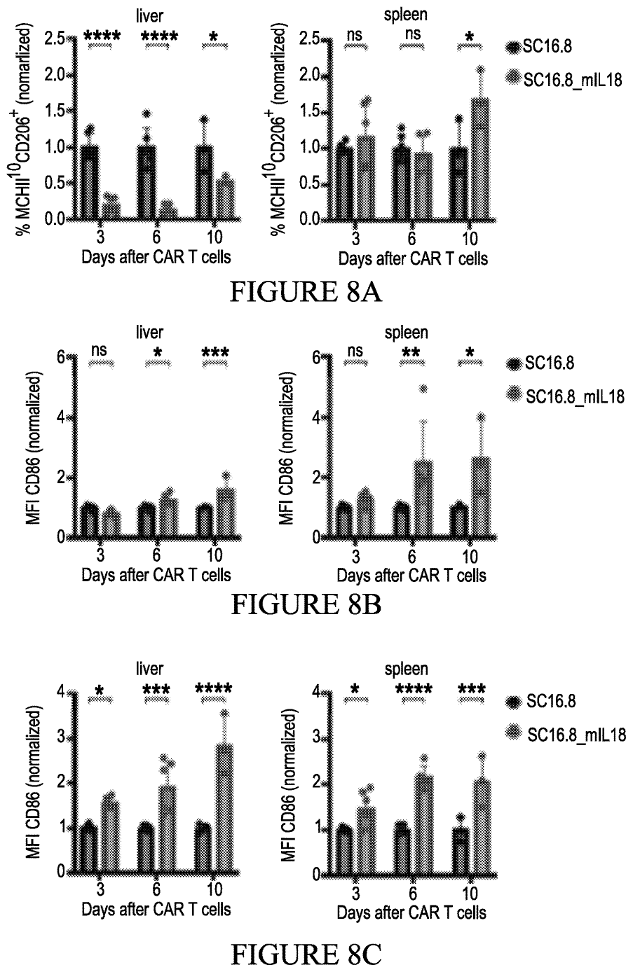

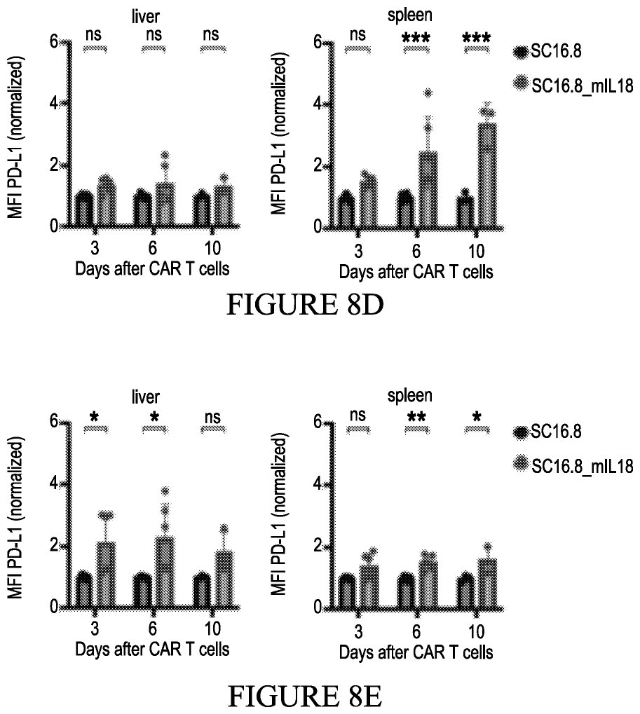

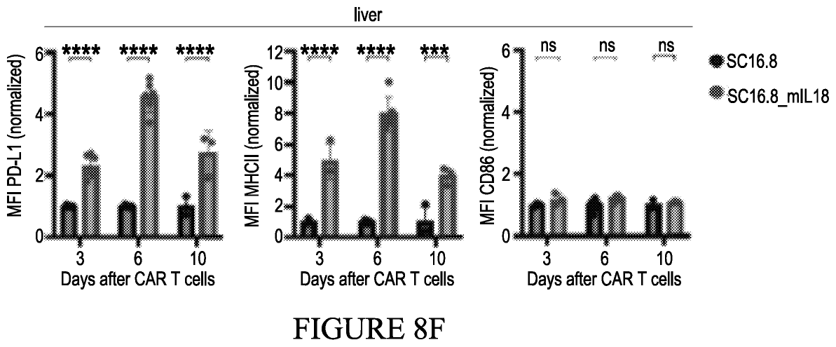

Next, the presently disclosed subject matter analyzed the TME and spleen for levels and activation of macrophages and dendritic cells on the same days as described in FIG. 3A and observed an increase in both CD1 lb+Gr1− macrophages and CD11c⁺MHC-II+ dendritic cells of live CD45+ cells in tumor-bearing livers of mice treated with mIL-18-secreting CAR T cells, compared with CAR T cells without mIL-18 (FIG. 4A). No significant changes in prevalence of either cell type were seen in the corresponding spleens of these animals (FIG. 4B). Importantly, liver macrophages of mice treated with mIL-18-secreting CAR T cells displayed a reduction in the CD206⁺MHC-IIlo “M2-like” antiinflammatory phenotype (FIG. 4C and FIG. 8A). Macrophages and dendritic cells in both the liver and spleen expressed higher levels of activation marker CD86 (FIG. 4, D and E, and FIG. 7, B and C), indicating a systemic activation of antigen-presenting cells. IFN-γ is known to induce PD-L1 expression. In line with the previous observation that mIL-18 secretion leads to higher IFN-γ levels in the serum (FIG. 2G), there was an increase in PD-L1 levels on macrophages and dendritic cells following treatment with mIL-18-secreting CAR T cells (FIGS. 4F and 4G, and FIGS. 7D and 7E). This increase in PD-L1 was especially profound on a subset of macrophages that express F4/80 (FIG. 4H and FIG. 7F). The F4/80+ macrophages did not increase in number but were highly activated with increased expression of MHC-II and CD86 (FIG. 4H and FIG. 7F).

PD-L1 expression in the TME, on tumor cells, or both, can hamper an effective CAR T cell antitumor response. Therefore the potential benefit of blocking the inhibitory PD-L1/PD-1 interaction by combining CAR T cell treatment with anti-PD-L1 antibody therapy was assessed. Tumor-bearing mice received 3 doses of 300 μg anti-PD-L1 antibody starting 1 week after CAR T cell treatment. A significant survival improvement was observed when mIL-18-secreting CAR T cells were combined with anti-PD-L1 antibodies, whereas anti-PD-L1 therapy had no effect on survival following administration of CAR T cells that did not secrete mIL-18 (FIG. 4I).

In conclusion, the presently disclosed subject matter demonstrates that the secretion of mIL-18 by DLL3-targeted CAR T cells leads to an activated, more proinflammatory myeloid compartment, both in the TME and systemically. The antitumor response can be further improved by blocking inhibitory effects of mIL-18-induced PD-L1 with antibodies.

IL-18-Secreting Human DLL3-Targeting CAR T Cells Cure Mice with Metastatic SCLC.

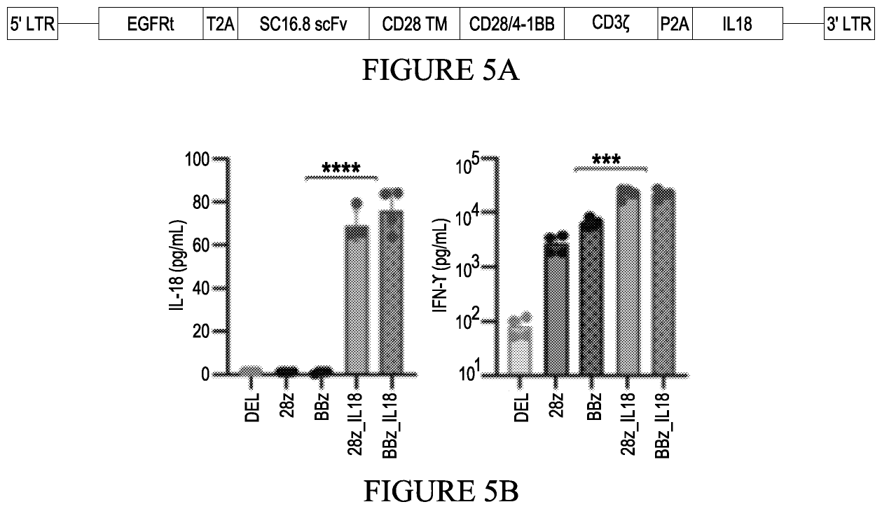

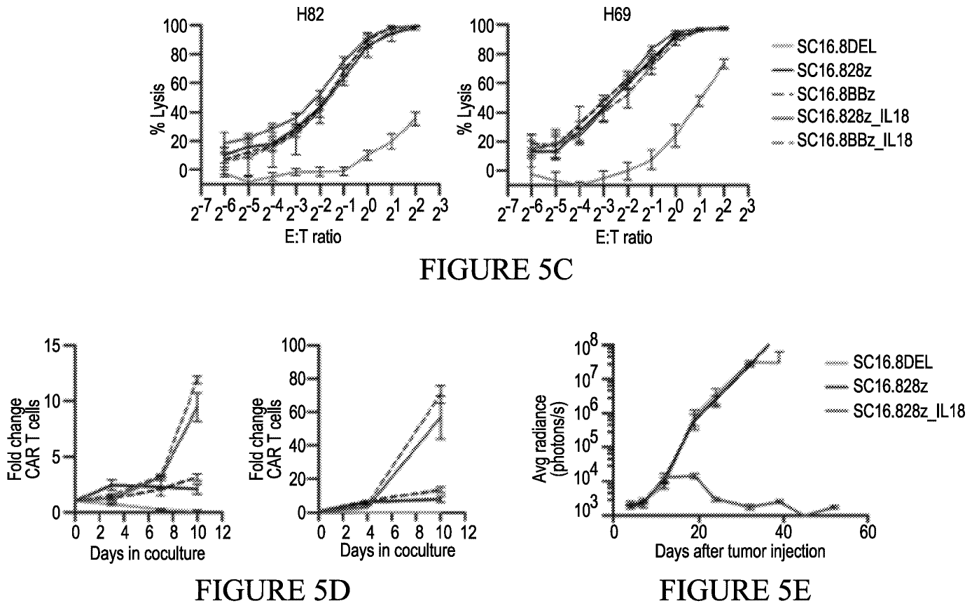

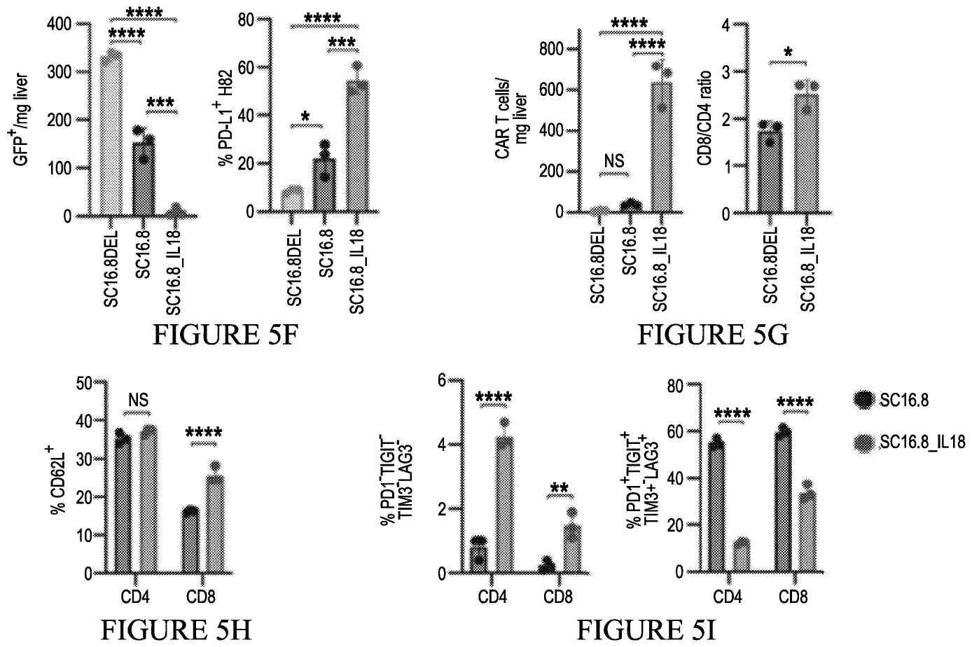

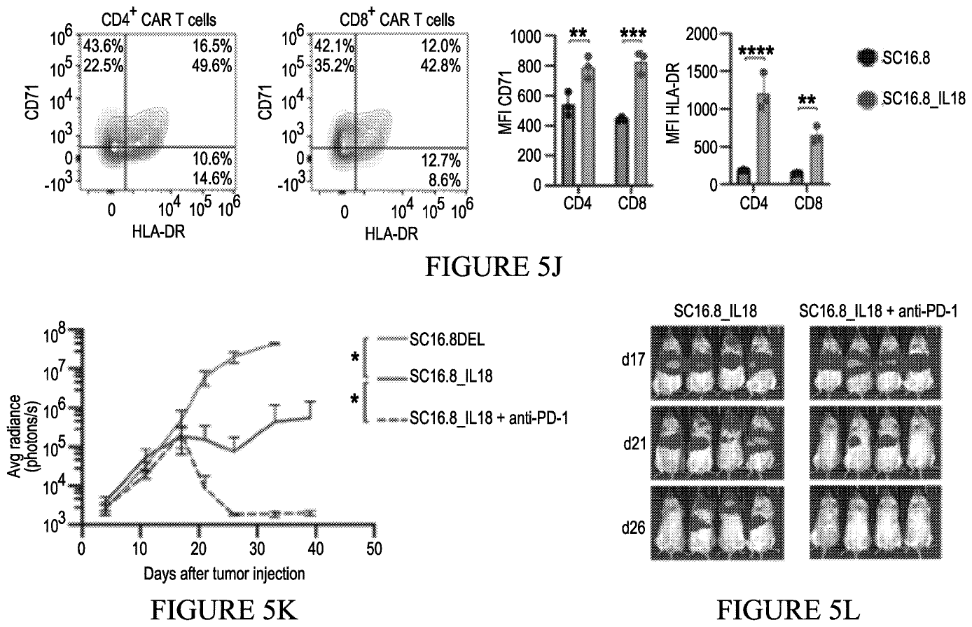

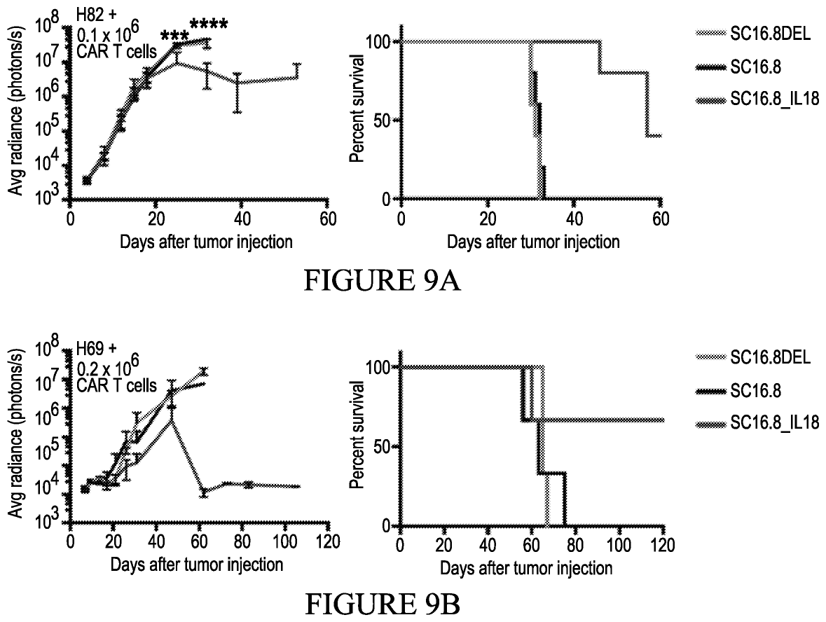

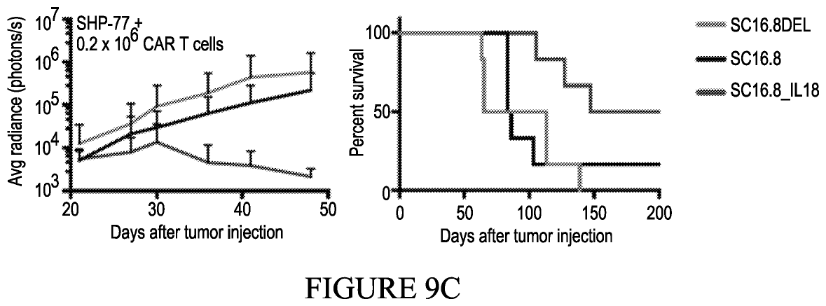

After having observed a marked improvement in the efficacy of murine DLL3-targeting CAR T cells against systemic mSCLC in immunocompetent mice with CAR T cells secreting mIL-18, the presently disclosed subject matter sought to test the potential benefit of IL-18 on human CAR T cells in xenograft SCLC models. First, human IL18 transgene was cloned into a retroviral vector containing either the CD28 or the 4-1BB costimulatory domain and with a truncated EGFR for detection of transduced T cells (FIG. 5A). The SC16.8DEL construct, which lacks the intracellular CD28/4-1BB and CD3ζ domains, was used as negative control. In the supernatant of cocultures with CAR T cells and DLL3-expressing cells, the IL-18 expression as well as increased IFN-γ levels were validated (FIG. 5B). Lysis potential of the CAR T cells was similar, regardless of IL-18 secretion or costimulation with both H82 (DLL3+) and H69 (DLL3^{lo}) SCLC cells (FIG. 5C). In clinical trials, CAR T cell proliferation and expansion has proven an important hallmark of successful CAR T cell therapy. When the SC16.8 CAR T cells were cocultured with H82 or H69 SCLC cells (at an effector-to-target [E/T] ratio of 1:5), secreted IL-18 greatly enhanced the CAR T cell expansion in vitro (FIG. 5D). Given the similarity in evident activity with either the CD28 or 4-1BB coactivation domains, the activity of the CD28 construct was further explored. Consistent with the augmentation of CAR T cell activity provided by endogenous IL-18 production, in vivo administration of 1×10⁶ SC16.8 CAR T cells failed to elicit an antitumor response, whereas the SC16.8_IL18 CAR T cells were able to fully eradicate H82 tumors (FIG. 5E). Low doses of SC16.8_IL18 CAR T cells were able to prolong survival or induce long-term responses in SCLC models with metastatic H82 and H69 tumors and orthotopic SHP-77 tumors (FIG. 9).

The presently disclosed subject matter next sought to further characterize the effects of IL-18 on human CAR T cell activation and phenotype in vivo. H82 tumor cells were infused 9 days prior to administration of 2×10⁶ CAR T cells, and livers (the main tumor location) were collected for flow cytometric analysis 9 days after treatment. The tumor burden was reduced in SC16.8 CAR T cell-treated mice, and it was even further reduced when treated with SC16.8_IL18 CAR T cells. Higher levels of PD-L1 were detected on the remaining tumor cells (FIG. 5F). The number of CAR T cells detected in the liver was dramatically increased in the presence of IL-18. SC16.8DEL CAR T cell levels were so low that they could not be analyzed further. IL-18 secretion led to an increase in the CD8+/CD4+ CAR T cell ratio (FIG. 5G). CD8+ CAR T cells displayed higher levels of the memory T cell marker CD62L (FIG. 5H). The combination of 4 exhaustion markers (PD-1, TIGIT, LAG3, TIM3) was used to distinguish nonexhausted (quadruple negative) and fully exhausted (quadruple positive) CAR T cells. The secretion of IL-18 resulted in higher levels of nonexhausted and lower levels of fully exhausted CAR T cells (FIG. 5I). The T cell activation markers CD71 and HLA-DR were more highly expressed on SC16.8_IL18 CAR T cells than on SC16.8 CAR T cells (FIG. 5J).

Finally, the presently disclosed subject matter tested whether it could further increase the antitumor efficacy of SC16.8_IL18 CAR T cells with additional ICB. Mice with metastatic H82 tumors received a suboptimal dose of 0.3×10⁶ SC16.8_IL18 CAR T cells and were treated with 250 μg anti-PD-1 antibody twice weekly, starting 2 weeks after CAR T cell infusion, further improving the antitumor response of SC16.8_IL18 CAR T cells (FIG. 5, K and L). These data, in a mouse lacking endogenous T cells, suggest a capacity for PD-1 blockade to further augment the efficacy of the anti-DLL3, IL-18-secreting CAR T cells directly.

All together, these results demonstrate that the secretion of IL-18 by SC16.8 CAR T cells increases a memory phenotype and T cell activation, reduces exhaustion, and improves the antitumor efficacy, which can be further enhanced by ICB in vivo.

CAR T cell therapies tested in patients with solid cancers have not proven to be highly effective, in contrast to hematological malignancies, for which 6 CAR T cell products are FDA approved (4 targeting CD19 and 2 targeting BCMA). Possible explanations for CAR T cell failure include an immunosuppressive TME leading to T cell exhaustion and heterogeneity of target antigen expression. The presently disclosed IL-18-secreting CAR T cells against DLL3 address both of these issues. The presently disclosed subject matter, developed a CAR based on an anti-DLL3 SC16 antibody that is effective in various metastatic and orthotopic SCLC models with medium (H82 and SHP-77) and low (H69) surface expression levels of DLL3. While not wishing to be bound to any particular theory, one may expect that proinflammatory cytokines IL-12 and IL-18, which are both normally produced by myeloid cells, may stimulate CAR T cell activation and alter the TME to a less immunosuppressive state. Thus, different cytokines may be optimal in the context of different tumor types. The presently disclosed CAR was cross-reactive to murine DLL3 and enabled the study of the effect of CAR T cell-secreted IL-12 and IL-18 in a syngeneic immunocompetent mSCLC model. In this direct comparison, IL-18 stimulated a more robust antitumor response than IL-12, which prompted the further characterization of IL-18-induced changes to the TME.

In addition to enhanced activation of the CAR T cells themselves, endogenous tumor-infiltrating lymphocytes (TILs) were more activated in the presence of IL-18. Together with the increased antitumor TIL response in the ELISpot assay and delayed tumor outgrowth after rechallenge with DLL3KO mSCLC tumors, this indicates that IL-18 stimulates tumor-reactive TILs, and is important for the elimination of DLL3⁻ tumor cells in tumors with heterogeneous DLL3 expression. In addition to its effects on the TME, IL-18 strongly stimulated the proliferation, persistence, and memory phenotype of human DLL3 CAR T cells and reduced their exhaustion, leading to long-term remission in 3 different SCLC models. IL-18 is a potent inducer of IFN-γ production, which was observed in the sera of SC16.8_mIL18 CAR T cell-treated mice and in supernatant of human SC16.8_IL18 CAR T cells cocultured with DLL3-expressing target cells. IFN-γ, however, led to upregulation of the inhibitory ligand PD-L1 on both tumor cells and myeloid effector cells. Combination treatment of a suboptimal dose of IL-18-secreting CAR T cells with delayed ICB improved the CAR T cell response. ICB is approved for SCLC and integrated in first-line treatment. Thus, the benefits and timing of ICB after CAR T cell treatment can be determined in clinical trials. Given the typically poor clinical outcome, SCLC is in dire need of more targeted treatment options and CAR T cells are a valuable addition. After preconditioning chemotherapy, which is routinely given prior to CAR T cell infusion, the SC16.8_mIL18 CAR T cells induced long-term remission in an aggressive immunocompetent metastatic mSCLC model.

DLL3 is expressed on a wide variety of neuroendocrine cancers, including high-grade neuroendocrine cancers of the lung, prostate, breast, pancreas, and intestinal tract as well as low-grade glioma and neuroblastoma. Neuroendocrine cancers often lack therapeutic options and most have a poor prognosis. The presently disclosed potent, DLL3-targeting CAR T cells serve as an important addition to the treatment armamentarium for patients with SCLC and other DLL3+ neuroendocrine cancers.

### Methods

Cell lines. H82 (ATCC, HTB-175), H69 (ATCC, HTB-119), and SHP-77 (ATCC, CRL-2195) SCLC cells and murine Trp53^{-/-}; Rb^{-/-} mSCLC cells were stably transfected with a retroviral vector expressing GFP and luciferase and were maintained in RPMI1640 medium (Gibco) supplemented with 10% heat-inactivated FBS, L-glutamine, and penicillin/streptomycin. Galv9 293 and Phoenix ecotropic (ATCC, CRL-3214) packaging cells were maintained in DMEM (Gibco) supplemented with 10% FBS, L-glutamine, and penicillin/streptomycin.

Animal models and treatments. Six- to eight-week-old male NSG (NOD.Cg-Prkdc^{scid}IL2rg^{tm1Wj1}/SzJ) mice were purchased from The Jackson Laboratory (no. 005557) and used for all xenograft experiments. For the metastatic SCLC models, 1×10⁶ H82 or H69 cells in 200 L PBS were injected into the tail vein. For the orthotopic SCLC models, 1×10⁶ H82 or SHP-77 cells in 40 L PBS were injected intrathoracically. Tumor size was measured with bioluminescence for randomization before CAR T cell transfer and then once or twice per week to monitor tumor growth. Anti-PD-1 antibody (BioLegend, clone EH12.2H7, 250 μg i.p.) was given twice weekly for 3 weeks, starting 2 weeks after CAR T cell administration.

Six- to eight-week-old female immunocompetent B6129SF1/J mice (The Jackson Laboratory, no. 101043) were used for all experiments with mSCLC cells. 1×10⁶ mSCLC cells were injected in 200 L PBS into the tail vein, and 7 days later mice received 2×10⁶ (unless indicated otherwise) murine CAR T cells. When indicated, mice received 1 i.p. injection of 50 mg/kg cyclophosphamide 1 day prior to CAR T cell transfer. Tumor size was measured with bioluminescence for randomization before CAR T cell treatment and then once or twice per week to monitor tumor growth. Anti-PD-L1 antibody (BioXCell, clone 10F.9G2, 300 μg i.p.) was given on days 14, 17, and 20 after tumor injection.

For the rechallenge with wild-type or DLL3KO mSCLC cells, B6129SF1/J mice received 1×10⁶ mSCLC cells via tail vein (day 0), 50 mg/kg cyclophosphamide i.p. (day 6), and 0.5×10⁶ SC16.8_mIL18 CAR T cells via tail vein (day 7). On day 42, mice without detectable tumor (~75% of cohort) and age-matched naive control mice received 1×10⁶ mSCLC or mSCLC-DLL3KO cells via tail vein.

Generation of retroviral constructs. Plasmids encoding the human and mouse CAR constructs in a SFG γ-retroviral vector were used to transfect gpg29 fibroblasts (H29) with the ProFection Mammalian Transfection System (Promega) according to manufacturer's instructions. The retroviral supernatants were used for transduction of Galv9 293 or Phoenix ECO cell lines to generate stable retroviral particle-producing cell lines. All vectors were generated by restriction enzyme digest and Gibson Assembly (New England BioLabs).

The human CARs used human EGFR cDNA without the intracellular domain, a T2A self-cleaving peptide, cDNA of the scFvs, the Flag-tag sequence (DYKDDDDK), the human CD28 transmembrane and intracellular domain, the human 4-1BB intracellular domain, the human CD3ζ intracellular domain, a P2A self-cleaving peptide, and the mature, processed form of human IL-18. The murine CARs used mCherry, a T2A self-cleaving peptide, cDNA of the SC16.8 scFv with Flag-tag, the murine CD28 transmembrane and intracellular domain, the murine CD3ζ intracellular domain, an internal ribosome entry site, a fusion gene encoding the complete murine IL-12 with a serine-glycine repeat between the p35 and p40 chain-coding domains, a P2A self-cleaving peptide, and the mature form of murine IL-18.

T cell isolation and retroviral transduction. Human and mouse T cells were isolated, activated, and transduced. In brief, peripheral blood mononuclear cells were isolated from leukopacks (New York Blood Center, New York, New York, USA), from which T cells were isolated with a EasySep Human T Cell Isolation Kit (Stemcell Technologies). T cells were activated with CD3/CD28 Dynabeads (bead/cell ratio of 1:5) and 100 IU/mL IL-2 (Peprotech) and cultured in RPMI1640 supplemented with 10% FBS, 2 mM L-glutamine, and 1% penicillin/streptomycin. After 48 hours, CD3/CD28 beads were removed, and T cells were transduced by spinoculation on plates coated with retronectin (Takara Clontech) with retroviral supernatant from viral packaging cells. Mouse T cells were isolated from spleens, following red blood cell lysis, with a nylon wool fiber column (Polysciences). T cells were activated with CD3/CD28 Dynabeads (bead/cell ratio of 1:2) and 100 IU/mL IL-2 and cultured in RPMI1640 supplemented with 10% FBS, 2 mM L-glutamine, nonessential amino acids, sodium pyruvate, HEPES, 2-mercaptoethanol, and 1% penicillin/streptomycin. After 24 and 48 hours, T cells were transduced by spinoculation on retronectin-coated plates in viral supernatant from Phoenix ECO cells. T cells were rested for 1 day and then used for in vitro and in vivo assays.

Cytotoxicity assays. Mouse or human CAR T cells were cocultured at varying E/T ratios with 5×10⁴ firefly luciferase-expressing tumor cells in a 96-well plate in 200 L media for 20 hours. 75 ng D-luciferin (Gold Biotechnologies) in 5 μL PBS was added to each well and luminescence was measured with a Tecan Sparke microplate reader (Tecan). Wells with target cells alone were used to determine the “max signal.” Percentage lysis was calculated as [1−(“sample signal”/“max signal”)]×100.

Proliferation assays. 1×10⁵ human CAR T cells were cocultured in a 12-well plate with 5×10⁵ target cells on day 0. At the indicated time points, each well was resuspended and 20 L was taken for quantification of CAR T cells by flow cytometry using volume analysis with the Attune NxT flow cytometer (Thermo Fisher Scientific).

Cytokine analysis. For analysis of in vitro cytokine secretion, 1×10⁵ CAR T cells were cocultured with 1×10⁵ target cells in a 96-well plate in 200 L media. After 24 hours, the supernatant was collected. For serum cytokine analysis, whole blood was collected and serum was prepared by allowing the blood to clot by centrifuging at 14,000 g for 15 minutes at 4° C. Samples were analyzed with Milliplex Map Human cytokine/chemokine, Premixed 12-plex kit or the Milliplex Map Mouse cytokine/chemokine, Premixed 10-plex kit, for human and mouse cytokines respectively, and the FlexMap 3D system on a Luminex IS100 system (Millipore).

Flow cytometry. A Gallios B43618 (Beckman Coulter) and Attune NxT (Thermo Fisher Scientific) were used for flow cytometric data acquisition, and analysis was performed with FlowJo software. DAPI (0.5 mg/mL, Sigma-Aldrich) or a LIVE/DEAD fixable yellow dead cell stain kit (Thermo Fisher Scientific) was used to exclude dead cells in all experiments. CAR expression was detected with anti-Flag (L5), Cetuximab-A647, or mCherry. Tumor cells were detected with EGFP. DLL3, both human and mouse, was detected with a SC16.34-PE antibody (provided by AbbVie). Nonspecific binding of antibodies was blocked with anti-mouse CD16/CD32 antibody. The following anti-mouse antibodies were used: anti-CD45 (clone 30F11, BioLegend), anti-CD3ε (145-2C11, eBioscience), anti-CD4 (GK1.5, eBioscience), anti-CD8α (53-6.7, eBioscience), anti-TNF-α (MP6-XT22, BioLegend), anti-IFN-γ (XMG1.2, BioLegend), anti-CD11b (M1/70, eBioscience), anti-Gr1 (RB6-8C5, BioLegend), anti-CD11c (N418, eBioscience), anti-MHC-III-A/I-E (M5/114.15.2, BioLegend), anti-CD206 (MMR, eBioscience), anti-F4/80 (BM8, eBioscience), anti-CD86 (GL1, BioLegend), and anti-PD-L1 (1° F.9G2, BioLegend). For intracellular staining of TNF-α and IFN-γ, single-cell suspensions from livers and spleens were stimulated with ix Cell Stimulation Cocktail (phorbol 12-myristate 13-acetate, ionomycin, brefeldin A, and monensin) from Thermo Fisher Scientific for 4 hours. Cells were then processed with the Cytofix/Cytoperm Plus kit (BD Biosciences). The following anti-human antibodies were used: anti-CD45 (30-F11, BioLegend), anti-PD-L1 (29E.2A3, Bio-Legend), anti-CD4 (SK3, eBioscience), anti-CD8 (SK1, BD Biosciences), anti-CD71 (CY1G4, BioLegend), anti-HLA-DR (L243, BioLegend), anti-PD-1 (EH12.2H7, BioLegend), anti-TIGIT (A15153G, BioLegend), anti-TIM3 (F38-2E2, BioLegend), anti-LAG3 (11C3C65, BioLegend), and anti-CD62L (DREG-56, BioLegend).

Cells were isolated from livers and spleens and the prepared for flow cytometry. For analysis of circulating CAR T cells, whole blood was collected and spun at 14,000 g for 15 minutes at 4° C. Red blood cell lysis was done with an ammonium-chloride-potassium Lysing Buffer (Lonza). Cells were washed with PBS and used for flow cytometric analysis.

ELISpot assay. A single-cell suspension of the liver of mice treated with 2×10⁶ CAR T cells was prepared and subjected to fluorescence-activated cell sorting (using a BD Aria cell sorter). mSCLC cells were irradiated with 30 Gy using a GE Mark1 cesium irradiator, and 1×10⁵ cells were plated per well in a mIFN-γ ELISpot plate (Millipore ELISpot plates, Mabtech). Sorted T cell populations were added at a 1:1 effector-to-target ratio and incubated at 37° C. overnight. After 24 hours, the plate was developed according to manufacturer's instructions and analyzed with ImmunoSpot software.

Generation of DLL3 knock out cells. HEK-293FT cells were transfected using X-tremeGene HP (Sigma Aldrich) with a LentiCRISPRv2 vector encoding Cas9, sgRNA (CGGCTCGCACCGCGCGCGGT, SEQ ID NO: 151), and a puromycin resistance gene and the packaging plasmids psPAX2 and pMD2.G. HEK-293FT supernatants from 48 and 72 hours after transfection were pooled and used for transduction of mSCLC cells. After puromycin selection, single-cell clones were generated and upon outgrowth tested for DLL3 expression by flow cytometry.

Statistics. All statistical analyses were performed with Graph-Pad Prism software. Statistical significance was determined using an unpaired, 2-tailed Student's t test, 1-way ANOVA, or 2-way ANOVA, as indicated in the figure legends. The log-rank (Mantel-Cox) test was used to determine statistical significance for overall survival in mouse survival experiments. Data are shown as the mean±SD. P values of less than 0.05 were considered significant.

Study approval. Mice were housed under specific pathogen-free conditions in the animal facility of MSKCC, and all experiments were performed in accordance with MSKCC Institutional Animal Care and Use Committee-approved protocol guidelines (MSKCC 00-05-065, 13-07-007, and 18-06-009).

### EMBODIMENTS OF THE PRESENTLY DISCLOSED SUBJECT MATTER

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A cell comprising
a) an antigen-recognizing receptor comprises an extracellular antigen-binding domain, a transmembrane domain, and an intracellular signaling domain;
b) an exogenous IL-18 polypeptide;
wherein the extracellular antigen-binding domain specifically binds to DLL3, wherein the extracellular antigen-binding domain comprises:
i) a heavy chain variable region comprising a CDR1 comprising the amino acid sequence set forth in SEQ ID NO: 1, a CDR2 comprising the amino acid sequence set forth in SEQ ID NO: 2, and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6;
ii) a heavy chain variable region comprising a CDR1 comprising the amino acid sequence set forth in SEQ ID NO: 11, a CDR2 comprising an amino acid sequence set forth in SEQ ID NO: 12, and a CDR3 comprising the amino acid sequence set forth in SEQ ID NO: 13; and a light chain variable region comprising a CDR1 comprising the amino acid sequence set forth in SEQ ID NO: 14, a CDR2 comprising the amino acid sequence set forth in SEQ ID NO: 15, and a CDR3 comprising the amino acid sequence set forth in SEQ ID NO: 16;
iii) a heavy chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23; and a light chain variable region comprising a CDR1 comprising the amino acid sequence set forth in SEQ ID NO: 24, a CDR2 comprising the amino acid sequence set forth in SEQ ID NO: 25, and a CDR3 comprising the amino acid sequence set forth in SEQ ID NO: 26;
iv) a heavy chain variable region comprising a CDR1 comprising the amino acid sequence set forth in SEQ ID NO: 11, a CDR2 comprising the amino acid sequence set forth in SEQ ID NO: 31, and a CDR3 comprising the amino acid sequence set forth in SEQ ID NO: 32; and a light chain variable region comprising a CDR1 comprising the amino acid sequence set forth in SEQ ID NO: 25, a CDR2 comprising the amino acid sequence set forth in SEQ ID NO: 33, and a CDR3 comprising the amino acid sequence set forth in SEQ ID NO: 34;
v) a heavy chain variable region comprising a CDR1 comprising the amino acid sequence set forth in SEQ ID NO: 39, a CDR2 comprising the amino acid sequence set forth in SEQ ID NO: 40, and a CDR3 comprising the amino acid sequence set forth in SEQ ID NO: 41, and a light chain variable region comprising a CDR1 comprising the amino acid sequence set forth in SEQ ID NO: 42, a CDR2 comprising the amino acid sequence set forth in SEQ ID NO: 43, and a CDR3 comprising the amino acid sequence set forth in SEQ ID NO: 44;
vi) a heavy chain variable region comprising a CDR1 comprising the amino acid sequence set forth in SEQ ID NO: 49, a CDR2 comprising the amino acid sequence set forth in SEQ ID NO: 50, and a CDR3 comprising the amino acid sequence set forth in SEQ ID NO: 51; and a light chain variable region comprising a CDR1 comprising the amino acid sequence set forth in SEQ ID NO: 52, a CDR2 comprising the amino acid sequence set forth in SEQ ID NO: 53, and a CDR3 comprising the amino acid sequence set forth in SEQ ID NO: 54;
vii) a heavy chain variable region comprising a CDR1 comprising the amino acid sequence set forth in SEQ ID NO: 59, a CDR2 comprising the amino acid sequence set forth in SEQ ID NO: 60, and a CDR3 comprising the amino acid sequence set forth in SEQ ID NO: 61; and a light chain variable region comprising a CDR1 comprising the amino acid sequence set forth in SEQ ID NO: 62, a CDR2 comprising the amino acid sequence set forth in SEQ ID NO: 63, and a CDR3 comprising the amino acid sequence set forth in SEQ ID NO: 64; or
viii) a heavy chain variable region comprising a CDR1 comprising the amino acid sequence set forth in SEQ ID NO: 69, a CDR2 comprising the amino acid sequence set forth in SEQ ID NO: 70, and a CDR3 comprising the amino acid sequence set forth in SEQ ID NO: 71; and a light chain variable region comprising a CDR1 comprising the amino acid sequence set forth in SEQ ID NO: 62, a CDR2 comprising the amino acid sequence set forth in SEQ ID NO: 72, and a CDR3 comprising the amino acid sequence set forth in SEQ ID NO: 73.

2. The cell of claim 1, wherein the extracellular antigen-binding domain comprises:
a) a heavy chain variable region comprising a CDR1 comprising the amino acid sequence set forth in SEQ ID NO: 1, a CDR2 comprising the amino acid sequence set forth in SEQ ID NO: 2 and a CDR3 comprising the amino acid sequence set forth in SEQ ID NO: 3; and a light chain variable region comprising a CDR1 comprising the amino acid sequence set forth in SEQ ID NO: 4, a CDR2 comprising the amino acid sequence set forth in SEQ ID NO: 5, and a CDR3 comprising the amino acid sequence set forth in SEQ ID NO: 6; or
b) a heavy chain variable region comprising a CDR1 comprising the amino acid sequence set forth in SEQ ID NO: 69, a CDR2 comprising an amino acid sequence set forth in SEQ ID NO: 70, and a CDR3 comprising the amino acid sequence set forth in SEQ ID NO: 71; and a light chain variable region comprising a CDR1 comprising the amino acid sequence set forth in SEQ ID NO: 62, a CDR2 comprising the amino acid sequence set forth in SEQ ID NO: 72, and a CDR3 comprising the amino acid sequence set forth in SEQ ID NO: 73.

3. The cell of claim 1, wherein the extracellular antigen-binding domain is a single-chain variable fragment (scFv), a Fab, or a F(ab)2.

4. The cell of claim 1, wherein the extracellular antigen-binding domain comprises a heavy chain variable region comprising the amino acid sequence set forth in SEQ ID NO: 7, SEQ ID NO: 17, SEQ ID NO: 27, SEQ ID NO: 35, SEQ ID NO: 45, SEQ ID NO: 55, SEQ ID NO: 65, or SEQ ID NO: 74.

5. The cell of claim 1, wherein the extracellular antigen-binding domain comprises a light chain variable region comprising the amino acid sequence set forth in SEQ ID NO: 8, SEQ ID NO: 18, SEQ ID NO: 28, SEQ ID NO: 36, SEQ ID NO: 46, SEQ ID NO: 56, SEQ ID NO: 66, or SEQ ID NO: 75.

6. The cell of claim 1, wherein the extracellular antigen-binding domain comprises:
a) a heavy chain variable region comprising the amino acid sequence set forth in SEQ ID NO: 7, SEQ ID NO: 17, SEQ ID NO: 27, SEQ ID NO: 35, SEQ ID NO: 45, SEQ ID NO: 55, SEQ ID NO: 65, or SEQ ID NO: 74; and
b) a light chain variable region comprising the amino acid sequence set forth in SEQ ID NO: 8, SEQ ID NO: 18, SEQ ID NO: 28, SEQ ID NO: 36, SEQ ID NO: 46, SEQ ID NO: 56, SEQ ID NO: 66, or SEQ ID NO: 75.

7. The cell of claim 1, wherein the extracellular antigen-binding domain comprises:
a) a heavy chain variable region comprising the amino acid sequence set forth in SEQ ID NO: 7, and a light chain variable region comprising the amino acid sequence set forth in SEQ ID NO: 8;
b) a heavy chain variable region comprising the amino acid sequence set forth in SEQ ID NO: 17, and a light chain variable region comprising the amino acid sequence set forth in SEQ ID NO: 18;
c) a heavy chain variable region comprising the amino acid sequence set forth in SEQ ID NO: 27, and a light chain variable region comprising the amino acid sequence set forth in SEQ ID NO: 28;
d) a heavy chain variable region comprising the amino acid sequence set forth in SEQ ID NO: 35, and a light chain variable region comprising the amino acid sequence set forth in SEQ ID NO: 36;
e) a heavy chain variable region comprising the amino acid sequence set forth in SEQ ID NO: 45, and a light chain variable region comprising the amino acid sequence set forth in SEQ ID NO: 46;
f) a heavy chain variable region comprising the amino acid sequence set forth in SEQ ID NO: 55, and a light chain variable region comprising the amino acid sequence set forth in SEQ ID NO: 56;
g) a heavy chain variable region comprising the amino acid sequence set forth in SEQ ID NO: 65, and a light chain variable region comprising the amino acid sequence set forth in SEQ ID NO: 66; or
h) a heavy chain variable region comprising the amino acid sequence set forth in SEQ ID NO: 74, and a light chain variable region comprising the amino acid sequence set forth in SEQ ID NO: 75.

8. The cell of claim 1, wherein the extracellular antigen-binding domain comprises a linker between a heavy chain variable region and a light chain variable region of the extracellular antigen-binding domain.

9. The cell of claim 1, wherein the extracellular antigen-binding domain comprises a signal peptide that is covalently joined to the 5′ terminus of the extracellular antigen-binding domain.

10. The cell of claim 1, wherein the transmembrane domain comprises a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, or a combination thereof.

11. The cell of claim 1, wherein the intracellular signaling domain comprises a CD3ζ polypeptide.

12. The cell of claim 1, wherein the intracellular signaling domain further comprises at least one co-stimulatory signaling region.

13. The cell of claim 1, wherein the antigen-recognizing receptor is a chimeric antigen receptor (CAR), or a T-cell like fusion protein.

14. The cell of claim 1, wherein the exogenous IL-18 polypeptide is a human IL-18 polypeptide.

15. The cell of claim 1, wherein the cell is selected from the group consisting of a T cell, a Natural Killer (NK) cell, and a stem cell from which a lymphoid cell may be differentiated.

16. The cell of claim 15, wherein the T cell is a cytotoxic T lymphocyte (CTL) or a regulatory T cell.

17. The cell of claim 15, wherein the stem cell is a pluripotent stem cell, an embryoid stem cell, or an induced pluripotent stem cell.

18. A method of treating or ameliorating a disease or disorder in a subject, comprising administering to the subject the presently disclosed cell of claim 1.

19. The method of claim 18, wherein the disease or disorder is selected from the group consisting of neuroendocrine tumors of the lung, pulmonary neuroendocrine cancer, large cell neuroendocrine carcinoma, and small-cell lung cancer, extrapulmonary neuroendocrine carcinomas, melanoma, neuroendocrine prostate cancer, breast cancer, neuroendocrine tumors of the gastrointestinal tract, pancreatic cancer, medullary thyroid cancer, small cell bladder cancer, ovarian small cell carcinoma, low-grade glioma, glioblastoma and neuroblastoma.

20. A kit for treating or ameliorating a disease or disorder in a subject, comprising the cell of claim 1.


SEQUENCE LISTING (151 sequences)

SEQ ID NO: 1 - protein, 8 aa, synthetic construct
  REGION 1..8: note = Description of sequence: component of antibody: SC16.8 VH CDR1
GYAFTNYL

SEQ ID NO: 2 - protein, 8 aa, synthetic construct
  REGION 1..8: note = Description of sequence: component of antibody: SC16.8 VH CDR2
INPGTGGT

SEQ ID NO: 3 - protein, 25 aa, synthetic construct
  REGION 1..25: note = Description of sequence: component of antibody: SC16.8 VH CDR3
ARSPYDYHEGAMDYWGQGTSVTVSS

SEQ ID NO: 4 - protein, 9 aa, synthetic construct
  REGION 1..9: note = Description of sequence: component of antibody: SC16.8 VL CDR1
QATQDIVKN

SEQ ID NO: 5 - protein, 7 aa, synthetic construct
  REGION 1..7: note = Description of sequence: component of antibody: SC16.8 VL CDR2
YAIELAE

SEQ ID NO: 6 - protein, 19 aa, synthetic construct
  REGION 1..19: note = Description of sequence: component of antibody: SC16.8 VL CDR3
LQFYEFPFTFGAGTKLELK

SEQ ID NO: 7 - protein, 121 aa, synthetic construct
  REGION 1..121: note = Description of sequence: component of antibody: SC16.8 full VH
QAQLQQSGAELVRPGTSVKVSCKASGYAFTNYLIEWVKQRPGQGLEWIGVINPGTGGTNYNENFKGKATLTADKSSSTAYMQLSSLTSDDSAVYFCARSPYDYHEGAMDYWGQGTSVTVSS

SEQ ID NO: 8 - protein, 107 aa, synthetic construct
  REGION 1..107: note = Description of sequence: component of antibody: SC16.8 full VL
EIQMTQSPSSMSASLGDRITITCQATQDIVKNLNWYQQKPGKPPSFLIYYAIELAEGVPSRFSGSGSGSDYSLTISNLESEDFADYYCLQFYEFPFTFGAGTKLELK

SEQ ID NO: 9 - DNA, 363 bp, synthetic construct
  misc_feature 1..363: note = Description of sequence: coding sequence of antibody: SC16.8 full VH DNA
  source 1..363: mol_type = other DNA
caggcccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaactggtggtactaactacaatgagaacttcaagggcaaggcaactctgactgcagacaaatcctccagtactgcctacatgcagctcagcagcctgacatctgatgactctgcggtctatttctgtgcaagatccccctatgattaccacgagggtgctatggactactggggtcaaggaacctcagtcaccgtctcctca

SEQ ID NO: 10 - DNA, 322 bp, synthetic construct
  misc_feature 1..322: note = Description of sequence: coding sequence of antibody: SC16.8 full VL DNA
  source 1..322: mol_type = other DNA
gaaatccagatgacccagtctccatcctctatgtctgcatctctgggagacagaataaccatcacttgccaggcaactcaagacattgttaagaatttaaactggtatcagcagaaaccagggaaacccccttcattcctgatctattatgcaattgaactggcagaaggggtcccatcaaggttcagtggcagtgggtctgggtcagactattctctgacaatcagcaacctggagtctgaagattttgcagactattactgtctacagttttatgagtttccgttcacgttcggtgctgggaccaagctggagctgaaac

SEQ ID NO: 11 - protein, 10 aa, synthetic construct
  REGION 1..10: note = Description of sequence: component of antibody: SC16.13/SC16.25 VH CDR1
GFSLSTSGMG

SEQ ID NO: 12 - protein, 7 aa, synthetic construct
  REGION 1..7: note = Description of sequence: component of antibody: SC16.13 VH CDR2
IWWDDVK

SEQ ID NO: 13 - protein, 27 aa, synthetic construct
  REGION 1..27: note = Description of sequence: component of antibody: SC16.13 VH CDR3
ARIVSFDNDVVSAMDYWGQGTSVTVSS

SEQ ID NO: 14 - protein, 8 aa, synthetic construct
  REGION 1..8: note = Description of sequence: component of antibody: SC16.13/SC16.25 VL CDR1
SASSSVSY

SEQ ID NO: 15 - protein, 7 aa, synthetic construct
  REGION 1..7: note = Description of sequence: component of antibody: SC16.13 VL CDR2
LTSNLAS

SEQ ID NO: 16 - protein, 19 aa, synthetic construct
  REGION 1..19: note = Description of sequence: component of antibody: SC16.13 VL CDR3
QQWRSNPFTFGSGTKLEIK

SEQ ID NO: 17 - protein, 124 aa, synthetic construct
  REGION 1..124: note = Description of sequence: component of antibody: SC16.13 full VH
QVTLKESGPGILQPSQTLSLTCSFSGFSLSTSGMGVGWIRQPSGKGLEWLAHIWWDDVKRYNPALKSRLTISKDTSSSQVFLKIASVDTADTATYYCARIVSFDNDVVSAMDYWGQGTSVTVSS

SEQ ID NO: 18 - protein, 106 aa, synthetic construct
  REGION 1..106: note = Description of sequence: component of antibody: SC16.13 full VL
QIVLTQSPALVSASPGEKVTMTCSASSSVSYMYWYQQKPRSSPKPWIYLTSNLASGVPARFSGSGSGTSYSLTISSMEAEDAATYYCQQWRSNPFTFGSGTKLEIK

SEQ ID NO: 19 - DNA, 371 bp, synthetic construct
  misc_feature 1..371: note = Description of sequence: coding sequence of antibody: SC16.13 full VH DNA
  source 1..371: mol_type = other DNA
caggttactctgaaagagtctggccctgggatattgcagccctcccagaccctcagtctgacttgttctttctctgggttttcactgagcacttctggtatgggtgtaggctggattcggcagccatcagggaagggtctggagtggctggcacacatttggtgggatgatgtcaagcgctataacccagccctgaagagccgactgactatctccaaggatacctccagcagccaggtattcctcaagatcgccagtgtggacactgcagatactgccacatactactgtgctcgcatagtttcctttgataacgacgttgtctctgctatggactactggggtcaaggaacctcagtcaccgtctcctc

SEQ ID NO: 20 - DNA, 319 bp, synthetic construct
  misc_feature 1..319: note = Description of sequence: coding sequence of antibody: SC16.13 full VL DNA
  source 1..319: mol_type = other DNA
caaattgttctcacccagtctccagcactcgtgtctgcatctccaggggagaaggtcaccatgacctgcagtgccagctcaagtgtaagttacatgtactggtaccagcagaaaccaagatcctcccccaaaccctggatttatctcacatccaacctggcttctggagtccctgctcgcttcagtggcagtgggtctgggacctcttactctctcacaatcagcagcatggaggctgaagatgctgccacttattactgccagcagtggcgtagtaacccattcacgttcggctcggggacaaagttggaaataaaac

SEQ ID NO: 21 - protein, 9 aa, synthetic construct
  REGION 1..9: note = Description of sequence: component of antibody: SC16.15 VH CDR1
SGYTFTRYW

SEQ ID NO: 22 - protein, 8 aa, synthetic construct
  REGION 1..8: note = Description of sequence: component of antibody: SC16.15 VH CDR2
INPTTVYT

SEQ ID NO: 23 - protein, 21 aa, synthetic construct
  REGION 1..21: note = Description of sequence: component of antibody: SC16.15 VH CDR3
ARGGSNFFDYWGQGTTLTVSS

SEQ ID NO: 24 - protein, 9 aa, synthetic construct
  REGION 1..9: note = Description of sequence: component of antibody: SC16.15 VL CDR1
RASENIYYN

SEQ ID NO: 25 - protein, 7 aa, synthetic construct
  REGION 1..7: note = Description of sequence: component of antibody: SC16.15 VL CDR2
TANSLED

SEQ ID NO: 26 - protein, 19 aa, synthetic construct
  REGION 1..19: note = Description of sequence: component of antibody: SC16.15 VL CDR3
KQAYDVPPTFGGGTKLEIK

SEQ ID NO: 27 - protein, 117 aa, synthetic construct
  REGION 1..117: note = Description of sequence: component of antibody: SC16.15 full VH
QVQLQQSGAELAKPGASVKMSCKASGYTFTRYWIHWIKQRPGQGLEWIGYINPTTVYTEFNQNFKDKATLTADKSSTTASMQLSSLTSEDSAVYYCARGGSNFFDYWGQGTTLTVSS

SEQ ID NO: 28 - protein, 107 aa, synthetic construct
  REGION 1..107: note = Description of sequence: component of antibody: SC16.15 full VL
DIQMTQSPASLAASVGETVAITCRASENIYYNLAWYQQKQGKSPQLLIYTANSLEDGVPSRFSGSGSGTQYSLKINSMQPEDSATYFCKQAYDVPPTFGGGTKLEIK

SEQ ID NO: 29 - DNA, 351 bp, synthetic construct
  misc_feature 1..351: note = Description of sequence: coding sequence of antibody: SC16.15 full VH DNA
  source 1..351: mol_type = other DNA
caggtccagcttcagcagtctggggctgaactggcaaaacctggggcctcagtgaagatgtcctgtaaggcttctggctacacctttactcgctactggatacactggataaaacagaggcctggacagggtctggaatggattggatacattaatcctacaactgtttatactgagttcaatcagaacttcaaggacaaggccactttgactgcagacaaatcctccaccacagcctccatgcaactgagcagcctgacatctgaggactctgcagtctattactgtgcaagaggcggtagtaacttctttgactactggggccaaggcaccactctcacagtctcctca

SEQ ID NO: 30 - DNA, 322 bp, synthetic construct
  misc_feature 1..322: note = Description of sequence: coding sequence of antibody: SC16.15 full VL DNA
  source 1..322: mol_type = other DNA
gacatccagatgactcagtctccagcctccctggctgcatctgtgggagaaactgtcgccatcacatgtcgagcaagtgagaacatttactacaatttagcatggtatcagcagaaacaagggaaatctcctcagctcctgatctatactgcaaacagtttggaagatggtgtcccatcgaggttcagtggcagtggatctgggacacagtattctttgaagatcaacagcatgcagcctgaagattccgcaacttatttctgtaaacaggcttatgacgttcctccgacgttcggtggaggcaccaagctggaaatcaaac

SEQ ID NO: 31 - protein, 7 aa, synthetic construct
  REGION 1..7: note = Description of sequence: component of antibody: SC16.25 VH CDR2
IWWDDNK

SEQ ID NO: 32 - protein, 27 aa, synthetic construct
  REGION 1..27: note = Description of sequence: component of antibody: SC16.25 VH CDR3
ARRVNYYYDPYYAMDYWGQGTSVTVSS

SEQ ID NO: 33 - protein, 7 aa, synthetic construct
  REGION 1..7: note = Description of sequence: component of antibody: SC16.25 VL CDR2
DSSKLAS

SEQ ID NO: 34 - protein, 19 aa, synthetic construct
  REGION 1..19: note = Description of sequence: component of antibody: SC16.25 VL CDR3
QQWSSNPLTFGAGTKLELK

SEQ ID NO: 35 - protein, 124 aa, synthetic construct
  REGION 1..124: note = Description of sequence: component of antibody: SC16.25 full VH
QVTLKESGPGILQPSQTLSLTCSFSGFSLSTSGMGVGWIRQPSGEGLEWLTDIWWDDNKYYNPSLKSRLTISKDTSSNQVFLNITSVDTADTATYYCARRVNYYYDPYYAMDYWGQGTSVTVSS

SEQ ID NO: 36 - protein, 106 aa, synthetic construct
  REGION 1..106: note = Description of sequence: component of antibody: SC16.25 full VL
QIVLTQSPAIMSASPGEKVTMTCSASSSVSYMHWYQQKSGTSPKRWIYDSSKLASGVPARFSGSGSGTSYSLTISSMEAEDAATYYCQQWSSNPLTFGAGTKLELK

SEQ ID NO: 37 - DNA, 372 bp, synthetic construct
  misc_feature 1..372: note = Description of sequence: coding sequence of antibody: SC16.25 full VH DNA
  source 1..372: mol_type = other DNA
caggttactctgaaagagtctggccctgggatattgcagccctcccagaccctcagtctgacttgttctttctctgggttttcactgagcacttctggtatgggtgtaggctggattcgtcagccttcaggagagggtctagagtggctgacagacatttggtgggatgacaataagtactataacccatccctgaagagccggctcacaatctccaaggatacctccagcaaccaggtattcctcaatatcaccagtgtggacactgcagatactgccacttactactgtgctcgaagagttaactattattacgacccgtactatgctatggactactggggtcaaggaacctcagtcaccgtctcctca

SEQ ID NO: 38 - DNA, 319 bp, synthetic construct
  misc_feature 1..319: note = Description of sequence: coding sequence of antibody: SC16.25 full VL DNA
  source 1..319: mol_type = other DNA
caaattgttctcacccagtctccagcaatcatgtctgcatctccaggggagaaggtcaccatgacctgcagtgccagctcaagtgtaagttacatgcactggtaccagcagaagtcaggcacctcccccaaaagatggatttatgactcatccaaactggcttctggagtccctgctcgcttcagtggcagtgggtctgggacctcttactctctcacaatcagcagcatggaggctgaagatgctgccacttattactgccagcagtggagtagtaacccgctcacgttcggtgctgggaccaagctggagctgaaac

SEQ ID NO: 39 - protein, 8 aa, synthetic construct
  REGION 1..8: note = Description of sequence: component of antibody: SC16.67 VH CDR1
AFTFTTYA

SEQ ID NO: 40 - protein, 10 aa, synthetic construct
  REGION 1..10: note = Description of sequence: component of antibody: SC16.67 VH CDR2
IRNKSNNYAT

SEQ ID NO: 41 - protein, 19 aa, synthetic construct
  REGION 1..19: note = Description of sequence: component of antibody: SC16.67 VH CDR3
VFYYDYVYWGQGTLVTVSA

SEQ ID NO: 42 - protein, 12 aa, synthetic construct
  REGION 1..12: note = Description of sequence: component of antibody: SC16.67 VL CDR1
RSSTGAVTTSNY

SEQ ID NO: 43 - protein, 7 aa, synthetic construct
  REGION 1..7: note = Description of sequence: component of antibody: SC16.67 VL CDR2
GTNNRAP

SEQ ID NO: 44 - protein, 19 aa, synthetic construct
  REGION 1..19: note = Description of sequence: component of antibody: SC16.67 VL CDR3
GLWYSNHLVFGGGTKLTVL

SEQ ID NO: 45 - protein, 117 aa, synthetic construct
  REGION 1..117: note = Description of sequence: component of antibody: SC16.67 full VH
EVQLVETGGGLVQPKGSLKLSCAVSAFTFTTYAMNWVRQAPGKGLEWVARIRNKSNNYATYYADSVKDRFTISRDDSQSMLYLQMNNLKIEDTAMYYCVFYYDYVYWGQGTLVTVSA

SEQ ID NO: 46 - protein, 109 aa, synthetic construct
  REGION 1..109: note = Description of sequence: component of antibody: SC16.67 full VL
QAVVTQESALTTSPGETVTLTCRSSTGAVTTSNYANWIQEKPDHLFTGLIGGTNNRAPGVPARFSGSLIGDKAALTITGAQTEDEAIYFCGLWYSNHLVFGGGTKLTVL

SEQ ID NO: 47 - DNA, 351 bp, synthetic construct
  misc_feature 1..351: note = Description of sequence: coding sequence of antibody: SC16.67 full VH DNA
  source 1..351: mol_type = other DNA
gaggtgcagcttgttgagactggtggaggattggtgcagcctaaagggtcattgaaactctcatgtgcagtctctgcattcaccttcactacctacgccatgaactgggtccgccaggctccaggaaagggtttggagtgggttgctcgcataagaaataaaagtaataattatgcaacatattatgccgattcagtgaaagacaggttcaccatctccagagatgattcacaaagcatgctctatctgcaaatgaacaacttgaaaattgaggacacagccatgtattactgtgtgttctactatgattacgtctactggggccaagggactctggtcactgtctctgca

SEQ ID NO: 48 - DNA, 328 bp, synthetic construct
  misc_feature 1..328: note = Description of sequence: coding sequence of antibody: SC16.67 full VL DNA
  source 1..328: mol_type = other DNA
caggctgttgtgactcaggaatctgcactcaccacatcacctggtgaaacagtcacactcacttgtcgctcaagtactggggctgttacaactagtaactatgccaactggatccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtggtctatggtacagcaaccatttggtgttcggtggaggaaccaaactgactgtcctag

SEQ ID NO: 49 - protein, 8 aa, synthetic construct
  REGION 1..8: note = Description of sequence: component of antibody: SC16.118 VH CDR1
GYSFTGYY

SEQ ID NO: 50 - protein, 8 aa, synthetic construct
  REGION 1..8: note = Description of sequence: component of antibody: SC16.118 VH CDR2
VNPNNGGT

SEQ ID NO: 51 - protein, 21 aa, synthetic construct
  REGION 1..21: note = Description of sequence: component of antibody: SC16.118 VH CDR3
ARGSYDYAEGWGQGTLVTVSA

SEQ ID NO: 52 - protein, 13 aa, synthetic construct
  REGION 1..13: note = Description of sequence: component of antibody: SC16.118 VL CDR1
KASQSVDYDGDSY

SEQ ID NO: 53 - protein, 7 aa, synthetic construct
  REGION 1..7: note = Description of sequence: component of antibody: SC16.118 VL CDR2
AASNLES

SEQ ID NO: 54 - protein, 20 aa, synthetic construct
  REGION 1..20: note = Description of sequence: component of antibody: SC16.118 VL CDR3
QQSNEDPYTFGGGTKLEIKR

SEQ ID NO: 55 - protein, 117 aa, synthetic construct
  REGION 1..117: note = Description of sequence: component of antibody: SC16.118 full VH
EVQLQQSGPDLVKPGASVKISCKASGYSFTGYYMHWVKQSHGKSLEWIGRVNPNNGGTSYNQKFKGKAILTADKSSSTAYMELRSLTSEDSAVYYCARGSYDYAEGWGQGTLVTVSA

SEQ ID NO: 56 - protein, 112 aa, synthetic construct
  REGION 1..112: note = Description of sequence: component of antibody: SC16.118 full VL
DIVLTQSPASLAVSLGQRATISCKASQSVDYDGDSYLTWYQQKPGQPPKLLIYAASNLESGIPARFSGSGSGTDFTLNIHPVEEEDAATYYCQQSNEDPYTFGGGTKLEIKR

SEQ ID NO: 57 - DNA, 351 bp, synthetic construct
  misc_feature 1..351: note = Description of sequence: coding sequence of antibody: SC16.118 full VH DNA
  source 1..351: mol_type = other DNA
gaggtccagctgcagcagtctggacctgacctggtgaagcctggggcttcagtgaagatatcctgcaaggcttctggttactcattcactggctactacatgcactgggtgaagcagagccatggaaagagccttgagtggattggacgtgttaatcctaacaatggtggtactagctacaaccagaagttcaagggcaaggccatattaactgcagacaagtcatccagcacagcctacatggagctccgcagcctgacatctgaggactctgcggtctattactgtgcaagagggagttatgattacgccgagggctggggccaagggactctggtcactgtctctgca

SEQ ID NO: 58 - DNA, 335 bp, synthetic construct
  misc_feature 1..335: note = Description of sequence: coding sequence of antibody: SC16.118 full VL DNA
  source 1..335: mol_type = other DNA
gacattgtgctgacccaatctccagcttctttggctgtgtctctagggcagagggccaccatctcctgcaaggccagccaaagtgttgattatgatggtgatagttatttgacctggtaccaacagaaaccaggacagccacccaaactcctcatctatgctgcatccaatctagaatctgggatcccagccaggtttagtggcagtgggtctgggacagacttcaccctcaacatccatcctgtggaggaggaggacgctgcaacctattactgtcagcaaagtaatgaggatccgtacacgttcggaggggggaccaagctggaaataaaacg

SEQ ID NO: 59 - protein, 9 aa, synthetic construct
  REGION 1..9: note = Description of sequence: component of antibody: SC16.125 VH CDR1
GYSITSGYS

SEQ ID NO: 60 - protein, 7 aa, synthetic construct
  REGION 1..7: note = Description of sequence: component of antibody: SC16.125 VH CDR2
IHYSGST

SEQ ID NO: 61 - protein, 21 aa, synthetic construct
  REGION 1..21: note = Description of sequence: component of antibody: SC16.125 VH CDR3
ALEGNYDGFAYWGQGTLVTVS

SEQ ID NO: 62 - protein, 9 aa, synthetic construct
  REGION 1..9: note = Description of sequence: component of antibody: SC16.125/SC16.125 VL CDR1
HASQNINVW

SEQ ID NO: 63 - protein, 7 aa, synthetic construct
  REGION 1..7: note = Description of sequence: component of antibody: SC16.125 VL CDR2
KASILHT

SEQ ID NO: 64 - protein, 19 aa, synthetic construct
  REGION 1..19: note = Description of sequence: component of antibody: SC16.125 VL CDR3
QQGQSYPYTFGGGTKLEIK

SEQ ID NO: 65 - protein, 118 aa, synthetic construct
  REGION 1..118: note = Description of sequence: component of antibody: SC16.125 full VH
SDVQLQESGPDLVKPSQSLSLTCTVTGYSITSGYSWHWIRQFPGNKLEWMGYIHYSGSTNYNPSLKSRISITRDTSKNQFFLQFKSVTTEDSATYYCALEGNYDGFAYWGQGTLVTVS

SEQ ID NO: 66 - protein, 107 aa, synthetic construct
  REGION 1..107: note = Description of sequence: component of antibody: SC16.125 full VL
DIQMNQSPSSLSASLGDTITITCHASQNINVWLSWYQQKPGNIPKLLIYKASILHTGVPSRFSGSGSGTGFTLTISSLQPEDIATYSCQQGQSYPYTFGGGTKLEIK

SEQ ID NO: 67 - DNA, 355 bp, synthetic construct
  misc_feature 1..355: note = Description of sequence: coding sequence of antibody: SC16.125 full VH DNA
  source 1..355: mol_type = other DNA
tctgatgtgcagcttcaggagtcaggacctgacctggtgaaaccttctcagtcactttcactcacctgcactgtcactggctactccatcaccagtggttatagctggcactggatccggcagtttccaggaaacaaactggaatggatgggctacatacactacagtggtagcactaactacaacccatctctcaaaagtcgaatctctatcactcgagacacatccaagaaccagttcttcctgcagttcaaatctgtgactactgaagactcagccacatattactgtgccctagaggggaattacgacgggtttgcttactggggccaagggactctggtcactgtctctg

SEQ ID NO: 68 - DNA, 321 bp, synthetic construct
  misc_feature 1..321: note = Description of sequence: coding sequence of antibody: SC16.125 full VL DNA
  source 1..321: mol_type = other DNA
gacatccagatgaaccagtctccatccagtctgtctgcatcccttggagacacaattaccatcacttgccatgccagtcagaacattaatgtttggttaagctggtaccagcagaaaccaggaaatattcctaaactattgatctataaggcttccatcttacacacaggcgtcccatcaaggtttagtggcagtggatctggaacaggtttcacattaaccatcagcagcctgcagcctgaagacattgccacttactcctgtcaacagggtcaaagttatccgtacacgttcggaggggggaccaagctggaaataaaa

SEQ ID NO: 69 - protein, 8 aa, synthetic construct
  REGION 1..8: note = Description of sequence: component of antibody: SC16.126 VH CDR1
GSSLTNYG

SEQ ID NO: 70 - protein, 7 aa, synthetic construct
  REGION 1..7: note = Description of sequence: component of antibody: SC16.126 VH CDR2
IWAGGST

SEQ ID NO: 71 - protein, 21 aa, synthetic construct
  REGION 1..21: note = Description of sequence: component of antibody: SC16.126 VH CDR3
ARDWEGWFAYWGQGTLVTVSA

SEQ ID NO: 72 - protein, 7 aa, synthetic construct
  REGION 1..7: note = Description of sequence: component of antibody: SC16.126 VL CDR2
KASNLHT

SEQ ID NO: 73 - protein, 19 aa, synthetic construct
  REGION 1..19: note = Description of sequence: component of antibody: SC16.126 VL CDR3
QQGQSYPFTFGSGTKLEIK

SEQ ID NO: 74 - protein, 116 aa, synthetic construct
  REGION 1..116: note = Description of sequence: component of antibody: SC16.126 full VH
QVQMKESGPGLVAPSQSLSITCTVSGSSLTNYGVHWVRQPPGKGLEWLGVIWAGGSTNYNSALMSRLSISKDNSKSQVFLKMNSLQTDDTAMYYCARDWEGWFAYWGQGTLVTVSA

SEQ ID NO: 75 - protein, 107 aa, synthetic construct
  REGION 1..107: note = Description of sequence: component of antibody: SC16.126 full VL
DIQMNQSPSSLSASLGDTITITCHASQNINVWLSWYQQKPGNIPKLLIYKASNLHTGVPSRFSGSGSGTGFTLTISSLQPEDIATYYCQQGQSYPFTFGSGTKLEIK

SEQ ID NO: 76 - DNA, 348 bp, synthetic construct
  misc_feature 1..348: note = Description of sequence: coding sequence of antibody: SC16.126 full VH DNA
  source 1..348: mol_type = other DNA
caggtgcagatgaaggagtcaggacctggcctggtggcgccctcacagagcctgtccatcacttgcactgtctctgggtcttcattaaccaactatggtgtacactgggttcgccagcctccaggaaagggtctagagtggctgggagtaatatgggctggtggaagcacaaattataattcggctctcatgtccagactgagtatcagcaaagacaactccaagagccaagttttcttaaaaatgaacagtctgcaaactgatgacacagccatgtactactgtgccagagactgggagggctggtttgcttactggggccaagggactctggtcactgtctctgca

SEQ ID NO: 77 - DNA, 322 bp, synthetic construct
  misc_feature 1..322: note = Description of sequence: coding sequence of antibody: SC16.126 full VL DNA
  source 1..322: mol_type = other DNA
gacatccagatgaaccagtctccatccagtctgtctgcatcccttggagacacaattaccatcacttgccatgccagtcagaacataaatgtttggttaagctggtaccagcagaaaccaggaaatattcctaaactattgatctataaggcttccaacttgcacacaggcgtcccatcaaggtttagtggcagtggatctggaacaggtttcacattaaccatcagcagcctgcagcctgaagacattgccacttactactgtcaacagggtcaaagttatccattcacgttcggctcggggacaaagttggaaataaaac

SEQ ID NO: 78 - protein, 19 aa, synthetic construct
  REGION 1..19: note = Description of sequence: Linker 1
GGGGSGGGGSGGGSGGGGS

SEQ ID NO: 79 - protein, 15 aa, synthetic construct
  REGION 1..15: note = Description of sequence: Linker 2
GGGGSGGGGSGGGGS

SEQ ID NO: 80 - protein, 24 aa, synthetic construct
  REGION 1..24: note = Description of sequence: Linker 3
GGGGSGGGGSGGGGSGGGSGGGGS

SEQ ID NO: 81 - protein, 29 aa, synthetic construct
  REGION 1..29: note = Description of sequence: Linker 4
GGGGSGGGGSGGGGSGGGGSGGGSGGGGS

SEQ ID NO: 82 - protein, 5 aa, synthetic construct
  REGION 1..5: note = Description of sequence: Linker 5
GGGGS

SEQ ID NO: 83 - protein, 10 aa, synthetic construct
  REGION 1..10: note = Description of sequence: Linker 6
GGGGSGGGGS

SEQ ID NO: 84 - protein, 618 aa, synthetic construct
  REGION 1..618: note = Description of sequence: Human DLL3
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 85 - protein, 235 aa, synthetic construct
  REGION 1..235: note = Description of sequence: human CD8
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 86 - protein, 247 aa, synthetic construct
  REGION 1..247: note = Description of sequence: Mouse CD8
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 87 - protein, 220 aa, synthetic construct
  REGION 1..220: note = Description of sequence: Human CD28
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 88 - protein, 218 aa, synthetic construct
  REGION 1..218: note = Description of sequence: Mouse CD28
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 89 - protein, 164 aa, synthetic construct
  REGION 1..164: note = Description of sequence: Human full-length CD3-Zeta polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 90 - protein, 112 aa, synthetic construct
  REGION 1..112: note = Description of sequence: Intracellular CD3-Zeta polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - DNA, 336 bp, synthetic construct
  misc_feature 1..336: note = Description of sequence: Nucleotide of SEQ ID NO: 90
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 92 - protein, 4 aa, synthetic construct
  REGION 1..4: note = Description of sequence: YMNM motif
YMNM

SEQ ID NO: 93-94 - skipped (2 entries)

SEQ ID NO: 95 - protein, 4 aa, synthetic construct
  REGION 1..4: note = Description of sequence: YENV motif
YENV

SEQ ID NO: 96 - protein, 4 aa, synthetic construct
  REGION 1..4: note = Description of sequence: YSNV motif
YSNV

SEQ ID NO: 97 - protein, 4 aa, synthetic construct
  REGION 1..4: note = Description of sequence: YKNL motif
YKNL

SEQ ID NO: 98 - protein, 4 aa, synthetic construct
  REGION 1..4: note = Description of sequence: YENQ motif
YENQ

SEQ ID NO: 99 - protein, 4 aa, synthetic construct
  REGION 1..4: note = Description of sequence: YKNI motif
YKNI

SEQ ID NO: 100 - protein, 4 aa, synthetic construct
  REGION 1..4: note = Description of sequence: YINQ motif
YINQ

SEQ ID NO: 101 - protein, 4 aa, synthetic construct
  REGION 1..4: note = Description of sequence: YHNK motif
YHNK

SEQ ID NO: 102 - protein, 4 aa, synthetic construct
  REGION 1..4: note = Description of sequence: YVNQ motif
YVNQ

SEQ ID NO: 103 - protein, 4 aa, synthetic construct
  REGION 1..4: note = Description of sequence: YLNP motif
YLNP

SEQ ID NO: 104 - protein, 4 aa, synthetic construct
  REGION 1..4: note = Description of sequence: YLNT motif
YLNT

SEQ ID NO: 105 - protein, 4 aa, synthetic construct
  REGION 1..4: note = Description of sequence: YDND motif
YDND

SEQ ID NO: 106 - protein, 4 aa, synthetic construct
  REGION 1..4: note = Description of sequence: YENI motif
YENI

SEQ ID NO: 107 - protein, 4 aa, synthetic construct
  REGION 1..4: note = Description of sequence: YENL motif
YENL

SEQ ID NO: 108 - protein, 4 aa, synthetic construct
  REGION 1..4: note = Description of sequence: YKNQ motif
YKNQ

SEQ ID NO: 109 - protein, 4 aa, synthetic construct
  REGION 1..4: note = Description of sequence: YKNV motif
YKNV

SEQ ID NO: 110 - protein, 4 aa, synthetic construct
  REGION 1..4: note = Description of sequence: YANG motif
YANG

SEQ ID NO: 111 - protein, 4 aa, synthetic construct
  REGION 1..4: note = Description of sequence: YMDM motif
YMDM

SEQ ID NO: 112 - protein, 4 aa, synthetic construct
  REGION 1..4: note = Description of sequence: YMPM motif
YMPM

SEQ ID NO: 113 - protein, 4 aa, synthetic construct
  REGION 1..4: note = Description of sequence: YMRM motif
YMRM

SEQ ID NO: 114 - protein, 4 aa, synthetic construct
  REGION 1..4: note = Description of sequence: YMSM motif
YMSM

SEQ ID NO: 115 - skipped

SEQ ID NO: 116 - protein, 4 aa, synthetic construct
  REGION 1..4: note = Description of sequence: YTHM motif
YTHM

SEQ ID NO: 117 - protein, 4 aa, synthetic construct
  REGION 1..4: note = Description of sequence: YVLM motif
YVLM

SEQ ID NO: 118 - protein, 4 aa, synthetic construct
  REGION 1..4: note = Description of sequence: YIAM motif
YIAM

SEQ ID NO: 119 - protein, 4 aa, synthetic construct
  REGION 1..4: note = Description of sequence: YVEM motif
YVEM

SEQ ID NO: 120 - protein, 4 aa, synthetic construct
  REGION 1..4: note = Description of sequence: YVKM motif
YVKM

SEQ ID NO: 121 - protein, 4 aa, synthetic construct
  REGION 1..4: note = Description of sequence: YVPM motif
YVPM

SEQ ID NO: 122 - skipped

SEQ ID NO: 123 - protein, 4 aa, synthetic construct
  REGION 1..4: note = Description of sequence: YMAP motif
YMAP

SEQ ID NO: 124-125 - skipped (2 entries)

SEQ ID NO: 126 - protein, 4 aa, synthetic construct
  REGION 1..4: note = Description of sequence: YMNV motif
YMNV

SEQ ID NO: 127 - protein, 4 aa, synthetic construct
  REGION 1..4: note = Description of sequence: YENM motif
YENM

SEQ ID NO: 128 - protein, 4 aa, synthetic construct
  REGION 1..4: note = Description of sequence: YMNQ motif
YMNQ

SEQ ID NO: 129 - protein, 4 aa, synthetic construct
  REGION 1..4: note = Description of sequence: YMNL motif
YMNL

SEQ ID NO: 130 - protein, 4 aa, synthetic construct
  REGION 1..4: note = Description of sequence: YSNM motif
YSNM

SEQ ID NO: 131 - skipped

SEQ ID NO: 132 - protein, 4 aa, synthetic construct
  REGION 1..4: note = Description of sequence: YGGG peptide
YGGG

SEQ ID NO: 133 - protein, 4 aa, synthetic construct
  REGION 1..4: note = Description of sequence: YAAA peptide
YAAA

SEQ ID NO: 134 - protein, 4 aa, synthetic construct
  REGION 1..4: note = Description of sequence: YFFF peptide
YFFF

SEQ ID NO: 135 - protein, 4 aa, synthetic construct
  REGION 1..4: note = Description of sequence: YETV peptide
YETV

SEQ ID NO: 136 - protein, 4 aa, synthetic construct
  REGION 1..4: note = Description of sequence: YQQQ peptide
YQQQ

SEQ ID NO: 137 - protein, 4 aa, synthetic construct
  REGION 1..4: note = Description of sequence: YHAE peptide
YHAE

SEQ ID NO: 138 - protein, 4 aa, synthetic construct
  REGION 1..4: note = Description of sequence: YLDL peptide
YLDL

SEQ ID NO: 139 - protein, 4 aa, synthetic construct
  REGION 1..4: note = Description of sequence: YLIP peptide
YLIP

SEQ ID NO: 140 - protein, 4 aa, synthetic construct
  REGION 1..4: note = Description of sequence: YLRV peptide
YLRV

SEQ ID NO: 141 - protein, 4 aa, synthetic construct
  REGION 1..4: note = Description of sequence: YTAV peptide
YTAV

SEQ ID NO: 142 - protein, 4 aa, synthetic construct
  REGION 1..4: note = Description of sequence: YVHV peptide
YVHV

SEQ ID NO: 143 - protein, 41 aa, synthetic construct
  REGION 1..41: note = Description of sequence: CD28 polypeptide 3
RSKRSRLLHSDYENVTPRRPGPTRKHYQPYAPPRDFAAYRS

SEQ ID NO: 144 - protein, 41 aa, synthetic construct
  REGION 1..41: note = Description of sequence: CD28 polypeptide 4
RSKRSRLLHSDYKNITPRRPGPTRKHYQPYAPPRDFAAYRS

SEQ ID NO: 145 - protein, 41 aa, synthetic construct
  REGION 1..41: note = Description of sequence: CD28 polypeptide 5
RSKRSRLLHSDYMDMTPRRPGPTRKHYQPYAPPRDFAAYRS

SEQ ID NO: 146 - protein, 41 aa, synthetic construct
  REGION 1..41: note = Description of sequence: CD28 polypeptide 6
RSKRSRLLHSDYGGGTPRRPGPTRKHYQPYAPPRDFAAYRS

SEQ ID NO: 147 - protein, 41 aa, synthetic construct
  REGION 1..41: note = Description of sequence: CD28 polypeptide 7
RSKRSRLLHSDYSNVTPRRPGPTRKHYQPYAPPRDFAAYRS

SEQ ID NO: 148 - protein, 255 aa, synthetic construct
  REGION 1..255: note = Description of sequence: 4-1BB polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 149 - protein, 157 aa, synthetic construct
  REGION 1..157: note = Description of sequence: Human IL-18
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 150 - protein, 193 aa, synthetic construct
  REGION 1..193: note = Description of sequence: Secretable IL-18 polypeptide
MAAEPVEDNCINFVAMKFIDNTLYFIAEDDENLESDYFGKLESKLSVIRNINDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGARSIMFTVQNED

SEQ ID NO: 151 - RNA, 20 nt, synthetic construct
  misc_feature 1..20: note = Description of sequence: sgRNA
  source 1..20: mol_type = other RNA
cggctcgcaccgcgcgcggt